SEQUENCE LISTING (151 sequences)

SEQ ID NO: 1 - protein, 8 aa, Synthetic construct
GFNIKDDY

SEQ ID NO: 2 - protein, 8 aa, Synthetic construct
IDPADGHT

SEQ ID NO: 3 - protein, 12 aa, Synthetic construct
ARYGYGREVFDY

SEQ ID NO: 4 - protein, 10 aa, Synthetic construct
QSVDYEGDSY

SEQ ID NO: 5 - protein, 10 aa, Synthetic construct
QSVDYYGDSY

SEQ ID NO: 6 - protein, 10 aa, Synthetic construct
QSVDYDGDSY

SEQ ID NO: 7 - skipped

SEQ ID NO: 8 - protein, 9 aa, Synthetic construct
QQSNEDPWT

SEQ ID NO: 9 - protein, 119 aa, Synthetic construct
QVQLVQSGAEVKKPGASVKLSCTASGFNIKDDYIHWVKEAPGQGLEWIGRIDPADGHTKYAPKFQVKVTITADTSTSTAYLELSSLRSEDTAVYYCARYGYGREVFDYWGQGTTVTVSS

SEQ ID NO: 10 - protein, 119 aa, Synthetic construct
QVQLVQSGAEVKKPGASVKLSCTASGFNIKDDYIHWVKQAPGQGLEWIGRIDPADGHTKYAPKFQVKVTITADTSTSTAYLELSSLRSEDTAVYYCARYGYGREVFDYWGQGTTVTVSS

SEQ ID NO: 11 - protein, 119 aa, Synthetic construct
QVQLVQSGAEVKKPGASVKLSCTASGFNIKDDYIHWVKEAPGQGLEWIGRIDPADGHTKYAPKFQVKVTITAATSTSTAYLQLSSLRSEDTAVYYCARYGYGREVFDYWGQGTTVTVSS

SEQ ID NO: 12 - protein, 119 aa, Synthetic construct
QVQLVQSGAEVKKPGASVKLSCTASGFNIKDDYIHWVKQAPGQGLEWIGRIDPADGHTKYAPKFQVKVTITAATSTSTAYLQLSSLRSEDTAVYYCARYGYGREVFDYWGQGTTVTVSS

SEQ ID NO: 13 - protein, 111 aa, Synthetic construct
DIVMTQSPDSLAVSLGERATISCKSSQSVDYEGDSYLNWYQKKPGQPPKLLIYDASNLESGVPDRFSGSGSGTDFTLTISSLQAEDVAVYYCQQSNEDPWTFGQGTKVEIK

SEQ ID NO: 14 - protein, 111 aa, Synthetic construct
DIVMTQSPDSLAVSLGERATISCKSSQSVDYEGDSYLNWYQQKPGQPPKLLIYDASNLESGVPDRFSGSGSGTDFTLTISSLQAEDVAVYYCQQSNEDPWTFGQGTKVEIK

SEQ ID NO: 15 - protein, 111 aa, Synthetic construct
DIVMTQSPDSLAVSLGERATISCKSSQSVDYYGDSYLNWYQKKPGQPPKLLIYDASNLESGVPDRFSGSGSGTDFTLTISSLQAEDVAVYYCQQSNEDPWTFGQGTKVEIK

SEQ ID NO: 16 - protein, 111 aa, Synthetic construct
DIVMTQSPDSLAVSLGERATISCKSSQSVDYYGDSYLNWYQQKPGQPPKLLIYDASNLESGVPDRFSGSGSGTDFTLTISSLQAEDVAVYYCQQSNEDPWTFGQGTKVEIK

SEQ ID NO: 17 - protein, 111 aa, Synthetic construct
DIVMTQSPDSLAVSLGERATISCKSSQSVDYEGDSYLNWYQKKPGQPPKLLIYDASNLESGVPDRFSGSGSGTAFTLTISSLQAEDVAVYYCQQSNEDPWTFGQGTKVEIK

SEQ ID NO: 18 - protein, 111 aa, Synthetic construct
DIVMTQSPDSLAVSLGERATISCKSSQSVDYEGDSYLNWYQQKPGQPPKLLIYDASNLESGVPDRFSGSGSGTAFTLTISSLQAEDVAVYYCQQSNEDPWTFGQGTKVEIK

SEQ ID NO: 19 - protein, 111 aa, Synthetic construct
DIVMTQSPDSLAVSLGERATISCKSSQSVDYDGDSYLNWYQKKPGQPPKLLIYDASNLESGVPDRFSGSGSGTDFTLTISSLQAEDVAVYYCQQSNEDPWTFGQGTKVEIK

SEQ ID NO: 20 - protein, 111 aa, Synthetic construct
DIVLTQSPDSLAVSLGERATISCKASQSVDYDGDSYMNWYQKKPGQPPKILIYDASNLESGIPARFSGSGSGTDFTLTISSLEPEDFAIYYCQQSNEDPWTFGGGTKVEIK

SEQ ID NO: 21 - protein, 111 aa, Synthetic construct
DIVLTQSPDSLAVSLGERATISCKASQSVDYDGDSYMNWYQQKPGQPPKILIYDASNLESGIPARFSGSGSGTDFTLTISSLEPEDFAIYYCQQSNEDPWTFGGGTKVEIK

SEQ ID NO: 22 - protein, 8 aa, Synthetic construct
GYTFTRYY

SEQ ID NO: 23 - protein, 8 aa, Synthetic construct
IDPSVSET

SEQ ID NO: 24 - protein, 13 aa, Synthetic construct
SQIRLPYYYAMDS

SEQ ID NO: 25 - protein, 6 aa, Synthetic construct
QDIESF

SEQ ID NO: 26 - protein, 6 aa, Synthetic construct
QDISSF

SEQ ID NO: 27 - skipped

SEQ ID NO: 28 - protein, 9 aa, Synthetic construct
QQGNTLPRT

SEQ ID NO: 29 - protein, 120 aa, Synthetic construct
QVQLVQSGAEVKKPGASVKVSCKASGYTFTRYYLNWVRKAPGQGLEWIGMIDPSVSETHYAQKFQGRATLTVDKSTSTAYMELSSLRSEDTAVYYCSQIRLPYYYAMDSWGQGTTVTVSS

SEQ ID NO: 30 - protein, 120 aa, Synthetic construct
QVQLVQSGAEVKKPGASVKVSCKASGYTFTRYYLNWVRQAPGQGLEWIGMIDPSVSETHYAQKFQGRATLTVDKSTSTAYMELSSLRSEDTAVYYCSQIRLPYYYAMDSWGQGTTVTVSS

SEQ ID NO: 31 - protein, 107 aa, Synthetic construct
DIQMTQSPSSLSASVGDRVTITCRASQDIESFLNWYQEKPGKAPKLLIYYTSRLQSGVPSRFSGSGSGTDYTLTISSLQPEDFATYYCQQGNTLPRTFGGGTKVEIK

SEQ ID NO: 32 - protein, 107 aa, Synthetic construct
DIQMTQSPSSLSASVGDRVTITCRASQDIESFLNWYQQKPGKAPKLLIYYTSRLQSGVPSRFSGSGSGTDYTLTISSLQPEDFATYYCQQGNTLPRTFGGGTKVEIK

SEQ ID NO: 33 - protein, 107 aa, Synthetic construct
DIQMTQSPSSLSASVGDRVTITCRASQDISSFLNWYQEKPGKAPKLLIYYTSRLHSGVPSRFSGSGSGTDYTLTISSLQPEDFATYYCQQGNTLPRTFGGGTKVEIK

SEQ ID NO: 34 - protein, 107 aa, Synthetic construct
DIQMTQSPSSLSASVGDRVTITCRASQDISSFLNWYQQKPGKAPKLLIYYTSRLHSGVPSRFSGSGSGTDYTLTISSLQPEDFATYYCQQGNTLPRTFGGGTKVEIK

SEQ ID NO: 35 - protein, 107 aa, Synthetic construct
DIQMTQSPSSLSASVGDRVTITCRASQDISSFLNWYQEKPGKAPKLLIYYTSRLQSGVPSRFSGSGSGTDYTLTISSLQPEDFATYYCQQGNTLPRTFGGGTKVEIK

SEQ ID NO: 36 - protein, 107 aa, Synthetic construct
DIQMTQSPSSLSASVGDRVTITCRASQDISSFLNWYQQKPGKAPKLLIYYTSRLQSGVPSRFSGSGSGTDYTLTISSLQPEDFATYYCQQGNTLPRTFGGGTKVEIK

SEQ ID NO: 37 - protein, 330 aa, Synthetic construct [sequence omitted]

SEQ ID NO: 38 - protein, 329 aa, Synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLASSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 39 - protein, 329 aa, Synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLASSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 40 - protein, 327 aa, Synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 41 - protein, 329 aa, Synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLASSIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVLHEALHSHYTQKSLSLSPG

SEQ ID NO: 42 - protein, 329 aa, Synthetic construct
ASTKGPSVFPLAPSSKSTSGGTAALGCLVKDYFPEPVTVSWNSGALTSGVHTFPAVLQSSGLYSLSSVVEVPSSSLGTQTYICNVNHKPSNTKVDEKVEPDSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLASSIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVLHEALHSRFTQKSLSLSPG

SEQ ID NO: 43 - protein, 326 aa, Synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVLHEALHSHYTQKSLSLSLG

SEQ ID NO: 44 - protein, 326 aa, Synthetic construct
ASTKGPSVFPLAPCSRSTSESTAALGCLVKDYFPEPVTVSWNSGALTSGVHTFPAVLQSSGLYSLSSVVEVPSSSLGTKTYTCNVDHKPSNTKVDERVESDYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVLHEALHSRFTQKSLSLSLG

SEQ ID NO: 45 - protein, 107 aa, Synthetic construct [sequence omitted]

SEQ ID NO: 46 - protein, 107 aa, Synthetic construct
RTVAAPAVFIFPPSKKQLKSGTASVVCLLKNFYPREAKVQWKVDNALQSGNSQESVTEQDSKDSTYSLSSTLTLSKADYEKHKVYACEVTHQGLSSPVTKSFNRGEC

SEQ ID NO: 47 - protein, 445 aa, Synthetic construct
QVQLVQSGAEVKKPGASVKLSCTASGFNIKDDYIHWVKEAPGQGLEWIGRIDPADGHTKYAPKFQVKVTITADTSTSTAYLELSSLRSEDTAVYYCARYGYGREVFDYWGQGTTVTVSSASTKGPSVFPLAPCSRSTSESTAALGCLVKDYFPEPVTVSWNSGALTSGVHTFPAVLQSSGLYSLSSVVEVPSSSLGTKTYTCNVDHKPSNTKVDERVESDYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVLHEALHSRFTQKSLSLSLG

SEQ ID NO: 48 - protein, 445 aa, Synthetic construct
QVQLVQSGAEVKKPGASVKLSCTASGFNIKDDYIHWVKEAPGQGLEWIGRIDPADGHTKYAPKFQVKVTITAATSTSTAYLQLSSLRSEDTAVYYCARYGYGREVFDYWGQGTTVTVSSASTKGPSVFPLAPCSRSTSESTAALGCLVKDYFPEPVTVSWNSGALTSGVHTFPAVLQSSGLYSLSSVVEVPSSSLGTKTYTCNVDHKPSNTKVDERVESDYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVLHEALHSRFTQKSLSLSLG

SEQ ID NO: 49 - protein, 218 aa, Synthetic construct
DIVMTQSPDSLAVSLGERATISCKSSQSVDYEGDSYLNWYQKKPGQPPKLLIYDASNLESGVPDRFSGSGSGTDFTLTISSLQAEDVAVYYCQQSNEDPWTFGQGTKVEIKRTVAAPAVFIFPPSKKQLKSGTASVVCLLKNFYPREAKVQWKVDNALQSGNSQESVTEQDSKDSTYSLSSTLTLSKADYEKHKVYACEVTHQGLSSPVTKSFNRGEC

SEQ ID NO: 50 - protein, 218 aa, Synthetic construct
DIVMTQSPDSLAVSLGERATISCKSSQSVDYEGDSYLNWYQKKPGQPPKLLIYDASNLESGVPDRFSGSGSGTAFTLTISSLQAEDVAVYYCQQSNEDPWTFGQGTKVEIKRTVAAPAVFIFPPSKKQLKSGTASVVCLLKNFYPREAKVQWKVDNALQSGNSQESVTEQDSKDSTYSLSSTLTLSKADYEKHKVYACEVTHQGLSSPVTKSFNRGEC

SEQ ID NO: 51 - protein, 446 aa, Synthetic construct
QVQLVQSGAEVKKPGASVKVSCKASGYTFTRYYLNWVRKAPGQGLEWIGMIDPSVSETHYAQKFQGRATLTVDKSTSTAYMELSSLRSEDTAVYYCSQIRLPYYYAMDSWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVLHEALHSHYTQKSLSLSLG

SEQ ID NO: 52 - protein, 214 aa, Synthetic construct
DIQMTQSPSSLSASVGDRVTITCRASQDIESFLNWYQEKPGKAPKLLIYYTSRLQSGVPSRFSGSGSGTDYTLTISSLQPEDFATYYCQQGNTLPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 53 - protein, 688 aa, Homo sapiens
MWCIVLFSLLAWVYAEPTMYGEILSPNYPQAYPSEVEKSWDIEVPEGYGIHLYFTHLDIELSENCAYDSVQIISGDTEEGRLCGQRSSNNPHSPIVEEFQVPYNKLQVIFKSDFSNEERFTGFAAYYVATDINECTDFVDVPCSHFCNNFIGGYFCSCPPEYFLHDDMKNCGVNCSGDVFTALIGEIASPNYPKPYPENSRCEYQIRLEKGFQVVVTLRREDFDVEAADSAGNCLDSLVFVAGDRQFGPYCGHGFPGPLNIETKSNALDIIFQTDLTGQKKGWKLRYHGDPMPCPKEDTPNSVWEPAKAKYVFRDVVQITCLDGFEVVEGRVGATSFYSTCQSNGKWSNSKLKCQPVDCGIPESIENGKVEDPESTLFGSVIRYTCEEPYYYMENGGGGEYHCAGNGSWVNEVLGPELPKCVPVCGVPREPFEEKQRIIGGSDADIKNFPWQVFFDNPWAGGALINEYWVLTAAHVVEGNREPTMYVGSTSVQTSRLAKSKMLTPEHVFIHPGWKLLEVPEGRTNFDNDIALVRLKDPVKMGPTVSPICLPGTSSDYNLMDGDLGLISGWGRTEKRDRAVRLKAARLPVAPLRKCKEVKVEKPTADAEAYVFTPNMICAGGEKGMDSCKGDSGGAFAVQDPNDKTKFYAAGLVSWGPQCGTYGLYTRVKNYVDWIMKTMQENSTPRED

SEQ ID NO: 54 - protein, 760 aa, Homo sapiens
MMDQARSAFSNLFGGEPLSYTRFSLARQVDGDNSHVEMKLAVDEEENADNNTKANVTKPKRCSGSICYGTIAVIVFFLIGFMIGYLGYCKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 55 - protein, 5 aa, Synthetic construct
DDYIH

SEQ ID NO: 56 - protein, 17 aa, Synthetic construct
RIDPADGHTKYAPKFQV

SEQ ID NO: 57 - protein, 10 aa, Synthetic construct
YGYGREVFDY

SEQ ID NO: 58 - protein, 15 aa, Synthetic construct
KSSQSVDYEGDSYLN

SEQ ID NO: 59 - protein, 7 aa, Synthetic construct
DASNLES

SEQ ID NO: 60 - protein, 7 aa, Synthetic construct
GFNIKDD

SEQ ID NO: 61 - protein, 6 aa, Synthetic construct
DPADGH

SEQ ID NO: 62 - protein, 6 aa, Synthetic construct
KDDYIH

SEQ ID NO: 63 - protein, 13 aa, Synthetic construct
WIGRIDPADGHTK

SEQ ID NO: 64 - protein, 11 aa, Synthetic construct
ARYGYGREVFD

SEQ ID NO: 65 - protein, 11 aa, Synthetic construct
DYEGDSYLNWY

SEQ ID NO: 66 - protein, 10 aa, Synthetic construct
LLIYDASNLE

SEQ ID NO: 67 - protein, 8 aa, Synthetic construct
QQSNEDPW

SEQ ID NO: 68 - protein, 5 aa, Synthetic construct
RYYLN

SEQ ID NO: 69 - protein, 17 aa, Synthetic construct
MIDPSVSETHYAQKFQG

SEQ ID NO: 70 - protein, 11 aa, Synthetic construct
IRLPYYYAMDS

SEQ ID NO: 71 - protein, 11 aa, Synthetic construct
RASQDIESFLN

SEQ ID NO: 72 - protein, 7 aa, Synthetic construct
YTSRLQS

SEQ ID NO: 73 - protein, 7 aa, Synthetic construct
GYTFTRY

SEQ ID NO: 74 - protein, 6 aa, Synthetic construct
DPSVSE

SEQ ID NO: 75 - protein, 6 aa, Synthetic construct
TRYYLN

SEQ ID NO: 76 - protein, 13 aa, Synthetic construct
WIGMIDPSVSETH

SEQ ID NO: 77 - protein, 12 aa, Synthetic construct
SQIRLPYYYAMD

SEQ ID NO: 78 - protein, 7 aa, Synthetic construct
ESFLNWY

SEQ ID NO: 79 - protein, 10 aa, Synthetic construct
LLIYYTSRLQ

SEQ ID NO: 80 - protein, 8 aa, Synthetic construct
QQGNTLPR

SEQ ID NO: 81 - protein, 8 aa, Synthetic construct
GDTLTELS

SEQ ID NO: 82 - protein, 8 aa, Synthetic construct
FDPEEGET

SEQ ID NO: 83 - protein, 12 aa, Synthetic construct
VTEGLAGRPFDS

SEQ ID NO: 84 - protein, 6 aa, Synthetic construct
QSISSW

SEQ ID NO: 85 - skipped

SEQ ID NO: 86 - protein, 9 aa, Synthetic construct
QQYNSYSWT

SEQ ID NO: 87 - protein, 5 aa, Synthetic construct
ELSMH

SEQ ID NO: 88 - protein, 17 aa, Synthetic construct
TFDPEEGETIYAQKFQG

SEQ ID NO: 89 - protein, 10 aa, Synthetic construct
EGLAGRPFDS

SEQ ID NO: 90 - protein, 11 aa, Synthetic construct
RASQSISSWLA

SEQ ID NO: 91 - protein, 7 aa, Synthetic construct
KASSLES

SEQ ID NO: 92 - protein, 7 aa, Synthetic construct
GDTLTEL

SEQ ID NO: 93 - protein, 6 aa, Synthetic construct
DPEEGE

SEQ ID NO: 94 - protein, 6 aa, Synthetic construct
TELSMH

SEQ ID NO: 95 - protein, 13 aa, Synthetic construct
WMGTFDPEEGETI

SEQ ID NO: 96 - protein, 11 aa, Synthetic construct
VTEGLAGRPFD

SEQ ID NO: 97 - protein, 7 aa, Synthetic construct
SSWLAWY

SEQ ID NO: 98 - protein, 10 aa, Synthetic construct
LLIYKASSLE

SEQ ID NO: 99 - protein, 8 aa, Synthetic construct
QQYNSYSW

SEQ ID NO: 100 - protein, 119 aa, Synthetic construct
QVQLVQSGAEVKKPGASVKVSCKVSGDTLTELSMHWVRQAPGKGLEWMGTFDPEEGETIYAQKFQGRVTMTEDTSTDTAYMELSSLRSEDTAVYYCVTEGLAGRPFDSWGQGTLVTVSS

SEQ ID NO: 101 - protein, 107 aa, Synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYNSYSWTFGQGTKVEIK

SEQ ID NO: 102 - protein, 8 aa, Synthetic construct
GFTFSDYY

SEQ ID NO: 103 - protein, 8 aa, Synthetic construct
ISRSGSTK

SEQ ID NO: 104 - protein, 11 aa, Synthetic construct
ARDETDYALDY

SEQ ID NO: 105 - protein, 6 aa, Synthetic construct
QDISNY

SEQ ID NO: 106 - skipped

SEQ ID NO: 107 - protein, 9 aa, Synthetic construct
QQYEDLPLT

SEQ ID NO: 108 - protein, 5 aa, Synthetic construct
DYYMS

SEQ ID NO: 109 - protein, 17 aa, Synthetic construct
YISRSGSTKYYADSVKG

SEQ ID NO: 110 - protein, 9 aa, Synthetic construct
DETDYALDY

SEQ ID NO: 111 - protein, 11 aa, Synthetic construct
QASQDISNYLN

SEQ ID NO: 112 - protein, 7 aa, Synthetic construct
DASNLET

SEQ ID NO: 113 - protein, 7 aa, Synthetic construct
GFTFSDY

SEQ ID NO: 114 - protein, 6 aa, Synthetic construct
SRSGST

SEQ ID NO: 115 - protein, 6 aa, Synthetic construct
SDYYMS

SEQ ID NO: 116 - protein, 13 aa, Synthetic construct
WVSYISRSGSTKY

SEQ ID NO: 117 - protein, 10 aa, Synthetic construct
ARDETDYALD

SEQ ID NO: 118 - protein, 7 aa, Synthetic construct
SNYLNWY

SEQ ID NO: 119 - protein, 10 aa, Synthetic construct
LLIYDASNLE

SEQ ID NO: 120 - protein, 8 aa, Synthetic construct
QQYEDLPL

SEQ ID NO: 121 - protein, 118 aa, Synthetic construct
QVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDETDYALDYWGQGTLVTVSS

SEQ ID NO: 122 - protein, 107 aa, Synthetic construct
DIQMTQSPSSLSASVGDRVTITCQASQDISNYLNWYQQKPGKAPKLLIYDASNLETGVPSRFSGSGSGTDFTFTISSLQPEDIATYYCQQYEDLPLTFGGGTKVEIK

SEQ ID NO: 123 - protein, 8 aa, Synthetic construct
GFTFDDYG

SEQ ID NO: 124 - protein, 8 aa, Synthetic construct
INWEGGST

SEQ ID NO: 125 - protein, 18 aa, Synthetic construct
ARDEQLGGNYYYYYYMDV

SEQ ID NO: 126 - protein, 6 aa, Synthetic construct
QGIRND

SEQ ID NO: 127 - skipped

SEQ ID NO: 128 - protein, 9 aa, Synthetic construct
LQYNSYPLT

SEQ ID NO: 129 - protein, 5 aa, Synthetic construct
DYGMS

SEQ ID NO: 130 - protein, 17 aa, Synthetic construct
GINWEGGSTGYADSVKG

SEQ ID NO: 131 - protein, 16 aa, Synthetic construct
DEQLGGNYYYYYYMDV

SEQ ID NO: 132 - protein, 11 aa, Synthetic construct
RASQGIRNDLG

SEQ ID NO: 133 - protein, 7 aa, Synthetic construct
TASNLQS

SEQ ID NO: 134 - protein, 7 aa, Synthetic construct
GFTFDDY

SEQ ID NO: 135 - protein, 6 aa, Synthetic construct
NWEGGS

SEQ ID NO: 136 - protein, 6 aa, Synthetic construct
DDYGMS

SEQ ID NO: 137 - protein, 13 aa, Synthetic construct
WVSGINWEGGSTG

SEQ ID NO: 138 - protein, 17 aa, Synthetic construct
ARDEQLGGNYYYYYYMD

SEQ ID NO: 139 - protein, 7 aa, Synthetic construct
RNDLGWY

SEQ ID NO: 140 - protein, 10 aa, Synthetic construct
RLIYTASNLQ

SEQ ID NO: 141 - protein, 8 aa, Synthetic construct
LQYNSYPL

SEQ ID NO: 142 - protein, 125 aa, Synthetic construct
EVQLVESGGGVVRPGGSLRLSCAASGFTFDDYGMSWVRQAPGKGLEWVSGINWEGGSTGYADSVKGRFTISRDNAKNSLYLQMNSLRAEDTALYYCARDEQLGGNYYYYYYMDVWGKGTTVTVSS

SEQ ID NO: 143 - protein, 107 aa, Synthetic construct
DIQMTQSPSSLSASVGDRVTITCRASQGIRNDLGWYQQKPGKAPKRLIYTASNLQSGVPSRFSGSGSGTEFTLTISSLQPEDFATYYCLQYNSYPLTFGGGTKVEIK

SEQ ID NO: 144 - protein, 10 aa, Synthetic construct
ARDENYALDW

SEQ ID NO: 145 - protein, 8 aa, Synthetic construct
DENYALDW

SEQ ID NO: 146 - protein, 9 aa, Synthetic construct
ARDENYALD

SEQ ID NO: 147 - protein, 9 aa, Synthetic construct
QHYEDYPLT

SEQ ID NO: 148 - protein, 8 aa, Synthetic construct
QHYEDYPL

SEQ ID NO: 149 - protein, 117 aa, Synthetic construct
QVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDENYALDWWGQGTLVTVSS

SEQ ID NO: 150 - protein, 107 aa, Synthetic construct
DIQMTQSPSSLSASVGDRVTITCQASQDISNYLNWYQQKPGKAPKLLIYDASNLETGVPSRFSGSGSGTDFTFTISSLQPEDIATYYCQHYEDYPLTFGGGTKVEIK

SEQ ID NO: 151 - protein, 117 aa, Synthetic construct
EVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDENYALDWWGQGTLVTVSS

(19) United States
(12) Patent Application Publication
(10) Pub. No.: US 2025/0376511 A1
(43) Pub. Date: Dec. 11, 2025

(54) BINDING PROTEINS THAT TARGET aC1s, TfR, OR BOTH, AND COMPOSITIONS THEREOF

(71) Applicant: Genzyme Corporation, Cambridge, MA (US)

(72) Inventors: Cécile CAPDEVILA, Vitry (FR); Blandine DUPERRIER, Vitry (FR); Timothy HAMMOND, Cambridge, MA (US); Ayman ISMAIL, Cambridge, MA (US); Sagar KATHURIA, Cambridge, MA (US); Nina LEKSA, Cambridge, MA (US); Alessandro MASIERO, Vitry (FR); Sunghae PARK, Cambridge, MA (US); Yu QIU, Cambridge, MA (US); Anke STEINMETZ, Frankfurt am Main (DE); Serhan ZENGER, Cambridge, MA (US)

(73) Assignee: Genzyme Corporation, Cambridge, MA (US)

(21) Appl. No.: 19/229,314

(22) Filed: Jun. 5, 2025

Related U.S. Application Data
(60) Provisional application No. 63/656,433, filed on Jun. 5, 2024.

(51) Int. Cl. C07K 16/18; A61K 39/00; C07K 16/28

(52) U.S. Cl. CPC C07K 16/18; C07K 16/2881; A61K 2039/505; C07K 2317/31; C07K 2317/92

(57) ABSTRACT

The present disclosure provides binding proteins that target activated C1s (aC1s), as well as bispecific binding proteins that target aC1s and a central nervous system protein (e.g., transferrin receptor 1). Also provided is the use of these binding proteins to treat neurological complement-mediated disorders.

### CROSS-REFERENCE TO RELATED APPLICATION

This application claims priority to U.S. Provisional Patent Application No. 63/656,433, filed Jun. 5, 2024. The disclosure of that priority application is incorporated by reference herein in its entirety.

### SEQUENCE LISTING

The instant application contains a Sequence Listing which has been submitted electronically in XML format and is hereby incorporated by reference herein in its entirety. The electronic copy of the Sequence Listing, created on May 23, 2025, is named 122548.US037.xml and is 137,563 bytes in size.

### BACKGROUND OF THE INVENTION

Innate immunity via the complement cascade enables clearance of pathogens or damaged cells via phagocytosis. However, dysregulated complement cascade can cause deleterious inflammation. There are three pathways of initiation of the complement cascade—the classical pathway, the lectin pathway, and the alternative pathway. The classical pathway is initiated by activation of the C1 complex (C1q, C1r, and C1s). Upon binding to IgG or IgM immune complexes, C1q undergoes a conformational change, leading to C1r cleavage of C1s to its activated form (aC1s).

aC1s cleaves C4 and C2, which assemble to form C4b2a, a C3 convertase. All C3 convertases cleave C3 into the anaphylatoxin C3a and the opsonin C3b. Covalently attached C3b mediates phagocytosis of the opsonin-tagged cell. In addition, opsonized C3b amplifies the complement response through the alternative pathway, regardless of the initiation pathway. This amplification triggers the activation of the terminal pathway through the formation of C5 convertases, which cleave C5 into C5a, a potent anaphylatoxin, and C5b, a component of C5b9 or the membrane attack complex (MAC), a large pore complex that can cause cell lysis.

Aberrant activation of the classical complement pathway is linked to the development of autoimmune and inflammatory disorders, infectious diseases, and cancer. One therapeutic goal in treating such disorders is to inhibit the classical complement pathway, for example, by inhibiting aC1s. Inhibiting the classical complement pathway in the brain may treat complement-mediated neurological disorders. However, many therapeutic agents meet difficulties in crossing the blood-brain barrier (BBB), an endothelial cell barrier that limits the passage of molecules from the blood to the brain.

Transferrin receptor 1 (TfR), also known as CD71, is a ubiquitously expressed transmembrane glycoprotein involved in cellular uptake of iron. TfR imports iron through receptor-mediated endocytosis of transferrin, an iron-binding protein. Since TfR is highly expressed by brain capillary endothelial cells forming the blood-brain barrier (BBB) and transports iron across the BBB through transcytosis, it has been explored as a potential target for molecular shuttles that are designed to transport large molecule drugs across the BBB (see, e.g., Bourassa et al., Mol Pharm. (2019) 16(2):583-94).

In view of the role of the classical complement pathway in disease, there remains a need for aC1s-targeting therapies for treatment of complement-mediated disorders, and for delivery of such therapies to the brain for treatment of neurological complement-mediated disorders.

### SUMMARY OF THE INVENTION

The present disclosure provides an aC1s-binding protein comprising an anti-aC1s binding domain that comprises:
a) a heavy chain variable region (VH) comprising heavy chain complementarity-determining regions (HCDR) 1-3 set forth in SEQ ID NOs: 1, 2, and 3, respectively; and a light chain variable region (VL) comprising light chain CDR (LCDR) 1-3 set forth in SEQ ID NOs: 4, 7 and 8, respectively; or
b) a VH comprising HCDR1-3 set forth in SEQ ID NOs: 1, 2, and 3, respectively; and a VL comprising LCDR1-3 set forth in SEQ ID NOs: 5, 7 and 8, respectively.
   In some embodiments, the VH comprises any one of SEQ ID NOs: 9-12, and the VL comprises any one of SEQ ID NOs: 13-18. For example, the VH and VL may comprise:
SEQ ID NOs: 9 and 13, respectively;
SEQ ID NOs: 9 and 15, respectively;
SEQ ID NOs: 9 and 17, respectively;
SEQ ID NOs: 10 and 14, respectively;
SEQ ID NOs: 10 and 16, respectively;
SEQ ID NOs: 10 and 18, respectively;
SEQ ID NOs: 11 and 13, respectively;
SEQ ID NOs: 11 and 15, respectively;
SEQ ID NOs: 11 and 17, respectively;
SEQ ID NOs: 12 and 14, respectively;
SEQ ID NOs: 12 and 16, respectively; or
SEQ ID NOs: 12 and 18, respectively.

In some embodiments, the aC1s-binding protein herein has at least one property selected from
a) binds to human aC1s with a K_{D} of 1-5 nM as determined by surface plasmon resonance (SPR);
b) binds to cynomolgus aC1s with a K_{D} of 0.1-0.5 nM as determined by SPR;
c) inhibits complement in vitro as determined by a Wieslab classical complement pathway assay; or
d) any combination (e.g., all) of a)-c).

In some embodiments, the aC1s-binding protein herein is a monoclonal antibody or an antigen-binding fragment thereof. In certain embodiments, the aC1s-binding protein is an antigen-binding fragment comprising a Fab, Fab′, F(ab′)₂, or scFv.

In some embodiments, the aC1s-binding protein herein is fused to a cell-penetrating peptide that binds a central nervous system (CNS) target. In some embodiments, the aC1s-binding protein herein comprises an Fc region with one or both chains modified to bind a CNS target, and may be a bivalent anti-aC1s antibody or antigen-binding fragment thereof wherein one chain of the Fc region is modified to bind the CNS target. In certain embodiments, the CNS target is an endothelial cell receptor of the blood-brain barrier (BBB), such as transferrin receptor 1 (TfR).

The present disclosure also provides a TfR-binding protein comprising an anti-TfR binding domain that comprises:
a VH comprising HCDR1-3 set forth in SEQ ID NOs: 22, 23, and 24, respectively; and
a VL comprising LCDR1-3 set forth in SEQ ID NOs: 25, 27, and 28, respectively.
   In some embodiments, the VH comprises SEQ ID NO: 29 or 30, and the VL comprises any one of SEQ ID NOs: 31-34. For example, the VH and VL may comprise:
SEQ ID NOs: 29 and 31, respectively;
SEQ ID NOs: 29 and 33, respectively;
SEQ ID NOs: 30 and 32, respectively; or
SEQ ID NOs: 30 and 34, respectively.

In some embodiments, the TfR-binding protein herein has at least one property selected from
a) binds to human TfR with a K_{D} of 1-50 nM as determined by surface plasmon resonance (SPR);
b) binds to cynomolgus TfR with a K_{D} of 30-90 nM as determined by SPR; or
c) a) and b).
   In particular embodiments, the TfR-binding protein has both properties a) and b).

In some embodiments, the TfR-binding protein herein is a monoclonal antibody or an antigen-binding fragment thereof. In certain embodiments, the TfR-binding protein is an antigen-binding fragment comprising a Fab, Fab′, F(ab′)₂, or scFv.

The aC1s-binding protein or TfR-binding protein herein may be an antibody of human isotype subclass IgG1, IgG2, IgG3, or IgG4. In some embodiments, the antibody comprises
a) a human IgG1 or IgG4 constant region;
b) a human kappa light chain constant region; or
c) both a) and b).

In some embodiments, the anti-aC1s or anti-TfR antibody herein comprises a human IgG4 constant region that may comprise mutations selected from
i) S228P,
ii) L235E,
iii) M428L and N434S,
iv) H435R and Y436F, or
v) any combination of i)-iv),
   wherein the mutation positions are according to Eu numbering. In certain embodiments, the antibody may comprise a first heavy chain constant region that comprises the mutations of i)-iii), and a second heavy chain constant region that comprises the mutations of i)-iv). In some embodiments, the antibody comprises a human IgG4 heavy chain constant region that comprises SEQ ID NO: 40, optionally without the C-terminal lysine.

In some embodiments, the anti-aC1s or anti-TfR antibody herein comprises a human IgG1 constant region that may comprise mutations selected from
i) L234A and L235A,
ii) A237G, P329A, A330S, and P331S,
iii) M428L and N434S,
iv) H435R and Y436F, and
v) any combination of i)-iv),
   wherein the mutation positions are according to Eu numbering. In certain embodiments, the antibody may comprise a first heavy chain that comprises the mutations of i)-iii), and a second heavy chain that comprises the mutations of i)-iv). In some embodiments, the antibody comprises a human IgG1 heavy chain constant region that comprises any one of SEQ ID NOs: 37-39, optionally without the C-terminal lysine if present.

The present disclosure also provides a bispecific binding protein comprising
a) an anti-aC1s binding domain, and
b) a binding domain specific for a CNS target.
   In some embodiments, the anti-aC1s binding domain competes for binding with, or binds to the same epitope as, the anti-aC1s binding domain of an aC1s-binding protein described above. In some embodiments, the anti-aC1s binding domain competes for binding with, or binds to the same epitope as, the anti-aC1s binding domain of an aC1s-binding protein comprising a VH and a VL that comprise
a) SEQ ID NOs: 100 and 101, respectively;
b) SEQ ID NOs: 121 and 122, respectively;
c) SEQ ID NOs: 142 and 143, respectively;
d) SEQ ID NOs: 149 and 150, respectively; or
e) SEQ ID NOs: 151 and 150, respectively.
   In some embodiments, the anti-aC1s binding domain of a bispecific binding protein herein comprises HCDR1-3 and LCDR1-3 set forth in
SEQ ID NOs: 1, 2, 3, 4, 7, and 8, respectively;
SEQ ID NOs: 1, 2, 3, 5, 7, and 8, respectively;
SEQ ID NOs: 1, 2, 3, 6, 7, and 8, respectively;
SEQ ID NOs: 81, 82, 83, 84, 85, and 86, respectively;
SEQ ID NOs: 102, 103, 104, 105, 106, and 107, respectively;
SEQ ID NOs: 123, 124, 125, 126, 127, and 128, respectively;
SEQ ID NOs: 102, 103, 144, 105, 106, and 147, respectively; or
SEQ ID NOs: 102, 103, 144, 105, 106, and 148, respectively.
   In some embodiments, the anti-aC1s binding domain comprises VH and VL that are set forth in, or are at least 90% identical to,
SEQ ID NOs: 9 and 13, respectively;
SEQ ID NOs: 9 and 15, respectively;
SEQ ID NOs: 9 and 17, respectively;
SEQ ID NOs: 9 and 19, respectively;
SEQ ID NOs: 9 and 20, respectively;
SEQ ID NOs: 10 and 14, respectively;
SEQ ID NOs: 10 and 16, respectively;
SEQ ID NOs: 10 and 18, respectively;
SEQ ID NOs: 10 and 21, respectively;
SEQ ID NOs: 11 and 13, respectively;
SEQ ID NOs: 11 and 15, respectively;
SEQ ID NOs: 11 and 17, respectively;
SEQ ID NOs: 11 and 19, respectively;
SEQ ID NOs: 11 and 20, respectively;
SEQ ID NOs: 12 and 14, respectively;
SEQ ID NOs: 12 and 16, respectively;
SEQ ID NOs: 12 and 18, respectively;
SEQ ID NOs: 12 and 21, respectively;
SEQ ID NOs: 100 and 101, respectively;
SEQ ID NOs: 121 and 122, respectively;
SEQ ID NOs: 142 and 143, respectively;
SEQ ID NOs: 149 and 150, respectively; or
SEQ ID NOs: 151 and 150, respectively.

In some embodiments, the binding domain of the bispecific binding protein that is specific for a CNS target binds to an endothelial cell receptor (ECR) of the blood brain barrier. The ECR may be, e.g., a transferrin receptor, insulin receptor, insulin-like growth factor receptor, low-density lipoprotein receptor, or folate receptor. In particular embodiments, the ECR is transferrin receptor 1 (TfR), and the binding domain specific for a CNS target is an anti-TfR binding domain. In some embodiments, the anti-TfR binding domain
a) binds to human TfR with a K_{D} of 10-50 nM (e.g., 25-45 nM),
b) binds to cynomolgus TfR with a K_{D} of 30-90 nM (e.g., 30-80 nM), or
c) a) and b).

In some embodiments, the anti-TfR binding domain of the bispecific binding protein herein competes for binding with, or binds to the same epitope as, the anti-TfR binding domain of a TfR-binding protein described above. In some embodiments, the anti-TfR binding domain of a bispecific binding protein herein comprises HCDR1-3 and LCDR1-3 set forth in
SEQ ID NOs: 22, 23, 24, 25, 27, and 28, respectively; or
SEQ ID NOs: 22, 23, 24, 26, 27, and 28, respectively.
   In some embodiments, the anti-TfR binding domain comprises VH and VL that are set forth in, or are at least 90% identical to,
SEQ ID NOs: 29 and 31, respectively;
SEQ ID NOs: 29 and 33, respectively;
SEQ ID NOs: 29 and 35, respectively;
SEQ ID NOs: 30 and 32, respectively;
SEQ ID NOs: 30 and 34, respectively; or
SEQ ID NOs: 30 and 36, respectively.

In some embodiments, a bispecific binding protein herein comprises
an anti-aC1s binding domain comprising HCDR1-3 and LCDR1-3 set forth in SEQ ID NOs: 1, 2, 3, 4, 7, and 8, respectively, and an anti-TfR binding domain comprising HCDR1-3 and LCDR1-3 set forth in SEQ ID NOs: 22, 23, 24, 25, 27, and 28, respectively;
an anti-aC1s binding domain comprising HCDR1-3 and LCDR1-3 set forth in SEQ ID NOs: 1, 2, 3, 4, 7, and 8, respectively, and an anti-TfR binding domain comprising HCDR1-3 and LCDR1-3 set forth in SEQ ID NOs: 22, 23, 24, 26, 27, and 28, respectively;
an anti-a1Cs binding domain comprising HCDR1-3 and LCDR1-3 set forth in SEQ ID NOs: 1, 2, 3, 5, 7, and 8, respectively, and an anti-TfR binding domain comprising HCDR1-3 and LCDR1-3 set forth in SEQ ID NOs: 22, 23, 24, 25, 27, and 28, respectively;
an anti-aC1s binding domain comprising HCDR1-3 and LCDR1-3 set forth in SEQ ID NOs: 1, 2, 3, 5, 7, and 8, respectively, and an anti-TfR binding domain comprising HCDR1-3 and LCDR1-3 set forth in SEQ ID NOs: 22, 23, 24, 26, 27, and 28, respectively;
an anti-aC1s binding domain comprising HCDR1-3 and LCDR1-3 set forth in SEQ ID NOs: 1, 2, 3, 6, 7, and 8, respectively, and an anti-TfR binding domain comprising HCDR1-3 and LCDR1-3 set forth in SEQ ID NOs: 22, 23, 24, 25, 27, and 28, respectively;
an anti-aC1s binding domain comprising HCDR1-3 and LCDR1-3 set forth in SEQ ID NOs: 1, 2, 3, 6, 7, and 8, respectively, and an anti-TfR binding domain comprising HCDR1-3 and LCDR1-3 set forth in SEQ ID NOs: 22, 23, 24, 26, 27, and 28, respectively;
an anti-aC1s binding domain comprising HCDR1-3 and LCDR1-3 set forth in SEQ ID NOs: 81, 82, 83, 84, 85, and 86, respectively, and an anti-TfR binding domain comprising HCDR1-3 and LCDR1-3 set forth in SEQ ID NOs: 22, 23, 24, 25, 27, and 28, respectively;
an anti-aC1s binding domain comprising HCDR1-3 and LCDR1-3 set forth in SEQ ID NOs: 81, 82, 83, 84, 85, and 86, respectively, and an anti-TfR binding domain comprising HCDR1-3 and LCDR1-3 set forth in SEQ ID NOs: 22, 23, 24, 26, 27, and 28, respectively;
an anti-aC1s binding domain comprising HCDR1-3 and LCDR1-3 set forth in SEQ ID NOs: 102, 103, 104, 105, 106, and 107, respectively, and an anti-TfR binding domain comprising HCDR1-3 and LCDR1-3 set forth in SEQ ID NOs: 22, 23, 24, 25, 27, and 28, respectively;
an anti-aC1s binding domain comprising HCDR1-3 and LCDR1-3 set forth in SEQ ID NOs: 102, 103, 104, 105, 106, and 107, respectively, and an anti-TfR binding domain comprising HCDR1-3 and LCDR1-3 set forth in SEQ ID NOs: 22, 23, 24, 26, 27, and 28, respectively;
an anti-aC1s binding domain comprising HCDR1-3 and LCDR1-3 set forth in SEQ ID NOs: 123, 124, 125, 126, 127, and 128, respectively, and an anti-TfR binding domain comprising HCDR1-3 and LCDR1-3 set forth in SEQ ID NOs: 22, 23, 24, 25, 27, and 28, respectively;
an anti-aC1s binding domain comprising HCDR1-3 and LCDR1-3 set forth in SEQ ID NOs: 123, 124, 125, 126, 127, and 128, respectively, and an anti-TfR binding domain comprising HCDR1-3 and LCDR1-3 set forth in SEQ ID NOs: 22, 23, 24, 26, 27, and 28, respectively;
an anti-aC1s binding domain comprising HCDR1-3 and LCDR1-3 set forth in SEQ ID NOs: 102, 103, 144, 105, 106, and 147, respectively, and an anti-TfR binding domain comprising HCDR1-3 and LCDR1-3 set forth in SEQ ID NOs: 22, 23, 24, 25, 27, and 28, respectively;
an anti-aC1s binding domain comprising HCDR1-3 and LCDR1-3 set forth in SEQ

ID NOs: 102, 103, 144, 105, 106, and 147, respectively, and an anti-TfR binding domain comprising HCDR1-3 and LCDR1-3 set forth in SEQ ID NOs: 22, 23, 24, 26, 27, and 28, respectively;
an anti-aC1s binding domain comprising HCDR1-3 and LCDR1-3 set forth in SEQ ID NOs: 102, 103, 144, 105, 106, and 148, respectively, and an anti-TfR binding domain comprising HCDR1-3 and LCDR1-3 set forth in SEQ ID NOs: 22, 23, 24, 25, 27, and 28, respectively; or
an anti-aC1s binding domain comprising HCDR1-3 and LCDR1-3 set forth in SEQ ID NOs: 102, 103, 144, 105, 106, and 148, respectively, and an anti-TfR binding domain comprising HCDR1-3 and LCDR1-3 set forth in SEQ ID NOs: 22, 23, 24, 26, 27, and 28, respectively.

In some embodiments, a bispecific binding protein herein comprises
an anti-aC1s binding domain comprising VH and VL set forth in SEQ ID NOs: 9 and 13, respectively, and an anti-TfR binding domain comprising VH and VL set forth in SEQ ID NOs: 29 and 31, respectively;
an anti-aC1s binding domain comprising VH and VL set forth in SEQ ID NOs: 11 and 13, respectively, and an anti-TfR binding domain comprising VH and VL set forth in SEQ ID NOs: 29 and 31, respectively;
an anti-aC1s binding domain comprising VH and VL set forth in SEQ ID NOs: 9 and 17, respectively, and an anti-TfR binding domain comprising VH and VL set forth in SEQ ID NOs: 29 and 31, respectively;
an anti-aC1s binding domain comprising VH and VL set forth in SEQ ID NOs: 11 and 17, respectively, and an anti-TfR binding domain comprising VH and VL set forth in SEQ ID NOs: 29 and 31, respectively;
an anti-aC1s binding domain comprising VH and VL set forth in SEQ ID NOs: 100 and 101, respectively, and an anti-TfR binding domain comprising VH and VL set forth in SEQ ID NOs: 29 and 31, respectively;
an anti-aC1s binding domain comprising VH and VL set forth in SEQ ID NOs: 121 and 122, respectively, and an anti-TfR binding domain comprising VH and VL set forth in SEQ ID NOs: 29 and 31, respectively;
an anti-aC1s binding domain comprising VH and VL set forth in SEQ ID NOs: 142 and 143, respectively, and an anti-TfR binding domain comprising VH and VL set forth in SEQ ID NOs: 29 and 31, respectively;
an anti-aC1s binding domain comprising VH and VL set forth in SEQ ID NOs: 149 and 150, respectively, and an anti-TfR binding domain comprising VH and VL set forth in SEQ ID NOs: 29 and 31, respectively; or
an anti-aC1s binding domain comprising VH and VL set forth in SEQ ID NOs: 151 and 150, respectively, and an anti-TfR binding domain comprising VH and VL set forth in SEQ ID NOs: 29 and 31, respectively.

The bispecific binding protein herein may comprise at least one property selected from
a) binds to human aC1s with a K_{D} of 1-10 nM (e.g., 1-5 nM) as determined by SPR;
b) binds to cynomolgus aC1s with a K_{D} of 0.1-1 nM (e.g., 0.1-0.5 nM) as determined by SPR;
c) binds to human TfR with a K_{D} of 10-50 nM (e.g., 25-45 nM) as determined by SPR;
d) binds to cynomolgus TfR with a K_{D} of 30-90 nM (e.g., 30-80 nM) as determined by SPR;
e) crosses the blood-brain barrier and achieves a higher maximal concentration in brain than a monospecific aC1s-binding protein comprising the anti-aC1s binding domain;
f) achieves a higher maximal concentration in the CSF than a monospecific aC1s-binding protein comprising the anti-aC1s binding domain;
g) inhibits neuronal complement deposition in vitro in a Wieslab classical complement pathway assay;
h) inhibits C3d deposition in vitro in iPSC tricultures; or
i) any combination (e.g., all) of a)-h).

In some embodiments, the bispecific binding protein herein is monovalent for aC1s and monovalent for the CNS target (e.g., TfR). In certain embodiments, the bispecific binding protein may comprise two heavy chains and two light chains, wherein one pair of heavy and light chains forms the anti-aC1s binding domain, and the other pair of heavy and light chains forms the anti-CNS target (e.g., TfR) binding domain, of the bispecific binding protein.

The present disclosure also provides a bispecific binding protein comprising
a) a TfR-binding protein described above or an anti-TfR binding domain thereof, and
b) a binding domain specific for another, distinct target protein.
   In some embodiments, the distinct target protein is a protein of the complement system. In certain embodiments, the distinct target protein is C1s, and may be activated C1s (aC1s).

The present disclosure also provides a bispecific binding protein comprising
a) an aC1s-binding protein described above or an anti-aC1s binding domain thereof, and
b) a binding domain specific for another, distinct target protein.
   In some embodiments, the distinct target protein is a CNS target protein, such as an endothelial cell receptor (ECR) of the blood brain barrier. The ECR may be, e.g., a transferrin receptor, insulin receptor, insulin-like growth factor receptor, low-density lipoprotein receptor, or folate receptor. In particular embodiments, the CNS target protein is TfR.

In some embodiments, the bispecific binding protein herein comprises an Fc region, and may be a bispecific antibody. The Fc region, or the antibody, may be of human isotype subclass IgG1, IgG2, IgG3, or IgG4. In certain embodiments, the bispecific binding protein comprises
a) a human IgG1 or IgG4 heavy chain constant region;
b) a human kappa light chain constant region; or
c) both a) and b).

In some embodiments, the bispecific binding protein comprises a human IgG1 or IgG4 heavy chain constant region and a human kappa light chain constant region, wherein the heavy chain constant region comprises T187E, K213E, and K218D mutations and the light chain constant region comprises S114A, D122K, E123K, and N137K mutations (Eu numbering).

In some embodiments, the bispecific binding protein herein comprises
a first heavy chain constant region that comprises one or more knob mutations, optionally wherein the knob mutations comprise S354C and T366W; and
a second heavy chain constant region that comprises one or more hole mutations, optionally wherein the hole mutations comprise Y349C, T366S, L368A, and Y407V (Eu numbering).

In some embodiments, the bispecific binding protein herein comprises a human IgG4 heavy chain constant region that comprises mutations selected from
i) S228P,
ii) L235E,
iii) M428L and N434S,
iv) H435R and Y436F, or
v) any combination of i)-iv)
(Eu numbering).
   In some embodiments, the bispecific binding protein comprises a first heavy chain constant region that comprises the mutations of i)-iii), and a second heavy chain constant region that comprises the mutations of i)-iv). In certain embodiments, the first heavy chain constant region further comprises knob mutations of S354C and T366W, and the second heavy chain constant region further comprises hole mutations of Y349C, T366S, L368A, and Y407V (Eu numbering), or vice-versa. The human IgG4 heavy chain constant region may comprise, e.g., any one of SEQ ID NOs: 40, 43, and 44, optionally without the C-terminal lysine if present. In some embodiments, the bispecific binding protein comprises two heavy chain constant regions that both comprise SEQ ID NO: 40; or a first heavy chain constant region that comprises SEQ ID NO: 43 and a second heavy chain constant region that comprises SEQ ID NO: 44.

In some embodiments, the bispecific binding protein herein comprises a human IgG1 heavy chain constant region that comprises mutations selected from
i) L234A and L235A,
ii) A237G, P329A, A330S, and P331S,
iii) M428L and N434S,
iv) H435R and Y436F, and
v) any combination of i)-iv)
(Eu numbering).
   In some embodiments, the bispecific binding protein comprises a first heavy chain constant region that comprises the mutations of i)-iii), and a second heavy chain constant region that comprises the mutations of i)-iv). In certain embodiments, the first heavy chain constant region further comprises knob mutations of S354C and T366W, and the second heavy chain constant region further comprises hole mutations of Y349C, T366S, L368A, and Y407V (Eu numbering), or vice-versa. The human IgG1 heavy chain constant region may comprise, e.g., any one of SEQ ID NOs: 37-39, 41, and 42, optionally without the C-terminal lysine if present. In some embodiments, the bispecific binding protein comprises two heavy chain constant regions that both comprise SEQ ID NO: 37, 38, or 39; or a first heavy chain constant region that comprises SEQ ID NO: 41 and a second heavy chain constant region that comprise SEQ ID NO: 42.

In some embodiments, the present disclosure provides a bispecific binding protein that binds to aC1s and TfR, comprising a first heavy chain that comprises SEQ ID NO: 47, a second heavy chain that comprises SEQ ID NO: 51, a first light chain that comprises SEQ ID NO: 49, and a second light chain that comprises SEQ ID NO: 52.

In some embodiments, the present disclosure provides a bispecific binding protein that binds to aC1s and TfR, comprising a first heavy chain that comprises SEQ ID NO: 47, a second heavy chain that comprises SEQ ID NO: 51, a first light chain that comprises SEQ ID NO: 50, and a second light chain that comprises SEQ ID NO: 52.

In some embodiments, the present disclosure provides a bispecific binding protein that binds to aC1s and TfR, comprising a first heavy chain that comprises SEQ ID NO: 48, a second heavy chain that comprises SEQ ID NO: 51, a first light chain that comprises SEQ ID NO: 49, and a second light chain that comprises SEQ ID NO: 52.

In some embodiments, the present disclosure provides a bispecific binding protein that binds to aC1s and TfR, comprising a first heavy chain that comprises SEQ ID NO: 48, a second heavy chain that comprises SEQ ID NO: 51, a first light chain that comprises SEQ ID NO: 50, and a second light chain that comprises SEQ ID NO: 52.

The present disclosure also provides a pharmaceutical composition comprising an aC1s-binding protein herein or a bispecific binding protein herein, and a pharmaceutically acceptable excipient.

The present disclosure also provides isolated nucleic acid molecule(s) encoding an aC1s-binding protein herein, a TfR-binding protein herein, or a bispecific binding protein herein. In some embodiments, the nucleic acid molecule(s) are expression constructs.

The present disclosure also provides a host cell comprising the isolated nucleic acid molecule(s) herein. In some embodiments, the host cell is a mammalian cell. Also provided is a method of producing an aC1s-binding protein, a TfR-binding protein, or a bispecific binding protein herein, comprising culturing the host cell under conditions that allow expression of the binding protein, and isolating the binding protein from the cell culture.

The present disclosure provides a method of treating a complement-mediated neurological disorder in a subject in need thereof (e.g., a mammalian subject such as a human subject), comprising administering a therapeutically effective amount of an aC1s-binding protein herein or a bispecific binding protein herein to the subject. Also provided is the use of an aC1s-binding protein herein or a bispecific binding protein herein for the manufacture of a medicament for treating a neurological complement-mediated disorder in a subject (e.g., a human subject) in need thereof. Also provided is an aC1s-binding protein herein or a bispecific binding protein herein for use in treating a complement-mediated neurological disorder in a subject (e.g., a human subject) in need thereof.

In some embodiments, the complement-mediated neurological disorder is amyotrophic lateral sclerosis (ALS), Alzheimer's disease (AD), Huntington's disease (HD), an autoimmune peripheral neuropathy, a neurodegenerative eye disease, or dementia. The dementia may be, e.g., frontotemporal dementia (FTD).

Other features, objectives, and advantages of the invention are apparent in the detailed description that follows. It should be understood, however, that the detailed description, while indicating embodiments and aspects of the invention, is given by way of illustration only, not limitation. Various changes and modification within the scope of the invention will become apparent to those skilled in the art from the detailed description.

### BRIEF DESCRIPTION OF THE FIGURES

FIGS. 1A and 1B are a set of line graphs showing the concentration of the indicated antibodies in the brain tissue of humanized TfR mice (FIG. 1A) or cerebrospinal fluid (CSF) and plasma of cynomolgus monkeys (FIG. 1B) at several timepoints following antibody injection.

FIG. 2 is a set of line graphs showing the concentration of the indicated antibodies in the brain tissue (top) or spinal cord (bottom) of humanized TfR mice at several timepoints following antibody injection.

FIG. 3 is a line graph showing the concentration of the indicated antibody variants in the brains of mice at 1 and 24 hours following antibody injection.

FIG. 4 is a line graph showing the degree of classical complement pathway (CCP) activation in the presence of the indicated antibodies at different concentrations as determined by the Wieslab Complement Pathway assay (in 1% NHS).

FIG. 5 is a line graph showing the inhibition of neuronal complement deposition (C3d deposition) after incubation with the indicated antibodies in induced pluripotent stem cell tricultures stimulated with 6% complement preserved serum.

FIG. 6 is a set of line graphs showing the concentration of the indicated antibody variants in the brain (top) or plasma (bottom) of humanized TfR mice at 1, 24, and 48 hours following antibody injection.

FIG. 7 is a line graph showing the degree of classical complement pathway (CCP) activation in the Wieslab Complement Pathway assay (in 1% NHS) in the presence of the indicated antibodies at different concentrations.

FIG. 8 is a line graph showing the degree of classical complement pathway (CCP) activation in the Wieslab Complement Pathway assay in the presence of the indicated antibodies at different concentrations.

FIG. 9 is a set of line graphs showing the concentration of the indicated antibodies in the brain (top) or plasma (bottom) of humanized TfR mice dosed at 3, 10, and 30 mg/kg at several timepoints following antibody injection.

FIG. 10 is a set of line graphs showing the concentration of the bispecific anti-aC1s (L4)/anti-TfR (v6) IgG4 antibody in various tissues of cynomolgus monkeys at several timepoints after dosing at 10 mg/kg or 30 mg/kg.

FIGS. 11A and 11B are a set of images (FIG. 11A) showing the cellular co-localization of C3d with neurons (Tuj1) in induced pluripotent stem cell brain tricultures following treatment with an anti-aC1s/anti-TfR bispecific antibody, and a line graph (FIG. 11B) showing the percent of C3d fluorescence colocalizing with neurons resulting from treatment with different concentrations of an anti-aC1s/anti-TfR bispecific antibody.

### DETAILED DESCRIPTION OF THE INVENTION

The present disclosure provides isolated binding proteins, such as antibodies and antigen-binding fragments thereof, that bind the activated form of C1s (aC1s), or that bind to transferrin 1 receptor (TfR).

The present disclosure also provides multispecific (e.g., bispecific) binding proteins that pair an anti-aC1s binding domain with a domain that binds to a CNS target (e.g., an epithelial cell receptor (ECR) of the BBB, such as transferrin receptor 1 (TfR)). The domain that binds to the CNS target may facilitate transport of the anti-aC1s binding domain to the CNS, e.g., across the BBB.

The present disclosure also provides multispecific (e.g., bispecific) binding proteins that pair an anti-TfR binding domain with a domain that binds to a target (e.g., a protein of the complement system, such as aC1s). The anti-TfR binding domain may facilitate transport of the target-binding domain to the CNS, e.g., across the BBB.

Unless otherwise indicated, aC1s herein refers to human aC1s, and TfR herein refers to human TfR. A human C1s polypeptide sequence is available under UniProt Accession No. P09871 (SEQ ID NO: 53). A human TfR polypeptide sequence is available under UniProt Accession No. P02786 (SEQ ID NO: 54).

### I. Binding Proteins

The present disclosure provides aC1s-binding proteins and TfR-binding proteins, such as antibodies or antigen-binding fragments thereof. The term “antibody” herein includes monospecific and multispecific (e.g., bispecific) antibodies. “Antibody” (Ab) or “immunoglobulin” (Ig), as used herein, may refer to a tetramer comprising two heavy (H) chains and two light (L) chains interconnected by disulfide bonds. Each heavy chain is comprised of a heavy chain variable region or domain (VH) and a heavy chain constant region (CH). Each light chain is composed of a light chain variable region or domain (VL) and a light chain constant region (CL). The VH and VL domains can be subdivided further into regions of hypervariability, termed “complementarity-determining regions” (CDRs), interspersed with regions that are more conserved, termed “framework regions” (FRs). Each VH and VL is composed of three CDRs (HCDR herein designates a CDR from the heavy chain; and LCDR herein designates a CDR from the light chain) and four FRs, arranged from amino-terminus to carboxyl-terminus in the following order: FR1, CDR1, FR2, CDR2, FR3, CDR3, and FR4.

The precise amino acid sequence boundaries of a given CDR or FR can be defined by several well-known systems, including those described by Kabat et al., 5th Ed., Public Health Service, National Institutes of Health, Bethesda, MD (1991) (“Kabat” system); Al-Lazikani et al., J Mol Biol. (1997) 273:927-48) (“Chothia” system); MacCallum et al., J Mol Biol. (1996) 262:732-45 (“contact” system); Lefranc et al., Dev Comp Immunol. (2003) 27(1):55-77 (“IMGT” system); Honegger and Plückthun, J Mol Biol. (2001) 309(3):657-70 (“Aho” system); and Whitelegg and Rees, Protein Eng. (2000) 13(12):819-24 (“AbM” system). The boundaries of a given CDR or FR may vary depending on the system used. For example, the Kabat system is based on sequence alignments, while the Chothia system is based on structural information. Numbering for both the Kabat and Chothia systems is based upon the most common antibody region sequence lengths, with insertions accommodated by insertion letters, for example, “30a.” The two systems place certain insertions and deletions (“indels”) at different positions, resulting in differential numbering. The contact system is based on analysis of complex crystal structures and is similar in many respects to the Chothia system. The CDRs of the antibodies described herein can be defined, e.g., by a system selected from Kabat, Chothia, IMGT, Aho, AbM, or combinations thereof.

The antibodies provided herein may be of any immunoglobulin isotype, such as IgG (e.g., IgG1, IgG2, IgG3, or IgG4). The antibodies preferably comprise a human IgG (e.g., IgG1 or IgG4) heavy chain constant region. In some embodiments, the IgG heavy chain constant region may comprise mutations that improve the therapeutic potential of the antibody, such as mutations that reduce or eliminate effector functions of the antibody (see, e.g., Wang et al., Protein Cell (2018) 9(1):63-73). For example, the antibody may comprise a human IgG1 heavy chain constant region with the mutation(s) L235E or L234A/L235A (“LALA” mutations); M252Y/S254T/T256E (“YTE” mutations); and/or S298N/T299A/Y300S (“NNAS” mutations); in any combination. Further, for example, the monospecific or multispecific antibody herein may comprise a human IgG4 heavy chain constant region with the mutation L235E and/or the mutation S228P. In some embodiments, the IgG heavy chain constant region may comprise mutations that improve the serum half-life of the antibody, such as the M428L and/or N434S mutations (“LS” mutations). In some embodiments, the IgG heavy chain constant region may comprise mutations that improve manufacturing and yield of the antibody, such as H435R and Y436F mutations (“RF” mutations), which reduce binding to protein A and thus are advantageous for antibody purification. The IgG heavy chain constant region may also comprise knob-in-hole mutations (see, e.g., the descriptions herein).

In any embodiments of constant regions herein, an IgG heavy chain constant region, in combination with a light chain constant region, may additionally or alternatively comprise CR3/NN3 charge-pair mutations that facilitate specific heavy and light chain pairing (CR3: T187E mutation in the heavy chain constant region and N137K/S114A mutations in the light chain constant region; NN3: K213E and K218D mutations in the heavy chain constant region and E123K and D122K mutations in the light chain constant region).

Human constant regions with mutation(s) as described above are still considered “human” constant regions herein. Unless otherwise indicated, all residue numbers in IgG heavy and light chain constant regions are Eu numbers.

In certain embodiments, the monospecific or multispecific antibody herein comprises a human IgG4 heavy chain constant region comprising mutation(s) selected from
a) S228P,
b) L235E,
c) M428L and N434S,
d) H435R and Y436F, or
e) any combination of a)-d).

In certain embodiments, the monospecific or multispecific antibody herein comprises a first human IgG4 heavy chain constant region comprising S228P, L235E, M428L, and N434S mutations and a second human IgG4 heavy chain constant region comprising S228P, L235E, M428L, N434S, H435R, and Y436F mutations. In particular embodiments, e.g., of a multispecific antibody herein, the first and second human IgG4 heavy chain constant regions may also comprise knob-in-hole mutations, e.g., as described below. For example, one of the human IgG4 heavy chain constant regions (e.g., the first constant region, with mutations as described above) may comprise knob mutations of S354C and T336W, and the other human IgG4 heavy chain constant region (e.g., the second constant region, with mutations as described above) may comprise hole mutations of Y349C, T366S, L368A, and Y407V.

In certain embodiments, the monospecific or multispecific antibody herein comprises a human IgG1 heavy chain constant region comprising mutation(s) selected from
a) L234A and L235A,
b) A237G, P329A, A330S, and P331S,
c) M428L and N434S,
d) H435R and Y436F, and
e) any combination of a)-d).

In certain embodiments, the monospecific or multispecific antibody herein comprises a first human IgG1 heavy chain constant region comprising L234A, L235A, A327G, P329A, A330S, P331S, M428L, and N434S mutations and a second human IgG1 heavy chain constant region comprising L234A, L235A, A327G, P329A, A330S, P331S, M428L, N434S, H435R, and Y436F mutations.

In certain embodiments, e.g., of a multispecific antibody herein, the first and second human IgG1 heavy chain constant regions herein may also comprise knob-in-hole mutations, e.g., as described herein. Exemplary knob mutations may comprise S354C and/or T336W. Exemplary hole mutations may comprise Y349C, T366S, L368A, Y407V, or any combination thereof. For example, one of the human IgG1 heavy chain constant regions (e.g., the first constant region, with mutations as described above) may comprise knob mutations of S354C and T336W, and the other human IgG1 heavy chain constant region (e.g., the second constant region, with mutations as described above) may comprise hole mutations of Y349C, T366S, L368A, and Y407V.

In particular embodiments, the monospecific or multispecific antibody herein comprises a human IgG4 heavy chain constant region comprising any one of SEQ ID NOs: 40, 43, and 44, or a human IgG1 heavy chain constant region comprising any one of SEQ ID NOs: 37, 38, 39, 41, and 42, or said constant region sequence without the C-terminal lysine if present. The monospecific or multispecific antibody may comprise, e.g.,
two human IgG4 heavy chain constant regions both comprising SEQ ID NO: 40 (and optionally a human kappa light chain constant region comprising SEQ ID NO: 45);
a first human IgG4 heavy chain constant region comprising SEQ ID NO: 43 and a second human IgG4 heavy chain constant region comprising SEQ ID NO: 44 (and optionally a human kappa light chain constant region comprising SEQ ID NO: 46),
two human IgG1 heavy chain constant regions both comprising SEQ ID NO: 37, 38, or 39 (and optionally a human kappa light chain constant region comprising SEQ ID NO: 45); or
a first human IgG1 heavy chain constant region comprising SEQ ID NO: 41 and a second human IgG1 heavy chain constant region comprising SEQ ID NO: 42 (and optionally a human kappa light chain constant region comprising SEQ ID NO: 46).

In some embodiments, the binding proteins herein are antigen-binding fragments of full (tetrameric) antibodies. The term “antigen-binding fragment” or “antigen-binding portion” herein encompasses genetically engineered and/or otherwise modified forms of immunoglobulins that do not have the conventional full-length tetrameric structure. The term encompasses intrabodies, peptibodies, diabodies, triabodies, tetrabodies, Fv, Fab, Fab′, Fab′-SH, F(ab′)₂, single-chain antibody molecules (e.g., scFv or sFv), tandem di-scFv, and tandem tri-scFv.

The present aC1s-and TfR-binding proteins bind specifically to their targets (i.e., human aC1s and human TfR, respectively). “Specifically” herein indicates that the binding proteins bind to their target with an affinity described herein or higher. In addition, to facilitate pre-clinical studies in non-human primate (NHP) animal models, the aC1s-binding proteins and/or the TfR-binding proteins may have a suitable affinity for cynomolgus aC1s (caC1s) and/or cynomolgus TfR (cTfR), respectively. Several techniques can be used to characterize target binding affinity (K_{D}), such as surface plasmon resonance (SPR, using, e.g., BIAcore™) or bio-layer interferometry (BLI, using, e.g., Octet™ from ForteBio). Flow cytometry assays (e.g., FACS) using cells expressing membrane-bound targets can also be used to determine EC₅₀ or IC₅₀ values of the binding proteins; these values are indicative of binding to the targets in their native conformation.

### A. aC1s-Binding Proteins

In some embodiments, the binding proteins of the present disclosure bind to activated C1s (aC1s). In certain embodiments, the binding proteins are aC1s-binding proteins comprising anti-aC1s binding domains, such as anti-aC1s antibodies or antigen-binding fragments thereof.

In some embodiments, the anti-aC1s binding domain herein comprises HCDR1-3 and LCDR1-3 set forth in
SEQ ID NOs: 1, 2, 3, 4, 7, and 8, respectively; or
SEQ ID NOs: 1, 2, 3, 5, 7, and 8, respectively.

In some embodiments, the anti-aC1s binding domain herein comprises the HCDR1-3 in a VH comprising any one of SEQ ID NOs: 9-12 and the LCDR1-3 in a VL comprising any one of SEQ ID NOs: 13-18. The assignment of CDR regions may be in accordance with any method known in the art, such as IMGT®, Kabat, Chothia, Martin, Contact, or AHo definitions, or any combination of any of these definitions (Kabat plus Chothia, for example). Examples of CDR definitions under different methods are shown below for the VH and VL of exemplified anti-aC1s antibody aC1s-D32E:

HCDRs (aC1s-D32E)

| Definition | HCDR1 | SEQ | HCDR2 | SEQ | HCDR3 | SEQ |
|---|---|---|---|---|---|---|
| IMGT® | GFNIKDDY | 1 | IDPADGHT | 2 | ARYGYGREVFDY | 3 |
| Kabat | DDYIH | 55 | RIDPADGHTKYAPKFQV | 56 | YGYGREVFDY | 57 |
| Chothia | GFNIKDD | 60 | DPADGH | 61 | YGYGREVFDY | 57 |
| Contact | KDDYIH | 62 | WIGRIDPADGHTK | 63 | ARYGYGREVFD | 64 |

LCDRs (aC1s-D32E)

| Definition | LCDR1 | SEQ | LCDR2 | SEQ | LCDR3 | SEQ |
|---|---|---|---|---|---|---|
| IMGT® | QSVDYEGDSY | 4 | DAS | 7 | QQSNEDPWT | 8 |
| Kabat | KSSQSVDYEGDSYLN | 58 | DASNLES | 59 | QQSNEDPWT | 8 |
| Chothia | KSSQSVDYEGDSYLN | 58 | DASNLES | 59 | QQSNEDPWT | 8 |
| Contact | DYEGDSYLNWY | 65 | LLIYDASNLE | 66 | QQSNEDPW | 67 |

SEQ: SEQ ID NO:

Thus, for example, the HCDR1-3 sequences of SEQ ID NOs: 1, 2, and 3, respectively, may be replaced in any embodiment described herein by
SEQ ID NOs: 55, 56, and 57, respectively;
SEQ ID NOs: 60, 61, and 57, respectively; or
SEQ ID NOs: 62, 63, and 64, respectively.

Similarly, additionally or alternatively, the LCDR1-3 sequences of SEQ ID NOs: 4, 7, and 8, respectively, may be replaced in any embodiment described herein by
SEQ ID NOs: 58, 59, and 6, respectively; or
SEQ ID NOs: 65, 66, and 67, respectively.

Also contemplated is a set of CDRs specified according to any combination of the methods for defining CDRs shown above (e.g., HCDR1 may be defined by the Kabat method, HCDR2 may be defined by the IMGT® method, etc.). These methods or combinations of methods may be used to define the CDRs in the VH or VL of any binding domain herein.

In some embodiments, the anti-aC1s binding domain herein comprises a VH set forth in SEQ ID NO: 10 and a VL set forth in any one of SEQ ID NOs: 14, 16, and 18, in any combination. In some embodiments, the anti-aC1s binding domain herein comprises a VH set forth in SEQ ID NO: 12 and a VL set forth in any one of SEQ ID NOs: 14, 16, and 18, in any combination.

In some embodiments, the anti-aC1s binding domain comprises a VH and a VL with charge mutations to facilitate correct VH/VL pairing, such as in a multispecific context. In anti-aC1s VH and VL sequences herein, such charge mutations may appear in, e.g., VH/VL pairs wherein the VH comprises a Q39E mutation and the VL comprises a Q42K mutation. In some embodiments, the anti-aC1s binding domain herein comprises a VH set forth in SEQ ID NO: 9 and a VL set forth in any one of SEQ ID NOs: 13, 15, or 17, in any combination. In some embodiments, the anti-aC1s binding domain herein comprises a VH set forth in SEQ ID NO: 11 and a VL set forth in any one of SEQ ID NOs: 13, 15, or 17, in any combination.

In certain embodiments, the anti-aC1s binding domain comprises a VH and a VL set forth in SEQ ID NOs: 9 and 13, respectively.

In certain embodiments, the anti-aC1s binding domain comprises a VH and a VL set forth in SEQ ID NOs: 9 and 15, respectively.

In certain embodiments, the anti-aC1s binding domain comprises a VH and a VL set forth in SEQ ID NOs: 9 and 17, respectively.

In certain embodiments, the anti-aC1s binding domain comprises a VH and a VL set forth in SEQ ID NOs: 10 and 14, respectively.

In certain embodiments, the anti-aC1s binding domain comprises a VH and a VL set forth in SEQ ID NOs: 10 and 16, respectively.

In certain embodiments, the anti-aC1s binding domain comprises a VH and a VL set forth in SEQ ID NOs: 10 and 18, respectively.

In certain embodiments, the anti-aC1s binding domain comprises a VH and a VL set forth in SEQ ID NOs: 11 and 13, respectively.

In certain embodiments, the anti-aC1s binding domain comprises a VH and a VL set forth in SEQ ID NOs: 11 and 15, respectively.

In certain embodiments, the anti-aC1s binding domain comprises a VH and a VL set forth in SEQ ID NOs: 11 and 17, respectively.

In certain embodiments, the anti-aC1s binding domain comprises a VH and a VL set forth in SEQ ID NOs: 12 and 14, respectively.

In certain embodiments, the anti-aC1s binding domain comprises a VH and a VL set forth in SEQ ID NOs: 12 and 16, respectively.

In certain embodiments, the anti-aC1s binding domain comprises a VH and a VL set forth in SEQ ID NOs: 12 and 18, respectively.

The present disclosure provides an aC1s-binding protein comprising an anti-aC1s binding domain herein. In some embodiments, the aC1s-binding protein is an anti-aC1s antibody or an antigen-binding fragment thereof. The anti-aC1s antibody may comprise any heavy and light chain constant regions described herein. In some embodiments, the anti-aC1s antibody comprises a human IgG4 heavy chain constant region, optionally with mutations as described herein. For example, the human IgG4 heavy chain constant region may comprise any one of SEQ ID NOs: 40, 43, and 44 (optionally without the C-terminal lysine, if present). In some embodiments, the antibody comprises a first heavy chain constant region comprising SEQ ID NO: 43 and a second heavy chain constant region comprising SEQ ID NO: 44. In some embodiments, the anti-aC1s antibody comprises a human IgG1 heavy chain constant region, optionally with mutations as described herein. For example, the human IgG1 heavy chain constant region may comprise any one of SEQ ID NOs: 37-39, 41, and 42 (optionally without the C-terminal lysine, if present). In some embodiments, the antibody comprises a first heavy chain constant region comprising SEQ ID NO: 41 and a second heavy chain constant region comprising SEQ ID NO: 42. In some embodiments, the anti-aC1s antibody comprises a human kappa or lambda light chain constant region. In certain embodiments, the anti-aC1s antibody comprises a human kappa light chain constant region, e.g., comprising SEQ ID NO: 45 or 46. In particular embodiments, the anti-aC1s antibody comprises a human IgG4 heavy chain constant region comprising SEQ ID NO: 40 or a human IgG1 heavy chain constant region comprising any one of SEQ ID NOs: 37-39, and a human kappa light chain constant region comprising SEQ ID NO: 45. In particular embodiments, the anti-aC1s antibody comprises a human IgG4 heavy chain constant region comprising SEQ ID NO: 44 or a human IgG1 heavy chain constant region comprising SEQ ID NO: 42, and a human kappa light chain constant region comprising SEQ ID NO: 46.

In some embodiments, an anti-aC1s antibody herein may comprise
an HC comprising SEQ ID NO: 9 and any one of SEQ ID NOs: 37-40 and an LC comprising SEQ ID NO: 13 and SEQ ID NO: 45;
an HC comprising SEQ ID NO: 9 and any one of SEQ ID NOs: 37-40 and an LC comprising SEQ ID NO: 15 and SEQ ID NO: 45;
an HC comprising SEQ ID NO: 9 and any one of SEQ ID NOs: 37-40 and an LC comprising SEQ ID NO: 17 and SEQ ID NO: 45;
an HC comprising SEQ ID NO: 10 and any one of SEQ ID NOs: 37-40 and an LC comprising SEQ ID NO: 14 and SEQ ID NO: 45;
an HC comprising SEQ ID NO: 10 and any one of SEQ ID NOs: 37-40 and an LC comprising SEQ ID NO: 16 and SEQ ID NO: 45;
an HC comprising SEQ ID NO: 10 and any one of SEQ ID NOs: 37-40 and an LC comprising SEQ ID NO: 18 and SEQ ID NO: 45;
an HC comprising SEQ ID NO: 11 and any one of SEQ ID NOs: 37-40 and an LC comprising SEQ ID NO: 13 and SEQ ID NO: 45;
an HC comprising SEQ ID NO: 11 and any one of SEQ ID NOs: 37-40 and an LC comprising SEQ ID NO: 15 and SEQ ID NO: 45;
an HC comprising SEQ ID NO: 11 and any one of SEQ ID NOs: 37-40 and an LC comprising SEQ ID NO: 17 and SEQ ID NO: 45;
an HC comprising SEQ ID NO: 12 and any one of SEQ ID NOs: 37-40 and an LC comprising SEQ ID NO: 14 and SEQ ID NO: 45;
an HC comprising SEQ ID NO: 12 and any one of SEQ ID NOs: 37-40 and an LC comprising SEQ ID NO: 16 and SEQ ID NO: 45; or
an HC comprising SEQ ID NO: 12 and any one of SEQ ID NOs: 37-40 and an LC comprising SEQ ID NO: 18 and SEQ ID NO: 45.
   Also contemplated is any of the above HC and LC pairs wherein the HC is without the C-terminal lysine, if present. In any of the HC and LC pairs, the HC may further comprise “LS” mutations. Additionally or alternatively, in some embodiments, one HC may comprise “RF” mutations. Additionally or alternatively, in some embodiments, SEQ ID NO: 40 in the HC may be modified by S228P and/or L235E mutations.

Percent (%) sequence identity or homology with respect to a reference polypeptide sequence is the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways using available computer software. Appropriate parameters for aligning sequences are able to be determined, including algorithms needed to achieve maximal alignment over the full length of the sequences being compared. In some embodiments, the query sequence has at least 70% (e.g., at least 75, 80, 85, 90, or 95%) of the length of the reference sequence. For purposes herein, sequence homology or identity may be identified by BLAST, a bioinformatics program available at the server of the United States National Center for Biotechnology Information, using default parameters.

In some embodiments, the aC1s-binding proteins herein bind human aC1s with a K_{D} of no more than 5, 4.5, 4, 3.5, 3, 2.5, 2, 1.5, 1, 0.9, 0.8, or 0.7 nM as determined by SPR. In certain embodiments, the aC1s-binding proteins bind human aC1s with a K_{D} of 3.5 nM or less as determined by SPR. In some embodiments, the aC1s-binding proteins bind human aC1s with a K_{D} of 1-10 nM (e.g., 1-5 nM) as determined by SPR. In some embodiments, the aC1s-binding proteins herein bind cynomolgus aC1s with a K_{D} of no more than 5, 4.5, 4, 3.5, 3, 2.5, 2, 1.5, 1, 0.9, 0.8, or 0.7 nM as determined by SPR. In certain embodiments, the aC1s-binding proteins bind cynomolgus aC1s with a K_{D} of 3.5 nM or less as determined by SPR. In some embodiments, the aC1s-binding proteins bind cynomolgus aC1s with a K_{D} of 0.1-0.10 nM (e.g., 0.1-0.5 nM) as determined by SPR. The assay for determining the binding K_{D} can be an SPR assay, e.g., performed as described in detail in Example 4 below. aC1s-binding proteins that bind to both human and cynomolgus aC1s may advantageously allow for pre-clinical studies of the proteins in non-human primates (NHP).

In some embodiments, the aC1s-binding proteins herein inhibit complement. In some embodiments, the aC1s-binding proteins inhibit neuronal complement deposition. In certain embodiments, the aC1s-binding proteins herein have an IC₅₀ of no more than 5, 4.5, 4, 3.5, 3, 2.5, 2, 1.5, or 1 μg/mL (e.g., no more than 4.5 μg/mL) in a Wieslab classical complement pathway assay. In some embodiments, the aC1s-binding proteins herein have an IC₅₀ of 0.1-10 μg/mL (e.g., 0.5-10 or 1-5 μg/mL) in a Wieslab classical complement pathway assay. The assay may be, e.g., as performed as described in detail in Example 4 below.

In certain embodiments, the aC1s-binding protein herein has one or more of the following properties:
a) binds to human aC1s with a K_{D} of 1-5 nM as determined by SPR;
b) binds to cynomolgus aC1s with a K_{D} of 0.1-0.5 nM as determined by SPR;
c) inhibits complement in vitro as determined by a Wieslab classical complement pathway assay;
d) has improved potency and/or high-concentration solution behavior as compared to an anti-aC1s antibody comprising VH and VL set forth in SEQ ID NOs: 10 and 21, respectively; or
e) any combination of a)-d).

In some embodiments, the aC1s-binding protein has all of properties a)-d).

The anti-aC1s binding domains herein may form part of a brain-targeted aC1s-binding protein, such as a binding protein comprising a moiety that facilitates transport across the BBB (e.g., one or more cell-penetrating peptides, an Fc domain modified to bind to a CNS target, or a second binding domain that binds to an endothelial cell receptor of the BBB).

In some embodiments, a brain-targeted aC1s-binding protein herein may comprise an anti-aC1s binding domain herein associated with a cell-penetrating peptide. Cell-penetrating peptides are short peptides that can penetrate biological membranes, facilitating delivery of associated cargos. Where cell-penetrating peptides are targeted to the CNS, they can promote transport of a given cargo across the BBB and into the brain. In some embodiments of a brain-targeted aC1s-binding protein herein, an anti-aC1s binding domain herein may be linked to a cell-penetrating peptide that binds to a CNS target (e.g., TfR or another endothelial cell receptor of the BBB, such as those described herein). The cell-penetrating peptide may be, e.g., a peptide described in Kang et al., Drug Delivery (2022) 29(1):2375-85 (incorporated herein by reference in its entirety), such as the T7 peptide. In some embodiments, the anti-aC1s binding domain of the brain-targeted aC1s-binding protein may be or form part of, e.g., a bivalent antibody, a monovalent antibody, Fab, Fab′, F(ab′)2, or scFv.

In some embodiments, a brain-targeted aC1s-binding protein herein may comprise an anti-aC1s binding domain herein associated with a moiety that facilitates receptor-mediated transcytosis (RMT) at the BBB, e.g., an Fc domain or a fragment thereof wherein one or both chains of the Fc domain, preferably one chain, are engineered to bind to an endothelial cell receptor of the BBB (e.g., TfR or another ECR, such as those described herein). In certain embodiments, the Fc domain is derived from a human IgG1 heavy chain constant region, and may comprise KIH mutations and/or mutations to reduce or eliminate effector function (e.g., “LALA” mutations). In particular embodiments, the Fc domain may bind to TfR, and may be, e.g., a BBB transport vehicle (TV) as described in Kariolis et al., Sci Transl Med. (2020) 12(545):eaay1359 or Arguello et al., J Exp Med. (2022) 219(3):e20211057 (incorporated herein by reference in their entirety). In some embodiments, the anti-aC1s binding domain of the brain-targeted aC1s-binding protein may be or form part of, e.g., a bivalent anti-aC1s antibody or an antigen-binding fragment thereof comprising the Fc domain as defined herein.

In some embodiments, a brain-targeted aC1s-binding protein herein may be or comprise a multispecific, in particular a bispecific, binding protein, as described below.

### B. TfR-Binding Proteins

In some embodiments, the binding proteins of the present disclosure are TfR-binding proteins comprising anti-TfR binding domains, such as anti-TfR antibodies or antigen-binding fragments thereof. In particular embodiments, the TfR-binding proteins herein bind to an epitope on the extracellular region of hTfR and do not interfere with the interaction between hTfR and transferrin, its natural ligand. The TfR-binding proteins are superior BBB transporters and have improved transcytosis efficiency. Further, the present TfR-binding proteins cross-react with cynomolgus monkey TfR (cTfR), thus allowing pre-clinical studies of the proteins in non-human primates (NHP).

In some embodiments, the anti-TfR binding domain comprises HCDR1-3 and LCDR1-3 set forth in SEQ ID NOs: 22, 23, 24, 25, 27, and 28, respectively.

In some embodiments, the anti-TfR binding domain herein comprises the HCDR1-3 in a VH comprising SEQ ID NO: 29 or 30 and the LCDR1-3 in a VL comprising any one of SEQ ID NOs: 31-35. The assignment of CDR regions may be in accordance with any method known in the art, such as IMGT®, Kabat, Chothia, Martin, Contact, or AHo definitions, or any combination of any of these definitions (Kabat plus Chothia, for example). Examples of CDR definitions under different methods are shown below for exemplified anti-TfR antibody 531v25.v6:

HCDRs (in SEQ ID NO: 29)

| Definition | HCDR1 | SEQ | HCDR2 | SEQ | HCDR3 | SEQ |
|---|---|---|---|---|---|---|
| IMGT® | GYTFTRYY | 22 | IDPSVSET | 23 | SQIRLPYYYAMDS | 24 |
| Kabat | RYYLN | 68 | MIDPSVSETHYAQKFQG | 69 | IRLPYYYAMDS | 70 |
| Chothia | GYTFTRY | 73 | DPSVSE | 74 | IRLPYYYAMDS | 70 |
| Contact | TRYYLN | 75 | WIGMIDPSVSETH | 76 | SQIRLPYYYAMD | 77 |

LCDRs (in SEQ ID NO: 31)

| Definition | LCDR1 | SEQ | LCDR2 | SEQ | LCDR3 | SEQ |
|---|---|---|---|---|---|---|
| IMGT® | QDIESF | 25 | YTS | 27 | QQGNTLPRT | 28 |
| Kabat | RASQDIESFLN | 71 | YTSRLQS | 72 | QQGNTLPRT | 28 |
| Chothia | RASQDIESFLN | 71 | YTSRLQS | 72 | QQGNTLPRT | 28 |
| Contact | ESFLNWY | 78 | LLIYYTSRLQ | 79 | QQGNTLPR | 80 |

SEQ: SEQ ID NO:

Thus, for example, the HCDR1-3 sequences of SEQ ID NOs: 22, 23, and 24, respectively, may be replaced in any embodiment described herein by
SEQ ID NOs: 68, 69, and 70, respectively;
SEQ ID NOs: 73, 74, and 70, respectively; or
SEQ ID NOs: 75, 76, and 77, respectively.
   Similarly, the LCDR1-3 sequences of SEQ ID NOs: 25, 27, and 28, respectively, may be replaced in any embodiment described herein by
SEQ ID NOs: 71, 72, and 28, respectively; or
SEQ ID NOs: 78, 79, and 80, respectively.
   Also contemplated is a set of CDRs specified according to any of the methods for defining the CDRs as shown above (e.g., HCDR1 may be defined by the Kabat method, HCDR2 may be defined by the IMGT® method, etc.). These methods or combinations of methods may be used to define the CDRs in the VH or VL of any binding domain herein.

In some embodiments, the anti-TfR binding domain comprises a VH set forth in SEQ ID NO: 30 and a VL set forth in SEQ ID NO: 32 or 34, in any combination.

In some embodiments, the anti-TfR binding domain comprises a VH and a VL with charge mutations to facilitate correct VH/VL pairing, such as in a multispecific context. In anti-TfR VH and VL sequences herein, such charge mutations may comprise, e.g., VH and VL pairs wherein the VH comprises a Q39K mutation and the VL comprises a Q38E mutation. In certain embodiments, the anti-TfR binding domain comprises a VH set forth in SEQ ID NO: 29 and a VL set forth in SEQ ID NO: 31 or 33, in any combination.

In certain embodiments, the anti-TfR binding domain comprises a VH and a VL set forth in SEQ ID NOs: 29 and 31, respectively.

In certain embodiments, the anti-TfR binding domain comprises a VH and a VL set forth in SEQ ID NOs: 29 and 33, respectively.

In certain embodiments, the anti-TfR binding domain comprises a VH and a VL set forth in SEQ ID NOs: 30 and 32, respectively.

In certain embodiments, the anti-TfR binding domain comprises a VH and a VL set forth in SEQ ID NOs: 30 and 34, respectively.

The present disclosure provides an anti-TfR binding protein comprising an anti-TfR binding domain herein. In some embodiments, the TfR-binding protein is an anti-TfR antibody or an antigen-binding fragment thereof. The anti-TfR antibody may comprise any heavy and light chain constant regions described herein. In some embodiments, the anti-TfR antibody comprises a human IgG4 heavy chain constant region, optionally with mutations as described herein. For example, the human IgG4 heavy chain constant region may comprise any one of SEQ ID NOs: 40, 43, and 44 (optionally without the C-terminal lysine, if present). In some embodiments, the antibody comprises a first heavy chain constant region comprising SEQ ID NO: 43 and a second heavy chain constant region comprising SEQ ID NO: 44. In some embodiments, the anti-TfR antibody comprises a human IgG1 heavy chain constant region, optionally with mutations as described herein. For example, the human IgG1 heavy chain constant region may comprise any one of SEQ ID NOs: 37-39, 41, and 42 (optionally without the C-terminal lysine, if present). In some embodiments, the antibody comprises a first heavy chain constant region comprising SEQ ID NO: 41 and a second heavy chain constant region comprising SEQ ID NO: 42. In some embodiments, the anti-TfR antibody comprises a human kappa or lambda light chain constant region. In certain embodiments, the anti-TfR antibody comprises a human kappa light chain constant region, e.g., comprising SEQ ID NO: 45 or 46. In particular embodiments, the anti-TfR antibody comprises a human IgG4 heavy chain constant region comprising SEQ ID NO: 40 or a human IgG1 heavy chain constant region comprising any one of SEQ ID NOs: 37-39, and a human kappa light chain constant region comprising SEQ ID NO: 45. In particular embodiments, the anti-TfR antibody comprises a human IgG4 heavy chain constant region comprising SEQ ID NO: 42 or a human IgG1 heavy chain constant region comprising SEQ ID NO: 44, and a human kappa light chain constant region comprising SEQ ID NO: 46.

In some embodiments, an anti-TfR antibody herein may comprise
an HC comprising SEQ ID NO: 29 and any one of SEQ ID NOs: 37-40 and an LC comprising SEQ ID NO: 31 and SEQ ID NO: 45;
an HC comprising SEQ ID NO: 29 and any one of SEQ ID NOs: 37-40 and an LC comprising SEQ ID NO: 33 and SEQ ID NO: 45;
an HC comprising SEQ ID NO: 30 and any one of SEQ ID NOs: 37-40 and an LC comprising SEQ ID NO: 32 and SEQ ID NO: 45; or
an HC comprising SEQ ID NO: 30 and any one of SEQ ID NOs: 37-40 and an LC comprising SEQ ID NO: 34 and SEQ ID NO: 45.
   Also contemplated is any of the above HC and LC pairs wherein the HC is without the C-terminal lysine, if present. In any of the HC and LC pairs, the HC may further comprise “LS” mutations. Additionally or alternatively, in some embodiments, one HC may comprise “RF” mutations. Additionally or alternatively, in some embodiments, SEQ ID NO: 40 in the HC may be modified by S228P and/or L235E mutations.

The TfR-binding proteins of the present disclosure, including monospecific and bispecific anti-TfR antibodies, bind specifically to TfR with high affinity. In certain embodiments, the TfR-binding protein herein binds human TfR with a K_{D} of no more than 100 nM, 75 nM, 50 nM, 40 nM, 35 nM, 30 nM, 25 nM, 20 nM, 15 nM, or 10 nM as determined by SPR. In certain embodiments, the TfR-binding protein binds human TfR with a K_{D} of no more than 40 nM. In some embodiments, the TfR-binding protein binds human TfR with a K_{D} of 1-50 nM (e.g., 5-25 nM). In certain embodiments, the TfR-binding protein binds cynomolgus TfR with a K_{D} of no more than 100 nM, 95 nM, 90 nM, 85 nM, 80 nM, 75 nM, 70 nM, 65 nM, 60 nM, 55 nM, 50 nM, 45 nM, or 40 nM as determined by SPR. In certain embodiments, the TfR-binding protein binds cynomolgus TfR with a K_{D} of no more than 75 nM. In some embodiments, the TfR-binding protein binds cynomolgus TfR with a K_{D} of 30-90 nM (e.g., 30-80 nM). TfR-binding proteins that bind to both human and cynomolgus TfR may advantageously allow for pre-clinical studies of the proteins in non-human primates (NHP).

In certain embodiments, the TfR-binding proteins herein bind to TfR with a greater affinity than an anti-TfR antibody comprising VH and VL set forth in SEQ ID NOs: 30 and 36, respectively. In particular embodiments, the TfR-binding proteins herein bind to TfR with an affinity at least 1.5, 1.7, 1.9, 2, 2.2, 2.5, or 3 times higher than an anti-TfR antibody comprising VH and VL set forth in SEQ ID NOs: 30 and 36, respectively. The assay for determining the K_{D} can be an SPR assay, e.g., performed as described in detail in Example 3 below.

In particular embodiments, the TfR-binding protein herein has one or more of the following properties:
a) binds to human TfR with a K_{D} of 5-25 nM as determined by SPR;
b) binds to cynomolgus TfR with a K_{D} of 30-80 nM as determined by SPR; or
c) a) and b).

In particular embodiments, the TfR-binding protein has both properties a) and b) (e.g., 531v25.v6 and 531v23).

In some embodiments, the TfR-binding proteins herein provide enhanced brain exposure of a linked cargo (e.g., an anti-aC1s binding domain, such as in a bispecific anti-aC1s/anti-TfR binding protein herein) compared to the unlinked cargo.

It is contemplated that the TfR-binding proteins herein may act as shuttles that cross the blood-brain barrier (“BBB transporters”), thereby facilitating therapeutic entry of a linked cargo into the brain. For example, where the TfR-binding protein forms one antigen-binding portion of a bispecific antibody, it may shuttle the other antigen-binding portion across the BBB. It is to be understood that the linkage of a cargo to a TfR-binding protein herein does not affect, or does not eliminate, its TfR-binding properties.

In some embodiments, the TfR-binding proteins herein, acting as BBB transporters, are linked to a binding domain targeting a protein of the complement system. Proteins of the complement system may include, e.g., those mentioned as targets in Mastellos et al., Nature Reviews Drug Discovery (2019) 18:707-29 (incorporated by reference herein in its entirety). Binding domains targeting proteins of the complement system may be, e.g., from antibodies such as eculizumab, ravulizumab, etc. In certain embodiments, the protein of the complement system may be C1s; here, the binding domain may be from an anti-C1s antibody (e.g., sutimlimab). In particular embodiments, the C1s is in activated form (aC1s), and the binding domain may be, e.g., an anti-aC1s binding domain herein, or an anti-aC1s binding domain as described in, e.g., PCT Patent Publication WO 2022/246154 or WO 2024/112734 (both incorporated by reference herein in their entirety).

In some embodiments, the TfR-binding proteins herein may be multispecific (e.g., bispecific) binding proteins that comprise a binding domain specific for another, distinct target protein. The other target protein may be, e.g., a protein of the complement system, as discussed above. In particular embodiments, the target protein may be C1s (e.g., aC1s).

### II. Multispecific Binding Proteins

The present disclosure also provides aC1s-binding proteins and TfR-binding proteins that are multispecific, e.g., bispecific. In some embodiments, the present disclosure provides a multispecific binding protein (e.g., a bispecific antibody) comprising 1) a domain that binds to aC1s, and 2) a domain that binds to a central nervous system (CNS) target, such as a brain receptor. In certain embodiments, the brain receptor is an endothelial cell receptor of the blood-brain barrier (BBB), for example, a transferrin receptor, insulin receptor, insulin-like growth factor receptor (e.g., IGF1R), low-density lipoprotein receptor, folate receptor, etc. The domain of the multispecific binding protein that binds to the ECR of the BBB may act as a shuttle to transport the anti-aC1s binding domain across the BBB. In particular embodiments, the ECR is transferrin receptor 1 (TfR).

In some embodiments, the anti-aC1s binding domain of the multispecific binding protein competes for binding with, or binds to the same epitope as, an anti-aC1s binding domain described herein.

In some embodiments, the domain of the multispecific binding protein (e.g., bispecific antibody) that binds to the CNS target (“anti-CNS target domain”) binds to an ECR (“anti-ECR binding domain”) such as TfR (“anti-TfR binding domain”). In some embodiments, the anti-TfR binding domain may bind to human TfR with a K_{D} of 10 nM to 1 μM (e.g., 5-50 nM, 5-20 nM, or 20-50 nM) as determined by SPR. In some embodiments, the anti-TfR binding domain of the multispecific binding protein competes for binding with, or binds to the same epitope as, an anti-TfR binding domain described herein.

In some embodiments, the anti-aC1s binding domain of the multispecific binding protein (e.g., bispecific antibody) may be, e.g., an anti-aC1s binding domain as described herein, or an anti-aC1s binding domain as described in PCT Patent Publication WO 2022/246154 or WO 2024/112734. In some embodiments, the anti-TfR target binding domain of the multispecific binding protein may be, e.g., an anti-TfR binding domain as described herein. Any combination of anti-aC1s and anti-TfR binding domains (e.g., the anti-aC1s and anti-TfR binding domains herein) is contemplated.

Sequence identifiers (SEQ ID NOs:) for exemplary anti-aC1s and anti-TfR binding domains, which may be used in the multispecific binding proteins (e.g., bispecific antibodies) herein, are shown in the table below:

| | | | | | | | | | |
|---|---|---|---|---|---|---|---|---|---|
| HCDR1 | HCDR2 | HCDR3 | LCDR1 | LCDR2 | LCDR3 | VH* | VH | VL* | VL |

Anti-aC1s Binding Domain Sequences

| | | | | | | | | | | |
|---|---|---|---|---|---|---|---|---|---|---|
| aC1s-D32E | 1 | 2 | 3 | 4 | 7 | 8 | 9 | 10 | 13 | 14 |
| aC1s-D32Y | 1 | 2 | 3 | 5 | 7 | 8 | 9 | 10 | 15 | 16 |
| aC1s-h088 | 1 | 2 | 3 | 6 | 7 | 8 | 9 | 10 | 19 | — |
| aC1s-088 | 1 | 2 | 3 | 6 | 7 | 8 | 9 | 10 | 20 | 21 |
| VH H5 | 1 | 2 | 3 | — | — | — | 11 | 12 | — | — |
| VL L2 | — | — | — | 4 | 7 | 8 | — | — | 17 | 18 |
| aC1s-DAb1 | 81 | 82 | 83 | 84 | 85 | 86 | — | 100 | — | 101 |
| aC1s-DAb2 | 102 | 103 | 104 | 105 | 106 | 107 | — | 121 | — | 122 |
| aC1s-DAb3 | 123 | 124 | 125 | 126 | 127 | 128 | — | 142 | — | 143 |
| aC1s-DAb4 | 102 | 103 | 144 | 105 | 106 | 147 | — | 149 | — | 150 |
| aC1s-DAb5 | 102 | 103 | 144 | 105 | 106 | 147 | — | 151 | — | 150 |

Anti-TfR Binding Domain Sequences

| | | | | | | | | | | |
|---|---|---|---|---|---|---|---|---|---|---|
| 531v25.v6 | 22 | 23 | 24 | 25 | 27 | 28 | 29 | 30 | 31 | 32 |
| 531v23 | 22 | 23 | 24 | 26 | 27 | 28 | 29 | 30 | 33 | 34 |
| 531v25 | 22 | 23 | 24 | 26 | 27 | 28 | 29 | 30 | 35 | 36 |

In some embodiments, the anti-aC1s binding domain comprises HCDR1-3 and LCDR1-3 of an anti-C1s binding domain as described herein. In certain embodiments, the H-CDR1-3 and LCDR1-3 are set forth in
SEQ ID NOs: 1, 2, 3, 4, 7, and 8, respectively;
SEQ ID NOs: 1, 2, 3, 5, 7, and 8, respectively; or
SEQ ID NOs: 1, 2, 3, 6, 7, and 8, respectively.
   In certain embodiments, the H-CDR1-3 and LCDR1-3 are set forth in
SEQ ID NOs: 81, 82, 83, 84, 85, and 86, respectively;
SEQ ID NOs: 102, 103, 104, 105, 106, and 107, respectively;
SEQ ID NOs: 123, 124, 125, 126, 127, and 128, respectively;
SEQ ID NOs: 102, 103, 144, 105, 106, and 147, respectively; or
SEQ ID NOs: 102, 103, 144, 105, 106, and 148, respectively.

As discussed above in reference to exemplified anti-aC1s antibody aC1s-D32E, different CDR definitions also may be used for anti-aC1s binding domains from any of the other anti-aC1s antibodies herein. For example, the HCDR1-3 sequences of SEQ ID NOs: 81-83, respectively, may be replaced in any embodiment described herein by
SEQ ID NOs: 87, 88, and 89, respectively;
SEQ ID NOs: 92, 93, and 89, respectively; or
SEQ ID NOs: 94, 95, and 96, respectively; and/or
the LCDR1-3 sequences of SEQ ID NOs: 84-86, respectively, may be replaced in any embodiment described herein by
SEQ ID NOs: 90, 91, and 86, respectively; or
SEQ ID NOs: 97, 98, and 99, respectively.

The HCDR1-3 sequences of SEQ ID NOs: 102, 103, and 104, respectively, may be replaced in any embodiment described herein by
SEQ ID NOs: 108, 109, and 110, respectively;
SEQ ID NOs: 113, 114, and 110, respectively; or
SEQ ID NOs: 115, 116, and 117, respectively; and/or
the LCDR1-3 sequences of SEQ ID NOs: 105, 106, and 107, respectively, may be replaced in any embodiment described herein by
SEQ ID NOs: 111, 112, and 107, respectively; or
SEQ ID NOs: 118, 119, and 120, respectively.

The HCDR1-3 sequences of SEQ ID NOs: 123-125, respectively, may be replaced in any embodiment described herein by
SEQ ID NOs: 129, 130, and 131, respectively;
SEQ ID NOs: 134, 135, and 131, respectively; or
SEQ ID NOs: 136, 137, and 138, respectively; and/or
the LCDR1-3 sequences of SEQ ID NOs: 126, 127, and 128, respectively, may be replaced in any embodiment described herein by
SEQ ID NOs: 132, 133, and 128, respectively; or
SEQ ID NOs: 139, 140, and 141, respectively.

The HCDR1-3 sequences of SEQ ID NOs: 102, 103, and 144, respectively, may be replaced in any embodiment described herein by
SEQ ID NOs: 108, 109, and 145, respectively;
SEQ ID NOs: 113, 114, and 145, respectively; or
SEQ ID NOs: 115, 116, and 146, respectively; and/or
the LCDR1-3 sequences of SEQ ID NOs: 105, 106, and 147, respectively, may be replaced in any embodiment described herein by
SEQ ID NOs: 111, 112, and 147, respectively; or
SEQ ID NOs: 118, 119, and 148, respectively.

In some embodiments, the anti-aC1s binding domain comprises a VH at least 80% (e.g., at least 80, 85, 90, 91, 92, 93, 94, 95, 96, 97, 98, or 99%) identical to any one of SEQ ID NOs: 9-12 and a VL at least 80% (e.g., at least 80, 85, 90, 91, 92, 93, 94, 95, 96, 97, 98, or 99%) identical to any one of SEQ ID NOs: 13-21, in any combination. For example, the anti-aC1s binding domain may comprise VH and a VL at least 80% (e.g., at least 80, 85, 90, 91, 92, 93, 94, 95, 96, 97, 98, or 99%) identical to:
SEQ ID NOs: 9 and 13, respectively;
SEQ ID NOs: 9 and 15, respectively;
SEQ ID NOs: 9 and 17, respectively;
SEQ ID NOs: 9 and 19, respectively;
SEQ ID NOs: 9 and 20, respectively;
SEQ ID NOs: 10 and 14, respectively;
SEQ ID NOs: 10 and 16, respectively;
SEQ ID NOs: 10 and 18, respectively;
SEQ ID NOs: 10 and 21, respectively;
SEQ ID NOs: 11 and 13, respectively;
SEQ ID NOs: 11 and 15, respectively;
SEQ ID NOs: 11 and 17, respectively;
SEQ ID NOs: 11 and 19, respectively;
SEQ ID NOs: 11 and 20, respectively;
SEQ ID NOs: 12 and 14, respectively;
SEQ ID NOs: 12 and 16, respectively;
SEQ ID NOs: 12 and 18, respectively; or
SEQ ID NOs: 12 and 21, respectively.

In some embodiments, the anti-aC1s binding domain comprises a VH selected from SEQ ID NOs: 9-12 and a VL selected from any one of SEQ ID NOs: 13-21, in any combination.

In some embodiments, the anti-aC1s binding domain comprises VH and VL set forth in:
SEQ ID NOs: 9 and 13, respectively;
SEQ ID NOs: 9 and 15, respectively;
SEQ ID NOs: 9 and 17, respectively;
SEQ ID NOs: 9 and 19, respectively;
SEQ ID NOs: 9 and 20, respectively;
SEQ ID NOs: 10 and 14, respectively;
SEQ ID NOs: 10 and 16, respectively;
SEQ ID NOs: 10 and 18, respectively;
SEQ ID NOs: 10 and 21, respectively;
SEQ ID NOs: 11 and 13, respectively;
SEQ ID NOs: 11 and 15, respectively;
SEQ ID NOs: 11 and 17, respectively;
SEQ ID NOs: 11 and 19, respectively;
SEQ ID NOs: 11 and 20, respectively;
SEQ ID NOs: 12 and 14, respectively;
SEQ ID NOs: 12 and 16, respectively;
SEQ ID NOs: 12 and 18, respectively; or
SEQ ID NOs: 12 and 21, respectively.

In some embodiments, the anti-aC1s binding domain may comprise VH and a VL at least 80% (e.g., at least 80, 85, 90, 91, 92, 93, 94, 95, 96, 97, 98, or 99%) identical to:
SEQ ID NOs: 100 and 101, respectively;
SEQ ID NOs: 121 and 122, respectively;
SEQ ID NOs: 142 and 143, respectively;
SEQ ID NOs: 149 and 150, respectively; or
SEQ ID NOs: 151 and 150, respectively.

In some embodiments, the anti-aC1s binding domain comprises VH and VL set forth in:
SEQ ID NOs: 100 and 101, respectively;
SEQ ID NOs: 121 and 122, respectively;
SEQ ID NOs: 142 and 143, respectively;
SEQ ID NOs: 149 and 150, respectively; or
SEQ ID NOs: 151 and 150, respectively.

In certain embodiments, the anti-TfR binding domain of the multispecific binding protein herein binds human TfR with a K_{D} of no more than 1 μM, 900 nM, 800 nM, 700 nM, 600 nM, 500 nM, 400 nM, 300 nM, 200 nM, 100 nM, 75 nM, 50 nM, 40 nM, 35 nM, 30 nM, 25 nM, 20 nM, 15 nM, or 10 nM as determined by SPR. In certain embodiments, the anti-TfR binding domain binds human TfR with a K_{D} of no more than 40 nM. In some embodiments, the anti-TfR binding domain binds human TfR with a K_{D} of 10 nM to 1 μM. In some embodiments, the anti-TfR binding domain binds human TfR with a K_{D} of 100 nM to 200 nM. In some embodiments, the anti-TfR binding domain binds human TfR with a K_{D} of 200 nM to 400 nM. In some embodiments, the anti-TfR binding domain binds human TfR with a K_{D} of 500 nM to 700 nM. In some embodiments, the anti-TfR binding domain binds human TfR with a K_{D} of 10-50 nM (e.g., 25-45 nM). In certain embodiments, the anti-TfR binding domain binds cynomolgus TfR with a K_{D} of no more than 1 μM, 900 nM, 800 nM, 700 nM, 600 nM, 500 nM, 400 nM, 300 nM, 200 nM, 100 nM, 95 nM, 90 nM, 85 nM, 80 nM, 75 nM, 70 nM, 65 nM, 60 nM, 55 nM, 50 nM, 45 nM, or 40 nM as determined by SPR. In certain embodiments, the anti-TfR binding domain binds cynomolgus TfR with a K_{D} of no more than 75 nM. In some embodiments, the anti-TfR binding domain binds cynomolgus TfR with a K_{D} of 10 nM to 1 μM. In some embodiments, the anti-TfR binding domain binds cynomolgus TfR with a K_{D} of 100 nM to 200 nM. In some embodiments, the anti-TfR binding domain binds cynomolgus TfR with a K_{D} of 200 nM to 400 nM. In some embodiments, the anti-TfR binding domain binds cynomolgus TfR with a K_{D} of 500 nM to 700 nM. In some embodiments, the anti-TfR binding domain binds cynomolgus TfR with a K_{D} of 30-90 nM (e.g., 30-80 nM).

In some embodiments, the anti-TfR binding domain of the multispecific binding protein herein is the binding domain of an anti-TfR antibody or an antigen-binding fragment thereof disclosed in PCT Patent Publication WO 2024/121755. In some embodiments, the anti-TfR binding domain competes for binding with, or binds to the same epitope as, an anti-TfR antibody described herein.

In some embodiments, the anti-TfR binding domain comprises HCDR1-3 and LCDR1-3 set forth in
SEQ ID NOs: 22, 23, 24, 25, 27, and 28, respectively; or
SEQ ID NOs: 22, 23, 24, 26, 27, and 28, respectively.

In some embodiments, the anti-TfR binding domain comprises a VH at least 80% (e.g., at least 80, 85, 90, 91, 92, 93, 94, 95, 96, 97, 98, or 99%) identical to any one of SEQ ID NO: 29 or 30 and a VL at least 80% (e.g., at least 80, 85, 90, 91, 92, 93, 94, 95, 96, 97, 98, or 99%) identical to any one of SEQ ID NOs: 31-36, in any combination. For example, the anti-TfR binding domain may comprise a VH and a VL at least 80% (e.g., at least 80, 85, 90, 91, 92, 93, 94, 95, 96, 97, 98, or 99%) identical to:
SEQ ID NOs: 29 and 31, respectively;
SEQ ID NOs: 29 and 33, respectively;
SEQ ID NOs: 29 and 35, respectively;
SEQ ID NOs: 30 and 32, respectively;
SEQ ID NOs: 30 and 34, respectively; or
SEQ ID NOs: 30 and 36, respectively.

In some embodiments, the anti-TfR binding domain comprises a VH of SEQ ID NO: 29 or 30 and a VL selected from SEQ ID NOs: 31-36, in any combination.

In some embodiments, the anti-TfR binding domain comprises a VH and a VL set forth in
SEQ ID NOs: 29 and 31, respectively;
SEQ ID NOs: 29 and 33, respectively;
SEQ ID NOs: 29 and 35, respectively;
SEQ ID NOs: 30 and 32, respectively;
SEQ ID NOs: 30 and 34, respectively; or
SEQ ID NOs: 30 and 36, respectively.

Any combination of an anti-aC1s and an anti-TfR binding domain described herein is contemplated for the multispecific binding proteins (e.g., bispecific antibodies) herein.

In some embodiments, a multispecific binding protein herein may comprise
an anti-aC1s binding domain comprising HCDR1-3 and LCDR1-3 set forth in SEQ ID NOs: 1, 2, 3, 4, 7, and 8, respectively, and an anti-TfR binding domain comprising HCDR1-3 and LCDR1-3 set forth in SEQ ID NOs: 22, 23, 24, 25, 27, and 28, respectively;
an anti-aC1s binding domain comprising HCDR1-3 and LCDR1-3 set forth in SEQ ID NOs: 1, 2, 3, 4, 7, and 8, respectively, and an anti-TfR binding domain comprising HCDR1-3 and LCDR1-3 set forth in SEQ ID NOs: 22, 23, 24, 26, 27, and 28, respectively;
an anti-aC1s binding domain comprising HCDR1-3 and LCDR1-3 set forth in SEQ ID NOs: 1, 2, 3, 5, 7, and 8, respectively, and an anti-TfR binding domain comprising HCDR1-3 and LCDR1-3 set forth in SEQ ID NOs: 22, 23, 24, 25, 27, and 28, respectively;
an anti-aC1s binding domain comprising HCDR1-3 and LCDR1-3 set forth in SEQ ID NOs: 1, 2, 3, 5, 7, and 8, respectively, and an anti-TfR binding domain comprising HCDR1-3 and LCDR1-3 set forth in SEQ ID NOs: 22, 23, 24, 26, 27, and 28, respectively;
an anti-aC1s binding domain comprising HCDR1-3 and LCDR1-3 set forth in SEQ ID NOs: 1, 2, 3, 6, 7, and 8, respectively, and an anti-TfR binding domain comprising HCDR1-3 and LCDR1-3 set forth in SEQ ID NOs: 22, 23, 24, 25, 27, and 28, respectively;
an anti-aC1s binding domain comprising HCDR1-3 and LCDR1-3 set forth in SEQ ID NOs: 1, 2, 3, 6, 7, and 8, respectively, and an anti-TfR binding domain comprising HCDR1-3 and LCDR1-3 set forth in SEQ ID NOs: 22, 23, 24, 26, 27, and 28, respectively
an anti-aC1s binding domain comprising HCDR1-3 and LCDR1-3 set forth in SEQ ID NOs: 81, 82, 83, 84, 85, and 86, respectively, and an anti-TfR binding domain comprising HCDR1-3 and LCDR1-3 set forth in SEQ ID NOs: 22, 23, 24, 25, 27, and 28, respectively;
an anti-aC1s binding domain comprising HCDR1-3 and LCDR1-3 set forth in SEQ ID NOs: 81, 82, 83, 84, 85, and 86, respectively, and an anti-TfR binding domain comprising HCDR1-3 and LCDR1-3 set forth in SEQ ID NOs: 22, 23, 24, 26, 27, and 28, respectively;
an anti-aC1s binding domain comprising HCDR1-3 and LCDR1-3 set forth in SEQ ID NOs: 102, 103, 104, 105, 106, and 107, respectively, and an anti-TfR binding domain comprising HCDR1-3 and LCDR1-3 set forth in SEQ ID NOs: 22, 23, 24, 25, 27, and 28, respectively;
an anti-aC1s binding domain comprising HCDR1-3 and LCDR1-3 set forth in SEQ ID NOs: 102, 103, 104, 105, 106, and 107, respectively, and an anti-TfR binding domain comprising HCDR1-3 and LCDR1-3 set forth in SEQ ID NOs: 22, 23, 24, 26, 27, and 28, respectively;
an anti-aC1s binding domain comprising HCDR1-3 and LCDR1-3 set forth in SEQ ID NOs: 123, 124, 125, 126, 127, and 128, respectively, and an anti-TfR binding domain comprising HCDR1-3 and LCDR1-3 set forth in SEQ ID NOs: 22, 23, 24, 25, 27, and 28, respectively;
an anti-aC1s binding domain comprising HCDR1-3 and LCDR1-3 set forth in SEQ ID NOs: 123, 124, 125, 126, 127, and 128, respectively, and an anti-TfR binding domain comprising HCDR1-3 and LCDR1-3 set forth in SEQ ID NOs: 22, 23, 24, 26, 27, and 28, respectively;
an anti-aC1s binding domain comprising HCDR1-3 and LCDR1-3 set forth in SEQ ID NOs: 102, 103, 144, 105, 106, and 147, respectively, and an anti-TfR binding domain comprising HCDR1-3 and LCDR1-3 set forth in SEQ ID NOs: 22, 23, 24, 25, 27, and 28, respectively;
an anti-aC1s binding domain comprising HCDR1-3 and LCDR1-3 set forth in SEQ ID NOs: 102, 103, 144, 105, 106, and 147, respectively, and an anti-TfR binding domain comprising HCDR1-3 and LCDR1-3 set forth in SEQ ID NOs: 22, 23, 24, 26, 27, and 28, respectively;
an anti-aC1s binding domain comprising HCDR1-3 and LCDR1-3 set forth in SEQ ID NOs: 102, 103, 144, 105, 106, and 148, respectively, and an anti-TfR binding domain comprising HCDR1-3 and LCDR1-3 set forth in SEQ ID NOs: 22, 23, 24, 25, 27, and 28, respectively; or
an anti-aC1s binding domain comprising HCDR1-3 and LCDR1-3 set forth in SEQ ID NOs: 102, 103, 144, 105, 106, and 148, respectively, and an anti-TfR binding domain comprising HCDR1-3 and LCDR1-3 set forth in SEQ ID NOs: 22, 23, 24, 26, 27, and 28, respectively.

In some embodiments, a multispecific binding protein herein may comprise
an anti-aC1s binding domain comprising VH and VL set forth in SEQ ID NOs: 9 and 13, respectively, and an anti-TfR binding domain comprising VH and VL set forth in SEQ ID NOs: 29 and 31, respectively;
an anti-aC1s binding domain comprising VH and VL set forth in SEQ ID NOs: 11 and 13, respectively, and an anti-TfR binding domain comprising VH and VL set forth in SEQ ID NOs: 29 and 31, respectively;
an anti-aC1s binding domain comprising VH and VL set forth in SEQ ID NOs: 9 and 17, respectively, and an anti-TfR binding domain comprising VH and VL set forth in SEQ ID NOs: 29 and 31, respectively;
an anti-aC1s binding domain comprising VH and VL set forth in SEQ ID NOs: 11 and 17, respectively, and an anti-TfR binding domain comprising VH and VL set forth in SEQ ID NOs: 29 and 31, respectively;
an anti-aC1s binding domain comprising VH and VL set forth in SEQ ID NOs: 100 and 101, respectively, and an anti-TfR binding domain comprising VH and VL set forth in SEQ ID NOs: 29 and 31, respectively;
an anti-aC1s binding domain comprising VH and VL set forth in SEQ ID NOs: 121 and 122, respectively, and an anti-TfR binding domain comprising VH and VL set forth in SEQ ID NOs: 29 and 31, respectively;
an anti-aC1s binding domain comprising VH and VL set forth in SEQ ID NOs: 142 and 143, respectively, and an anti-TfR binding domain comprising VH and VL set forth in SEQ ID NOs: 29 and 31, respectively;
an anti-aC1s binding domain comprising VH and VL set forth in SEQ ID NOs: 149 and 150, respectively, and an anti-TfR binding domain comprising VH and VL set forth in SEQ ID NOs: 29 and 31, respectively; or
an anti-aC1s binding domain comprising VH and VL set forth in SEQ ID NOs: 151 and 150, respectively, and an anti-TfR binding domain comprising VH and VL set forth in SEQ ID NOs: 29 and 31, respectively.
   The VH and VL of any anti-aC1s binding domain herein may be combined with the VH and VL of any anti-TfR binding domain herein in a multispecific binding protein herein.

In some embodiments, a multispecific binding protein (e.g., bispecific antibody) herein may be monovalent for aC1s and monovalent for TfR. In some embodiments, the multispecific binding protein may have one arm comprising the anti-aC1s binding domain and another arm comprising the anti-CNS target binding domain (e.g., anti-TfR binding domain). In certain embodiments, the multispecific binding protein may be a bispecific binding protein (e.g., a bispecific antibody) comprising two heavy chains and two light chains, wherein the anti-aC1s binding domain is formed by one pair of heavy and light chains, and the anti-CNS target binding domain is formed by the other pair of heavy and light chains.

The anti-aC1s binding domain of the multispecific binding protein (e.g., bispecific antibody) herein may be functionally linked, e.g., by noncovalent association, chemical coupling, protein fusion, etc., to the anti-CNS target binding domain (e.g., wherein the CNS target is an endothelial cell receptor such as TfR). In some embodiments, the multispecific binding protein is a multispecific antibody (e.g., a bispecific antibody). In certain embodiments, the anti-aC1s binding domain comprises a Fab with a VH and a VL (e.g., of an anti-aC1s binding domain described herein), wherein the Fab is linked to a first Fc chain, and the anti-CNS target binding domain comprises a Fab with a VH and a VL (e.g., of an anti-TfR binding domain described herein), wherein the Fab is linked to a second Fc chain, wherein the two Fc chains pair to form an Fc region.

Where the multispecific binding protein comprises an Fc region and/or is a multispecific antibody (e.g., a bispecific antibody), it may be of any immunoglobulin isotype, such as human IgG (e.g., IgG1, IgG2, IgG3, or IgG4). For example, a multispecific antibody herein may comprise a human IgG1 or IgG4 heavy chain constant region, e.g., with mutations to improve the clinical potential of the antibody (such as mutations that reduce or eliminate effector functions, improve the serum half-life of the antibody, or improve manufacturing and yield of the antibody, as described herein, in any combination).

In embodiments where the multispecific antibody has two different heavy chains (e.g., one comprising the VH of the anti-aC1s binding domain and one comprising the VH of the anti-CNS target binding domain), to promote heterodimerization of the two different heavy chains during manufacturing, mutations may be introduced to the heavy chains to physically (e.g., by steric hinderance, “knobs” into “holes”) or biochemically (e.g., by electrostatic interactions) deter coupling of heavy chains of the same type. For example, knobs-in-holes (KIH) mutations can be introduced to create a “knob” heavy chain and a “hole” heavy chain that preferentially pair with each other. Exemplary KIH mutations comprise S354C and T366W in one heavy chain and Y349C/T366S/L368A/Y407V in the other heavy chain. See also PCT Patent Publication WO 2009/089004 and U.S. Pat. No. 8,642,745; and Brinkmann and Kontermann, MAbs. (2017) 9(2):182-212, hereby incorporated by reference herein in their entirety.

In some embodiments, a multispecific antibody herein comprises an IgG heavy chain constant region, in combination with a light chain constant region, that comprise CR3/NN3 charge-pair mutations that facilitate specific heavy and light chain pairing (CR3: T187E mutation in the heavy chain constant region and N137K/S114A mutations in the light chain constant region; NN3: K213E and K218D mutations in the heavy chain constant region and E123K and D122K mutations in the light chain constant region). The IgG heavy chain constant region may further comprise any mutation or combination of mutations described below.

In some embodiments, a multispecific antibody herein is of human isotype subclass IgG4 and comprises two different heavy chain constant regions comprising:
a) KIH mutations, (e.g., S354C and T336W knob mutations and Y349C, T366S, L368A, and Y407V hole mutations),
b) S228P,
c) L235E,
d) M428L and N434S,
e) H435R and Y436F, or
f) any combination of a)-e).
   In certain embodiments, the multispecific antibody comprises a first heavy chain constant region with a) (e.g., knob), b), c) and d) mutations, and a second heavy chain constant region with a) (e.g., hole), b), c), d), and e) mutations. In particular embodiments, for example, the multispecific antibody comprises a knob heavy chain constant region comprising SEQ ID NO: 43 and a hole heavy chain constant region comprising SEQ ID NO: 44.

In some embodiments, a multispecific antibody herein is of human isotype subclass IgG1 and comprises two different heavy chain constant regions comprising:
a) KIH mutations, (e.g., S354C and T336W knob mutations and Y349C, T366S, L368A, and Y407V hole mutations),
b) L234A and L235A,
c) A237G, P329A, A330S, and P331S,
d) M428L and N434S,
e) H435R and Y436F, or
f) any combination of a)-e).
   In certain embodiments, the multispecific antibody comprises a first heavy chain constant region with a) (e.g., knob), b), c), and d) mutations, and a second heavy chain constant region with a) (e.g., hole), b), c), d), and e) mutations. In particular embodiments, for example, the multispecific antibody comprises a knob heavy chain constant region comprising SEQ ID NO: 41 and a hole heavy chain constant region comprising SEQ ID NO: 42.

In some embodiments, a multispecific antibody herein comprises a human kappa or lambda light chain constant region. In certain embodiments, the bispecific antibody comprises a kappa light chain constant region comprising SEQ ID NO: 45 or SEQ ID NO: 46. In particular embodiments, a multispecific antibody comprising a kappa light chain constant region sequence of SEQ ID NO: 46 further comprises a heavy chain constant region sequence of SEQ ID NO: 42 or 44 (pairing facilitated by charge-pair mutations).

In particular embodiments, a multispecific binding protein (e.g., bispecific antibody) herein that binds to aC1s and TfR may comprise
a first heavy chain that comprises SEQ ID NO: 47, a second heavy chain that comprises SEQ ID NO: 51, a first light chain that comprises SEQ ID NO: 49, and a second light chain that comprises SEQ ID NO: 52;
a first heavy chain that comprises SEQ ID NO: 47, a second heavy chain that comprises SEQ ID NO: 51, a first light chain that comprises SEQ ID NO: 50, and a second light chain that comprises SEQ ID NO: 52;
a first heavy chain that comprises SEQ ID NO: 48, a second heavy chain that comprises SEQ ID NO: 51, a first light chain that comprises SEQ ID NO: 49, and a second light chain that comprises SEQ ID NO: 52; or
a first heavy chain that comprises SEQ ID NO: 48, a second heavy chain that comprises SEQ ID NO: 51, a first light chain that comprises SEQ ID NO: 50, and a second light chain that comprises SEQ ID NO: 52.
   The first heavy chain may pair with the first light chain, and the second heavy chain may pair with the second light chain.

In other particular embodiments, a multispecific binding protein (e.g., bispecific antibody) herein that binds to aC1s and TfR may comprise
a first heavy chain that comprises SEQ ID NOs: 100 and 44, a second heavy chain that comprises SEQ ID NO: 51, a first light chain that comprises SEQ ID NOs: 101 and 46, and a second light chain that comprises SEQ ID NO: 52;
a first heavy chain that comprises SEQ ID NOs: 121 and 44, a second heavy chain that comprises SEQ ID NO: 51, a first light chain that comprises SEQ ID NOs: 122 and 46, and a second light chain that comprises SEQ ID NO: 52;
a first heavy chain that comprises SEQ ID NOs: 142 and 44, a second heavy chain that comprises SEQ ID NO: 51, a first light chain that comprises SEQ ID NOs: 143 and 46, and a second light chain that comprises SEQ ID NO: 52;
a first heavy chain that comprises SEQ ID NOs: 149 and 44, a second heavy chain that comprises SEQ ID NO: 51, a first light chain that comprises SEQ ID NOs: 150 and 46, and a second light chain that comprises SEQ ID NO: 52; or
a first heavy chain that comprises SEQ ID NOs: 151 and 44, a second heavy chain that comprises SEQ ID NO: 51, a first light chain that comprises SEQ ID NOs: 150 and 46, and a second light chain that comprises SEQ ID NO: 52.
   The first heavy chain may pair with the first light chain, and the second heavy chain may pair with the second light chain.

In some embodiments, the multispecific binding protein (e.g., bispecific antibody) herein binds to human aC1s with a K_{D} of no more than 5, 4.5, 4, 3.5, 3, 2.5, 2, 1.5, 1, 0.9, 0.8, or 0.7 nM. In certain embodiments, the multispecific binding protein binds human aC1s with a K_{D} of no more than 3.5 nM. In some embodiments, the multispecific binding protein binds cynomolgus aC1s with a K_{D} of no more than 5, 4.5, 4, 3.5, 3, 2.5, or 2 nM. In certain embodiments, the multispecific binding protein binds cynomolgus aC1s with a K_{D} of no more than 3.5 nM. In some embodiments, the multispecific binding protein binds human aC1s with a K_{D} of 1-10 nM (e.g., 1-5 nM). In some embodiments, the multispecific binding protein binds cynomolgus aC1s with a K_{D} of 0.1-1 nM (e.g., 0.1-0.5 nM). The assay for determining the binding K_{D} can be an SPR assay, e.g., performed as described in detail in Example 4 below.

In certain embodiments, the multispecific binding protein binds human TfR with a K_{D} of no more than 60, 50, 40, 35, 30, 25, 20, 15, or 10 nM. In certain embodiments, the multispecific binding protein binds human TfR with a K_{D} of no more than 40 nM. In some embodiments, the multispecific binding protein binds human TfR with a K_{D} of 10-50 nM (e.g., 25-45 nM). In certain embodiments, the multispecific binding protein binds cynomolgus TfR with a K_{D} of no more than 100, 95, 90, 85, 80, 75, 70, 65, 60, 55, 50, 45, or 40 nM. In certain embodiments, the multispecific binding protein binds cynomolgus TfR with a K_{D} of no more than 75 nM. In some embodiments, the multispecific binding protein binds cynomolgus TfR with a K_{D} of 30-90 nM (e.g., 30-80 nM). The assay for determining the binding K_{D} can be an SPR assay, e.g., performed as described in detail in Example 4 below.

In certain embodiments, the multispecific binding protein (e.g., bispecific antibody) herein binds to both human and cynomolgus TfR (e.g., with K_{D}s as described above). In particular embodiments, the multispecific binding protein herein binds to both human and cynomolgus aC1s, and both human and cynomolgus TfR (e.g., with K_{D}s as described above). Such multispecific binding protein may advantageously allow for pre-clinical studies of the proteins in non-human primates (NHP).

In certain embodiments, the multispecific binding protein (e.g., bispecific antibody) herein crosses the BBB and achieves a maximal concentration in brain that is at least 2-fold, 3-fold, 4-fold, 5-fold, 6-fold, 10-fold, 20-fold and up to 30-fold higher than a monospecific aC1s-binding protein comprising the corresponding anti-aC1s binding domain.

In certain embodiments, the multispecific binding protein (e.g., bispecific antibody) herein achieves a maximal concentration in the CSF at least 1.5, 2, 3, 4, 5, 6, 7, 8, 9, 10, 11, 12, 13, 14, 15, 16, 17, 18, 19, 20, 21, 22, 23, 24, 25, 26, 27, 28, 29, or 30-fold higher than a monospecific aC1s-binding protein comprising the corresponding anti-aC1s binding domain.

In certain embodiments, the multispecific binding protein (e.g., bispecific antibody) herein inhibits complement and/or inhibits neuronal complement deposition. In particular embodiments, the bispecific antibody has an IC₅₀ of no more than 3, 2.75, 2.5, 2.25, 2, 1.75, 1.5, 1.25, or 1 μg/mL in a Wieslab classical complement pathway assay. In some embodiments, the bispecific antibody has an IC₅₀ of 0.1-10 μg/mL (e.g., 0.5-3 μg/mL) in a Wieslab classical complement pathway assay. In particular embodiments, the bispecific antibody has an IC₉₀ of no more than 12, 11, 10.5, 10, 9.5, 9, 8.5, 8, 7.5, or 7 μg/mL in a Wieslab classical complement pathway assay. In some embodiments, the bispecific antibody has an IC₉₀ of 1-20 μg/mL (e.g., 5-15 μg/mL) in a Wieslab classical complement pathway assay. The assay may be, e.g., performed as described in detail in Example 4 below.

In some embodiments, the multispecific binding protein (e.g., bispecific antibody) herein has an IC₅₀ of no more than 30, 25, 20, 15, or 10 μg/mL (or an IC₅₀ of 10-30 μg/mL) in a complement inhibition assay measuring inhibition of C3d deposition in iPSC tricultures (neurons, microglia, astrocytes). The assay may be, e.g., performed as described in detail in Example 5 below.

A multispecific binding protein with any combination of the above properties is also contemplated.

In certain embodiments, the multispecific binding protein (e.g., bispecific antibody) herein has one or more of the following properties:
a) binds to human aC1s with a K_{D} of 1-10 nM as determined by SPR;
b) binds to cynomolgus aC1s with a K_{D} of 0.1-1 nM as determined by SPR;
c) binds to human TfR with a K_{D} of 10-50 nM as determined by SPR;
d) binds to cynomolgus TfR with a K_{D} of 30-90 nM as determined by SPR;
e) crosses the blood-brain barrier and achieves a higher maximal concentration in brain than a monospecific aC1s-binding protein comprising the anti-aC1s binding domain;
f) achieves a higher maximal concentration in the CSF than a monospecific aC1s-binding protein comprising the anti-aC1s binding domain;
g) inhibits neuronal complement deposition in vitro in a Wieslab classical complement pathway assay;
h) inhibits C3d deposition in vitro in iPSC tricultures; or
i) any combination of a)-h).

In some embodiments, the multispecific binding protein has all of properties a)-h).

### III. Making of Binding Proteins

The binding proteins (e.g., monospecific or multispecific binding proteins) described herein may be produced recombinantly using isolated nucleic acid molecules such as expression constructs encoding each chain of the proteins. Biomolecules (e.g., nucleic acid or polypeptide molecules) referred to herein as “isolated” or “purified” are those that (1) have been separated away from the biomolecules (e.g., nucleic acids of the genomic DNA or cellular RNA, or polypeptides, of their source of origin; and/or (2) do not occur in nature. The encoding sequences for each polypeptide chain may be cloned into a single vector or cloned into separate vectors.

Methods of producing proteins such as antibodies are well known. The present binding proteins such as antibodies may be produced in, e.g., mammalian host cells, using appropriate expression constructs. Mammalian cell lines available as hosts for expression include many immortalized cell lines available from the American Type Culture Collection (ATCC). These include, inter alia, Chinese hamster ovary (CHO) cells, NS0 cells, SP2 cells, HEK-293T cells, 293 Freestyle cells (Invitrogen), NIH-3T3 cells, HeLa cells, baby hamster kidney (BHK) cells, African green monkey kidney cells (COS), human hepatocellular carcinoma cells (e.g., Hep G2), A549 cells, and a number of other cell lines. Other cell lines that may be used are insect cell lines, such as Sf9 or Sf21 cells, and yeast cell lines. Cell lines may be selected based on their expression levels. The binding proteins may be isolated and purified from the host cell culture using well known methods, such as centrifugation, ultracentrifugation, protein A, protein G, protein A/G, or protein L purification, and/or ion exchange chromatography.

### IV. Pharmaceutical Compositions and Use

The present disclosure also provides pharmaceutical compositions comprising the binding proteins (e.g., monospecific or multispecific binding proteins) herein. The pharmaceutical compositions may comprise one or more pharmaceutically acceptable excipients, carriers, or diluents. As used herein, “pharmaceutically acceptable” with reference to a carrier,” “excipient,” or “diluent” includes appropriate solvents, dispersion media, antibacterial and antifungal agents, isotonic agents, and the like. In some embodiments, the pharmaceutical composition is a sterile aqueous solution, and may comprise a buffer; a surfactant; a polyol; an antioxidant; and/or a chelating agent. In some embodiments, the pharmaceutical composition is provided in a lyophilized form and is reconstituted before administration. In certain embodiments, lyophilized antibody formulations may comprise a bulking agent.

The pharmaceutical composition may be administered to patients by parenteral administration (e.g., by injection or infusion). For example, the pharmaceutical composition may be administered by an intravenous, intracerebral, intracranial, or spinal route.

The pharmaceutical compositions comprising an aC1s-binding protein herein are useful in treating a human patient with, or at risk of developing, a neurological complement-mediated disorder (i.e., a disorder in which the complement cascade is dysregulated or aberrantly activated). When paired with an anti-TfR binding protein in a multispecific or bispecific binding protein (e.g., a bispecific antibody), the aC1s-binding protein may be shuttled across the blood-brain barrier. The pharmaceutical composition comprising a bispecific binding protein herein that binds to aC1s and TfR thus is particularly useful for treating a human patient with, or at risk of developing, a neurological complement-mediated disorder. In some embodiments, the neurological complement-mediated disorder is amyotrophic lateral sclerosis (ALS), Alzheimer's disease, Huntington's disease, an autoimmune peripheral neuropathy, a neurodegenerative eye disease, or a dementia such as frontotemporal dementia (FTD).

As used herein, the terms “treat,” “treatment,” and “treating” refer to a deliberate intervention to a physiological disease state resulting in the reduction in severity of a disease or condition; the reduction in the duration of a disease or condition; the amelioration or elimination of one or more symptoms associated with a disease or condition; or the provision of beneficial effects to a subject with a disease or condition. Treatment does not require curing the underlying disease or condition.

A pharmaceutical composition comprising a TfR-binding protein herein linked to a cargo is useful in treating a human patient with a disorder that benefits from transport of the cargo across the BBB. The cargo may be, e.g., a binding domain targeting a protein of the complement system (e.g., as mentioned in Mastellos et al., supra). In certain embodiments, the protein of the complement system may be C1s, in particular aC1s.

The pharmaceutical composition may be provided to the patient at a dosage strength and a frequency determined as appropriate by a health care provider. Therapeutically effective amounts are those sufficient to ameliorate one or more symptoms associated with the disease or affliction to be treated. A “therapeutically effective amount,” “effective dose,” “effective amount,” or “therapeutically effective dosage” of the binding protein herein protects a subject against the onset of a disease or promotes disease regression or stabilization as evidenced by a decrease in severity of disease symptoms, an increase in frequency and duration of disease symptom-free periods, or a prevention or delay of impairment or disability (e.g., cognitive ability or mobility) due to the disease affliction.

The present disclosure also provides the use of the present binding proteins (e.g., monospecific aC1s- or TfR-binding proteins) for diagnostic processes (e.g., in vitro or ex vivo). For example, the binding proteins can be used to detect and/or measure the level of aC1s or TfR, respectively, in a biological sample from a patient (e.g., a tissue sample such as a brain sample, or a fluid sample such as a blood, plasma, or CSF sample). Suitable detection and measurement methods include immunological methods such as flow cytometry, enzyme-linked immunosorbent assays (ELISA), chemiluminescence assays, radioimmunoassays, and immunohistochemistry. The present disclosure further encompasses kits (e.g., diagnostic kits) comprising the binding proteins described herein.

Unless otherwise defined herein, scientific and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control. Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. All publications and other references mentioned herein are incorporated by reference in their entirety, as if each individual reference were specifically and individually indicated to be incorporated by reference in its entirety. Although a number of documents are cited herein, this citation does not constitute an admission that any of these documents forms part of the common general knowledge in the art. As used herein, the term “approximately” or “about” as applied to one or more values of interest refers to a value that is similar to a stated reference value. In certain embodiments, the term refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context.

According to the present disclosure, back-references in the dependent claims are meant as short-hand writing for a direct and unambiguous disclosure of each and every combination of claims that is indicated by the back-reference. Any compound disclosed herein can be used in any of the treatment method here, wherein the individual to be treated is as defined anywhere herein. Further, headers herein are created for ease of organization and are not intended to limit the scope of the claimed invention in any manner.

In order that this invention may be better understood, the following examples are set forth. These examples are for purposes of illustration only and are not to be construed as limiting the scope of the invention in any manner.

### EXAMPLES

### Example 1: Enhanced Brain Exposure of Anti-aC1s/Anti-TfR Bispecific Antibody Over Monospecific Anti-aC1s Antibody

### Materials and Methods

### Mouse Tissue Radiolabeled Antibody Distribution Study

All animals were handled in accordance with Institutional Animal Care and Use Committee (IACUC) guidelines. Female hTfR knock-in mice ranging from 6 to 12 weeks old and weighing approximately 20 g at the initiation of the study were randomized into groups. All mice received a single oral administration of aqueous potassium iodide (target dose level of 1 mg/kg and a target dose volume of 5 mL/kg) via oral gavage ca. 24 hours and 1 hour prior to administration of each of the [¹²⁵I]-Abs to prevent iodine-125 ([¹²⁵I]) sequestration in the thyroid. The intravenous formulation was administered into the tail vein of the animals using a plastic insulin syringe with pre-attached needle over a slow bolus (ca. 30 seconds) at target dose level of 1 mg/kg and dose volume of 10 mL/kg. The specific radioactivity of [¹²⁵I]-labeled antibodies were 17.4 MBq/kg for anti-aC1s Ab and 16.3 MBq/kg for anti-TfR(V25)-aC1s Ab. Prior to sacrifice, a blood sample was taken from each animal via an orbital sinus bleed. Blood samples were collected for total radioactivity analysis and the remainder centrifuged to obtain plasma. Plasma and red blood cells were then analysed for total radioactivity. The following tissues and organs were harvested terminally at 6, 24, 48, 96 and 168 hours post dose for total radioactivity analysis: brain, cerebrospinal fluid (CSF), spinal cord, sciatic nerve (where possible), liver, kidney, heart, spleen, lung, lymph node, muscle (quadriceps, diaphragm, gastrocnemius) and bone marrow (femur).

Total radioactivity was quantified using an automatic controlled gamma counter (Hidex AMG) with correction for counter efficiency and the radioisotope decay rate ([¹²⁵I]. All samples were counted for 30 seconds. The data for tissues, whole blood, plasma, and red blood cells determined by gamma counting were captured in DEBRA® Management Software (LabLogic Systems Limited, UK). Radioactive counts were converted to dose-normalized concentrations by calculating the percentage of injected dose per gram of tissue (or milliliter of blood or plasma).

### Monkey Radiolabeled Antibody Study

Male cynomolgus monkeys (Cambodian origin) aged 2.5-3 years were housed and handled in accordance with the protocol, the Testing Facility's standard operating protocols, and regulations outlined in the applicable sections of the Final Rules of the Animal Welfare Act regulations (9 CFR), the Public Health Service Policy on Humane Care and Use of Laboratory Animals, and the Guide for the Care and Use of Laboratory Animals. IV dosing of test articles at dose level 1 mg/kg and dose volume 2 mL/kg were administered through the saphenous vein or the cephalic vein with a temporary IV catheter with a bolus injection (over 1-2 minutes) followed by 0.2 mL of saline to flush the dose from the IV catheter. Blood samples were collected from an appropriate peripheral vein (not the vein used for dosing) in K₂EDTA tubes and spun down at 2,000×g at 2° C. to 8° C. for 15 minutes) within 45 minutes of collection before collecting the top plasma layer. CSF samples were obtained from the cisterna magna of the anesthetized animals with an appropriately sized needle (22-23 gauge, ¾″-1.5″) and syringe (i.e. 1-5 mL) via gentle aspiration.

Anti-aC1s Ab and anti-TfR(V25)-aC1s Ab concentrations in monkey plasma and CSF were quantified using a standard 96-well plate Mesoscale Discovery (MSD) assay. Briefly, the plates were coated overnight at 4° C. using recombinant active C1s (Complement Tech, #A104) at 1 μg/mL in 1× DPBS (Invitrogen/Life Tech, #14190-144). Following plate coating, plate blocking with 1× DPBS+1× Casein (Vector Lab, #SP-5020) and wash steps, test samples (standards, QCs, and unknowns) were added to the assay plate and allowed to incubate for 2 hours. Assay diluent was 1× DPBS+0.1% Casein. Following the sample incubation and subsequent wash step, a goat anti-human kappa, monkey ads-BIOT antibody (Southern Biotech, #2064-08) was added at 100 ng/mL and incubated for 1 hour. Following another wash, Streptavidin SULFO-Tag conjugate (MSD, Product No. R32AD-1) at 100 ng/mL was incubated for 40 minutes. After a final wash, 2× Read Buffer (MSD, #R92TC) was added and the plate was read to generate light.

All test samples were pre-diluted at the assay minimum-required-dilution (MRD) of 1:100 for plasma and 1:20 for CSF prior to analysis. The assay standard curves were fitted with a weighted four-parameter (4P) nonlinear logistic regression for use in calculating concentrations of unknown samples

### Mouse Brain Exposure Study

To evaluate brain exposure of anti-TfR binders, hTfR-KI mice were dosed with a single intravenous (tail vein) dose of 70 nmol/kg per IgG molecule (N=9 per group). Control IgG1 corresponds to Southern Biotech's human IgG1 Kappa-LE/AF (Cat. Number 0151K-14). At 1 hour, 3 hours and 24 hours post-dosing, 3 mice from each group were anesthetized with ketamine/xylazine and transcardially perfused with ice-cold heparinated DBPS with Ca/Mg. Brain cortex was harvested and weighted for IgG quantification through MSD Human/NHP IgG kit (Cat. Number K150JLD-4).

Brain tissues were homogenized in 5 v/w of 1% NP-40 in PBS without Ca/Mg, in the presence of protease inhibitors, by mechanical disruption with 2.8 mm ceramic beads in Qiagen's Tissue Lyser LT. Homogenized tissues were centrifuged at maximum speed for 20 minutes at 4° C., and supernatants were collected for IgG quantification.

Brain homogenates were diluted 1/10 in homogenization buffer. MSD plates were blocked with 150 μL of 5% Blocker A (2.5 g of Blocker BSA into 50 mL PBST) per well shaking at RT for 30 min. Standard curve was prepared in 1/10 tissue homogenate from a non-dosed mouse to account for matrix effect. 25 μL of each standard or experimental sample were loaded per well, and incubated shaking at RT for 2 hours. Plates were washed with 200 μL/well of PBST 3 times. 50× detection antibody (SULFO-TAG Anti-Hu/NHP IgG) was diluted with Diluent 100 to 1×. 25 μL of detection antibody were added into each well and incubated shaking at RT for 2 hours. Plates were washed with 200 μL/well of PBST 3 times. 2× Read Buffer T (from 4×, dilute to 1/2 with water) was prepared and 150 μL were added to each well. Plates were read immediately.

IgG concentration per tissue was calculated upon extrapolation from the standard curve, considering the dilutions of the loaded brain homogenate.

### Results

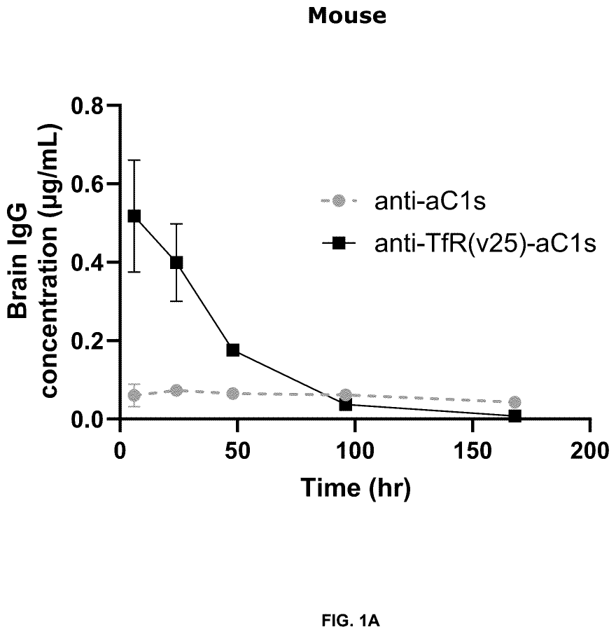

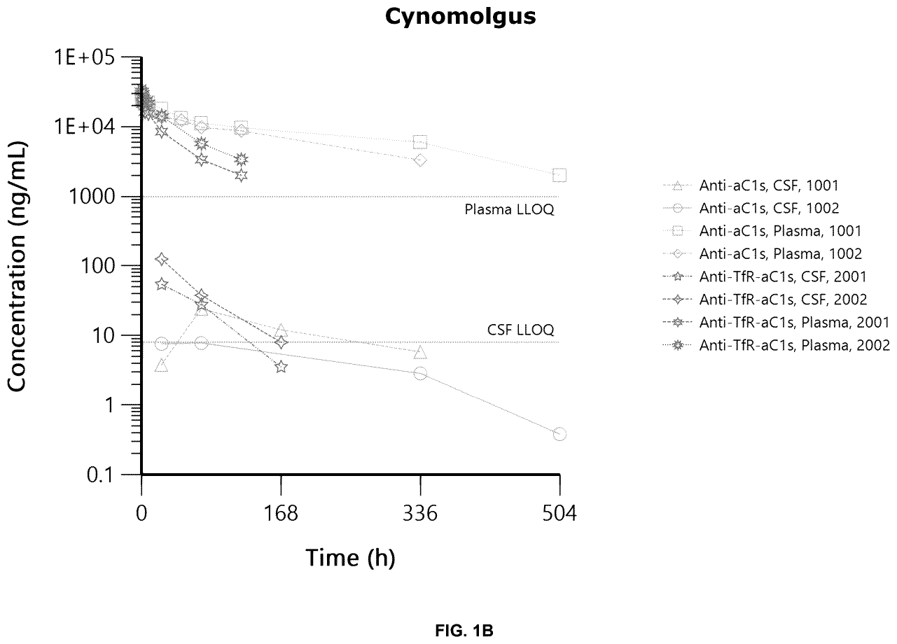

To test the pharmacokinetics and biodistribution of an anti-aC1s/anti-TfR bispecific antibody, humanized TfR mice were dosed intravenously (1 mg/kg) with ¹²⁵I radiolabeled IgGI antibodies: an anti-aC1s monospecific antibody (SAR 445088) or an anti-aC1s/anti-TfR bispecific antibody (anti-aC1s: SAR 445088; anti-TfR: 531v25). The brains were harvested for measurement of antibody concentration in the brain tissue at several timepoints post injection (6, 24, 48, 96 and 168 h) (FIG. 1A). The bispecific antibody showed enhanced brain exposure compared to the monospecific anti-aC1s antibody.

Additionally, cynomolgus monkeys were intravenously injected (1 mg/kg) with radiolabeled anti-aC1s monospecific and anti-aC1s/anti-TfR bispecific antibodies. Fluids were harvested at predose, 0.25, 1, 4, 8, 24, 48, 72, 120, 336, and 504-hours post-dose for plasma and Day −2, 24, 27, 168, 336, and 504-hours post-dose for CSF respectively and the concentration of each antibody measured (FIG. 1B). The bispecific antibody exhibited enhanced CSF exposure compared to the monospecific anti-aC1s antibody.

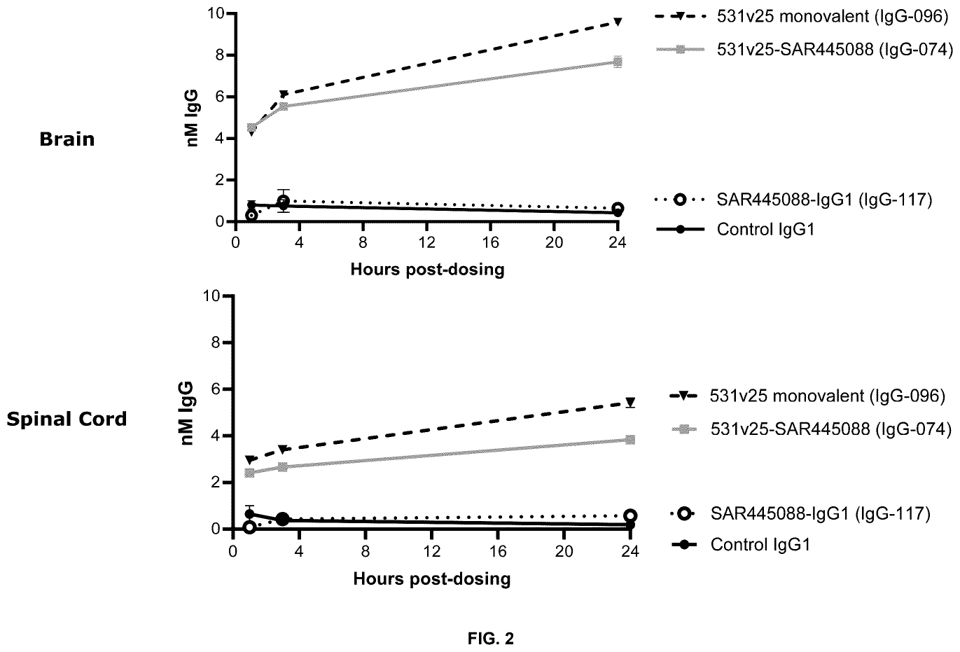

To further demonstrate anti-aC1s/anti-TfR bispecific antibody brain exposure, humanized TfR mice were dosed with several antibodies: a control IgG1 antibody, an anti-TfR monovalent antibody (IgG-096), an anti-aC1s bivalent antibody (IgG-117), or an anti-aC1s/anti-TfR bispecific antibody (IgG-074). The tested antibodies each comprised NNAS mutations in a human IgG1 Fc domain, and antibodies IgG-074 and IgG-096 each also comprised RF and dKiH mutations. At 1, 3, and 24 hours post-injection, the brain and spinal cords were harvested and the concentration of the antibodies measured (FIG. 2). The bispecific IgG-074 demonstrated concentrations in both samples comparable to the monovalent anti-TfR antibody (IgG-096) and significantly higher than the non-TfR antibodies, indicating the efficacy of the anti-TfR shuttle.

### Example 2: Anti-TfR Shuttle Optimization

### Materials and Methods

### Mouse Exposure Study

All experiments were conducted according to the NIH guidelines for animal research and were approved by the Sanofi Institutional Care and Use Committee. hTfR-KI male mice were group-housed on a 12-hour light-dark cycle and acclimated for 72 hours before use in experiments.

Prior to tail IV injection, each mouse was carefully weighed to ensure accurate dosage calculation. The volume of the injection was determined based on the mouse's body weight, with a maximum limit of 1% of the body weight, not exceeding 0.2 mL per injection. These weights and corresponding agent volumes were meticulously recorded for each animal to ensure precision in dosing.

The animals were warmed for a period of 5-10 minutes to facilitate vein dilation. This warming was achieved by placing the animals a commercially available warming box (Mini Thermacage with Diffuser, Braintree Scientific, INC. #CS7A04A). This step was crucial to ensure that the veins were adequately dilated, allowing for smoother and more efficient administration of the injection.

At the time of the injection, mice were placed in a Tailveiner Restrainer for Mice (Braintree #TV-150 LG), and sterile 27-gauge needles were used in conjunction with 1 mL syringes. The anti-TFRC/aC1s agent and relative control were administered via lateral tail injections, ensuring that each animal received the correct dosage based on its previously recorded weight.

Throughout the procedure, care was taken to follow best practices and maintain a sterile environment, ensuring the well-being of the animals and the integrity of the experimental results. Following the injections, the mice were euthanized with carbon dioxide for 5 minutes and transcardially perfused with PBS at different time points. Blood was harvested in EDTA tubes and centrifuged for 5 minutes at 10,000 RPM. Plasma was collected and stored at −80° C. until further processing. Brains were harvested and both hemispheres were flash frozen on dry ice and stored at −80° C. until further processing.

Frozen brain hemispheres were transferred into Pre-Filled Bead Mill Tubes (Fisher #15-340-154) containing 1× RIPA lysis buffer (Cell Signaling, 9803) and 1× Halt™ Protease and Phosphatase Inhibitor Cocktail (Thermo Scientific #PI78440). Subsequently, they were homogenized using Bead Ruptor 24™ Bead Mill Homogenizer (Omni International) at a 1:5 (w/v) dilution (e.g., 100 mg tissue in 500 μL prefilled lysis buffer). The tissue lysate was centrifuged at maximum speed (14,000 RPM) for 10 minutes, then the supernatant was transferred into a new tube for further processing and stored at −80° C., and the pellet was discarded.

For IgG measurement, the MSD Human/NHP Isotyping kit was utilized (Meso Scale Discovery #K15203D). Prior to IgG measurement by MSD, plasma samples were diluted to 1:500 in diluent 100 provided by the ELISA kit, while brain lysate was run without dilution. Dilutions were adjusted based on the concentration of the injected antibody. For example, 10 mg/kg would require a 10-fold dilution for brain lysates, 5 mg would require a 5-fold dilution, and so on. All reagents and materials were supplied by the kit, and the assay was conducted following the provided protocol. Following the assay, plates provided by the kit were promptly read with the MSD Meso-Sector S600 plate reader and analyzed using the MSD Discovery Workbench.

### TfR Active Concentration

The active concentration of bispecific antibodies was tested at 0 weeks, 1 week, 2 weeks, and 3 weeks by capturing the antibodies to an anti-Fc immobilized CM5 Series S sensor chip on a Biacore™ T200. A single concentration (45 nM) of human TfR was then injected over the sensor surface and a report point was taken for the binding response. The surface was regenerated for subsequent injections. Bispecific antibodies were injected in triplicate at each indicated time point. The binding response of each injection was normalized to the capture level of each bispecific antibody and the loss per week was calculated based off the first time point.

### Results

To compare the brain exposure of the bispecific antibody, several variants of the anti-TfR shuttle portion were tested in combination with the anti-aC1s antibody SAR 445088 (with charge mutations of Q39E in the VH and Q42K in the VL). Three IgG4-based bispecific antibodies were tested for their molecular stability as a measure of the active concentration lost per week and their affinity for hTfR (Table 1).

TABLE 1
Molecular Stability and Affinity of Bispecific
Antibodies with Different Anti-TfR Arms

| Molecule Stability | | | hTfR |
|---|---|---|---|
| (active concentration loss/week) | | | Affinity |
| Molecule | pH 5.0 | pH 8.0 | kD (nM) |
| aC1s_088-CM × 531v25.v6 IgG4 | +1.2% | −2.5% | 18.9 |
| aC1s_088-CM × 531v23 IgG4 | 0.78% | −1.5% | 9.65 |
| aC1s_088-CM × 531v25 IgG4 | −0.03% | −5.2% | 22.6 |

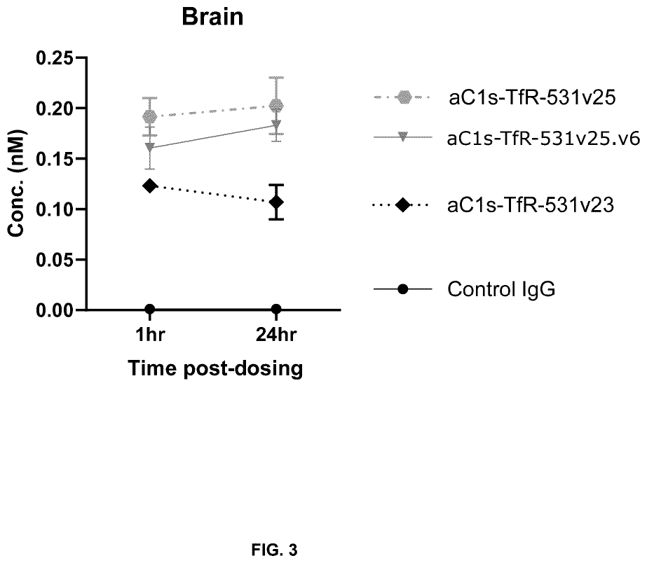

Humanized TfR mice were injected intravenously with 1 mg/kg of each bispecific antibody and the concentration of antibody in the brain tissue was measured 1 hr and 24 hrs post injection (FIG. 3). All three bispecific antibodies exhibited improved brain exposure compared to an IgG4 control antibody. Antibody 531v25.v6 was selected for further studies based on exposure and stability.

To further optimize the anti-TfR shuttle portion of the bispecific antibody, the three anti-aC1s/anti-TfR variants were designed with either IgG1-NNAS or IgG4-PE Fc domains and charge pairing (CP) mutations.

The charge pairing mutations markedly improved cognate LC pairing compared to wild-type. The six antibodies with charge pairing mutations were selected for further functional and biodistribution studies.

### Example 3: Anti-TfR Shuttle Functional Assessment

### Materials and Methods

### Antibody Binding

TfR and C1s binding using surface plasmon resonance (SPR): Analysis of antibody binding to TfR was performed on a Biacore™ T200 instrument using anti-His capture at 25° C. Anti-His (Qiagen) was buffer exchanged into PBS pH 7.2, diluted to 25 μg/mL in 10 mM sodium acetate pH 4.0 and directly immobilized to a series S CM5 chip to a surface density of ˜10,000 RU using the amine coupling kit provided by Cytiva. His-tagged recombinant human TfR (produced in-house) was diluted in HBS-EP+ pH 7.4 running buffer (10 mM HEPES pH 7.4, 150 mM NaCl, 3 mM EDTA, 0.05% surfactant P20) and injected for 5 sec at 20 μL/min flowrate to obtain capture level of 20 RU. Antibodies were serially diluted 2-fold from 90 to 5.625 nM in running buffer and injected over the captured TfR for 3 min followed by 9 min dissociation in buffer at 30 μL/min. Each cycle was regenerated with 10 mM glycine pH 1.5. To measure C1s binding, anti-human Fc (Jackson) was directly immobilized to a CM5 chip using amine chemistry. Antibodies were diluted to 2.5 μg/mL in HBS-EP+ pH 7.4 and captured to the chip for 15 sec at 20 μL/min flowrate. Human active C1s (Complement Technology) was serially diluted 2-fold from 20 to 0.625 nM in running buffer and injected over the captured antibodies for 180 sec at 50 μL/min followed by 9 min dissociation in buffer. The bound complex was removed from the chip each cycle with 40 mM HCl and 0.85% phosphoric acid. Sensorgrams were processed using the Biacore™ Insight software and fit to a 1:1 binding model to obtain kinetic constants.

### Wieslab Complement Pathway Assay

The potency of the bispecific anti-aC1s/anti-TfR antibodies to assess complement inhibition were performed using commercially available Wieslab Classical Pathway EIA kits (Svar Life Science, Catalog No. COMPL CP 310) in pooled normal human serum (Complement Technology, Catalog No. NHS) as per the manufacturer's instructions. The bispecific anti-aC1s/anti-TfR antibodies were serially diluted 4-fold in Diluent CP and mixed with Normal Human Serum to achieve a final concentration of 200 ng/mL to 0.048 ng/mL in 1% Normal Human Serum. Blank, positive control (PC) and negative control (NC) were prepared per the manufacturer's instructions. 100 μL of the diluted samples were added to the coated assay wells of CP ELISA plate. Complement activation was allowed to occur for 1 hour at 37° C. After serum incubation, the plate wells were washed, and 100 μL of conjugate containing alkaline-phosphatase-labeled antibodies specific to a neoepitope on MAC was added to each well of the assay plates. The incubation was carried out at room temperature for 30 minutes followed by a final wash step. The anti-MAC antibodies that remained bound were quantified by incubation with an alkaline phosphatase substrate solution at room temperature for 30 minutes. The absorbance of solution in the wells were read on a SpectraMax® i3 plate reader at 405 nm.

The Blank absorbance was subtracted from the absorbance of the samples, PC and NC. Percent Complement activity was calculated according to the formula below:

$$( ( {OD}_{sample} - OD NC ) / ( ODPC - ODNC ) ) * 100$$

The percent complement activity was plotted as a function of antibody concentration in GraphPad Prism (v10) software. The half maximal inhibitory concentration (IC₅₀) was determined from the dose-response curves.

### Results

The six bispecific anti-aC1s/anti-TfR antibodies were first tested for their affinity for both TfR and aC1s via surface plasmon resonance (SPR) (Table 2). The 531v23 variants exhibited roughly two times higher affinity for TfR compared to the 531v25 and 531v25.v6 variants. Additionally, the aC1s affinities did not appear to be influenced by the anti-TfR arm with which the anti-aC1s arm was paired.

TABLE 2
Binding Affinity of Bispecific Anti-TfR Antibodies

| | TfR | | | aC1s | | |
|---|---|---|---|---|---|---|
| | kₐ | k_{d} | K_{D} | kₐ | kₐ | K_{D} |
| Protein Name | (1/Ms) | (1/s) | (nM) | (1/Ms) | (1/s) | (nM) |
| aC1s_088 × 531v23 IgG4-PE/dK | 1.89E+05 | 1.83E−03 | 9.65 | 3.14E+06 | 2.45E−03 | 0.79 |
| aC1s_088 × 531v23 IgG1-NNAS/dK | 1.92E+05 | 1.97E−03 | 10.3 | 4.18E+06 | 2.75E−03 | 0.66 |
| aC1s_088 × 531v25 IgG4-PE/dK | 1.83E+05 | 4.13E−03 | 22.6 | 3.31E+06 | 2.46E−03 | 0.75 |
| aC1s_088 × 531v25 IgG1-NNAS/dK | 1.99E+05 | 4.90E−03 | 24.6 | 4.22E+06 | 2.85E−03 | 0.68 |
| aC1s_088 × 531v25.v6 IgG4-PE/dK | 1.61E+05 | 3.04E−03 | 18.9 | 3.59E+06 | 2.73E−03 | 0.76 |
| aC1s_088 × 531v25.v6 IgG1-NNAS/dK | 1.64E+05 | 3.32E−03 | 20.3 | 3.96E+06 | 2.72E−03 | 0.69 |

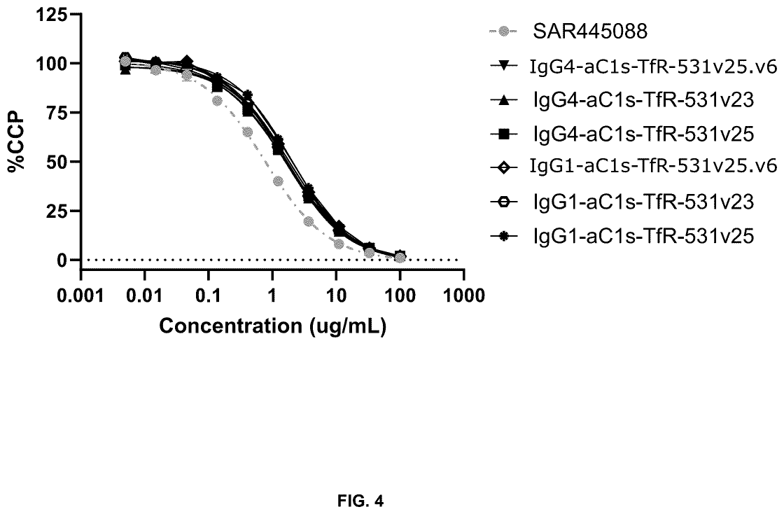

To further characterize the classical complement pathway inhibitory function of the bispecific antibodies, a Wieslab Complement Pathway Assay was conducted to determine potency in comparison with the SAR445088 monospecific antibody (Table 3, FIG. 4). All six antibodies inhibited the complement pathway, albeit with a small potency loss in line with the anti-aC1s valency of the antibody.

TABLE 3
IC₅₀ Values of Bispecific Anti-TfR Antibodies

| Variants | IC₅₀ (μg/mL) |
|---|---|
| SAR445088 | 0.77 |
| aC1s_088 × 531v25.v6 IgG1-NNAS/dK | 1.78 |
| aC1s_088 × 531v25.v6 IgG4-PE/dK | 1.79 |
| aC1s_088 × 531v23 IgG1-NNAS/dK | 1.67 |
| aC1s_088 × 531v23 IgG4-PE/dK | 1.67 |
| aC1s_088 × 531v25 IgG1-NNAS/dK | 2.02 |
| aC1s_088 × 531v25 IgG4-PE/dK | 1.63 |

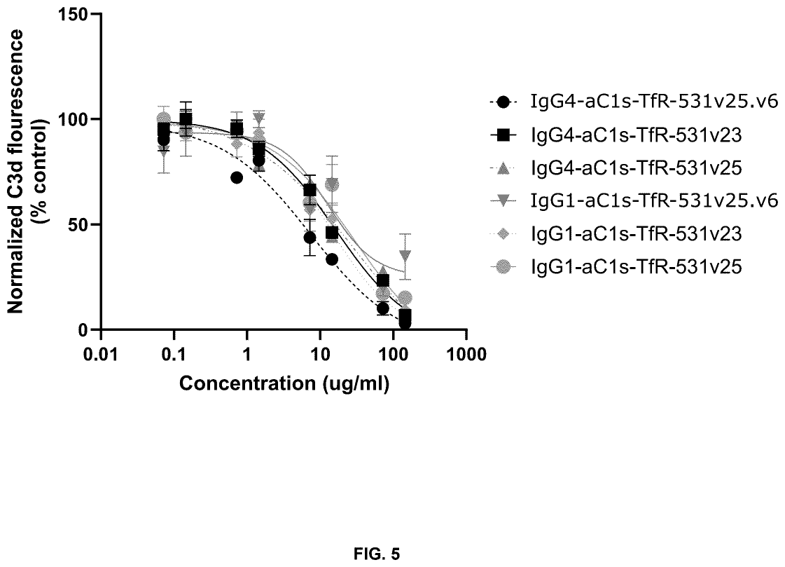

To further assess the potency of the bispecific anti-aC1s/anti-TfR antibodies in a brain relevant human system, induced pluripotent stem cell brain tri-cultures (neurons, microglia, and astrocytes) were treated with either 3% serum or 3% serum and the bispecific antibody at various concentrations. The cells were imaged and the complement activity was measured by the levels of C3d fluorescence colocalized with neurons (Table 4, FIG. 5). The antibodies all showed a dose-dependent inhibition of complement deposition on the neurons in the tri-culture.

TABLE 4
IC₅₀ Values of Bispecific Anti-TfR Antibodies in iPSC Tricultures

| Variants | IC₅₀ (μg/mL) |
|---|---|
| aC1s_088 × 531v25.v6 IgG1-NNAS/dK | 12.75 |
| aC1s_088 × 531v25.v6 IgG4-PE/dK | 7.48 |
| aC1s_088 × 531v23 IgG1-NNAS/dK | 13.95 |
| aC1s_088 × 531v23 IgG4-PE/dK | 16.24 |
| aC1s_088 × 531v25 IgG1-NNAS/dK | 29.71 |
| aC1s_088 × 531v25 IgG4-PE/dK | 29.3 |

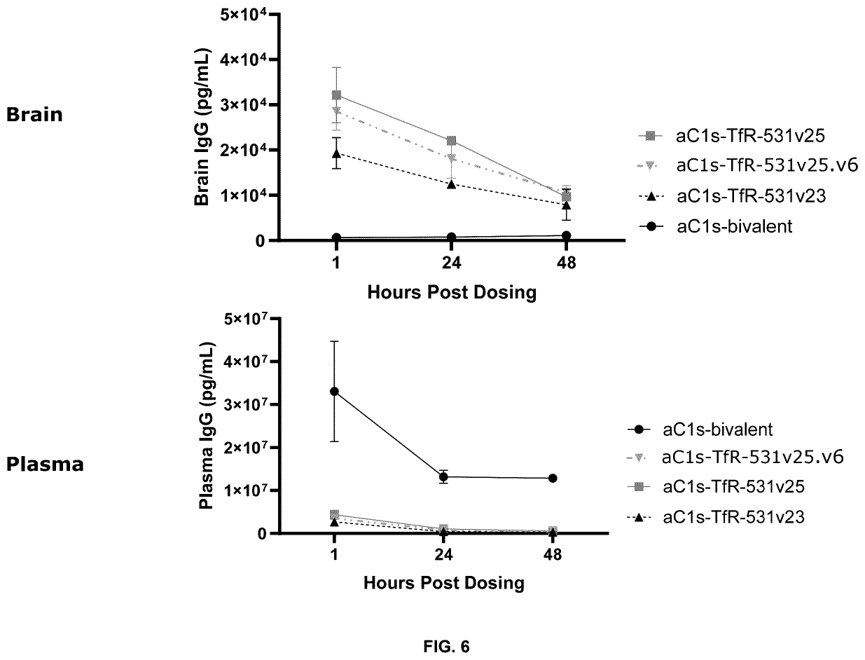

Finally, to test the brain trafficking and biodistribution of the bispecific antibodies in vivo, the three variants comprising IgG1-NNAS Fc domains and charge pairing mutations were injected into humanized TfR mice. One of the three variants, or an anti-aC1s control antibody (bivalent SAR445088), was intravenously injected (1 mg/kg) into each mouse (3 mice/group) and tissue samples collected after 1, 24, and 48 hours. The concentration of antibody in the brain and plasma were measured at each time point (FIG. 6). The bispecific antibodies each showed enhanced brain exposure compared to the anti-aC1s bivalent antibody, with the v23 variant exhibiting the highest affinity for hTfR and lowest brain exposure.

### Example 4: Anti-aC1s Optimization

### Materials and Methods

### Antibody Binding

C1s binding using surface plasmon resonance (SPRi): Analysis of active C1s binding was performed on a Carterra LSA SPRi instrument at 25° C. Protein A/G (Sigma) diluted in 25 mM MES pH 6.0, 0.05% Tween-20 running buffer was covalently immobilized to an HC200M sensor chip using amine coupling. Antibodies were diluted to 0.2 μg/mL in HBS-EP+ buffer and printed in duplicate to the protein A/G surface in the capture 96-array format for 5 min followed by a 1 min baseline in buffer. Human active C1s (Complement Technology) was serially diluted 3-fold from 1 μM to 0.781 nM in running buffer and injected over the captured antibodies for 5 min with a 5 min dissociation time. The surface was regenerated with 10 mM glycine pH 1.5 with two 30 sec injections. Sensorgrams were processed with the Carterra® K.I.T. Inspection Tool and fit to a 1:1 binding model to obtain kinetic constants.

### Wieslab Complement Pathway Assay

The assay was performed as described in Example 3.

### Mouse Pharmacokinetics Using Ligand Capture Assay

The plasma protease activated C1s (Complement Tech, A104, Lot 33b) was diluted to 2.5 μg/mL in 1× PBS pH 7.4 (Gibco, Ref. No. 10010-023, Lot 2561363). 50 μL of the diluted aC1s was added to each well of a MaxiSorp™ high binding 96 well flat bottom plate (Thermo Scientific Nunc, Cat. No. 439454) and incubated overnight at 2-8° C. to coat the wells. After overnight incubation, the coating solution was aspirated from the plate wells and 300 μL of wash buffer (0.05% Tween 20 in 1× PBS) was added to each well. The wash step was repeated for a total of 4 washes using Biotek ELX405SELECTCW automated plate washer. 300 μL of blocking buffer (1× casein in 1× PBS) was added to each well and the plates were incubated at room temperature for 2 hours. Brain lysates and plasma collected from dosed hTfR KI mice were diluted in assay buffer (0.1× casein in 1× PBS). The brain lysates were diluted in the range of 30-fold to 80-fold and plasma in the range of 15000-fold to 60000-fold based on the timepoint and dosage. The drug a-TFRC_531v25.v6_CM1 x a-C1S_L4_D32E_CM2, CR3, NN3)-huIgG4_PE_LS_dK (aC1s_D32E-CM2/CR3/NN3 x 531v25.v6-CM1 IgG4-PE/LS/dK) and SAR445088 serially diluted 2-fold from 100 ng/mL to 0.097 ng/mL in assay buffer was used as the standard curve.

The plates were washed with wash buffer as described above for a total of 4 washes. 50 μL of the diluted samples and standards were added to each well and the plates were incubated at room temperature for 1 hour with constant agitation of 400 rpm.

Detection of the drug was carried out using goat anti-human kappa, Mouse ads-HRP detection antibody (Southern Biotech, Cat. No. 2061-05, Lot I1519-T292) diluted to 5000-fold in assay buffer. The plates were washed with wash buffer as described above for a total of 4 washes, and 50 μL of detection antibody was added to each well of the assay plates. The incubation was carried out at room temperature for 80 minutes at 400 rpm followed by a final wash of the plates with 1× PBS for a total of 4 washes.

QuantaRed™ Chemifluorescent HRP working solution (Thermo Scientific, Ref. No. 15159) was prepared as per manufacturer's instructions by mixing 50 parts QuantaRed™ Enhancer Solution with 50 parts QuantaRed™ Stable Peroxide and 1 part of the QuantaRed™ ADHP Concentrate. 100 μL of QuantaRed™ working solution was added to each well of the assay plate and color development was allowed to proceed for 10 minutes at room temperature. The reaction was stopped by the addition of 10 μL of QuantaRed™ stop solution. 95 μL of the reaction volume was transferred to black, clear bottom plates for fluorescent measurement.

The relative fluorescence units of solution in the wells were read on a SpectraMax i3 plate reader at 530 nm excitation and 585 nm emission. The Mean RFUs were plotted as a function of antibody concentration for the standard curve, in GraphPad Prism (v10) software. The data were then fit to the log (agonist) versus response-four parameters function, described by the equation:

$$Y = Bottom + ( ( ( Top - Bottom ) ) / ( ( 1 + 10 \hat{} ( ( ( Log EC 50 - X ) * Hillslope ) ) ) ) )$$

The linear range of the standard curve was determined to be 25 ng/mL-1.563 ng/mL. The antibody concentration for the brain lysates and plasma were interpolated from their respective standard curve. The antibody concentration for the brain lysates was normalized to protein concentration to express the drug levels in ng/mg.

### Results

To further improve the potency of the anti-aC1s/anti-TfR bispecific antibodies, the anti-aC1s arm was optimized. SAR445088 (“the wild-type antibody”) was engineered to remove an isomerization hotspot on the light chain (D32), close to a sulfated tyrosine (Y36). The DG (and the adjacent DS) motif was engineered to remove the isomerization hotspot. Sulfation on tyrosine 36 was not targeted directly and was predicted to disappear upon modification of the adjacent DGDS motif for some variants.

Three variants were selected as viable engineered alternatives to the wild-type anti-aC1s sequence: D32E, D32S, and D32Y. The three variants were expressed as bivalent monospecific anti-aC1s antibodies with human IgG1 and their affinity for aC1s tested by surface plasmon resonance (Table 5). All three variants fixed the isomerization hotspot (D32) and had comparable expression levels to WT. The D32S and D32Y variants did not show sulfation on Y36. The D32E and D32Y variants had similar affinity for aC1s as the wild-type antibody, while D32S had a slightly reduced affinity.

TABLE 5
Binding Affinity of Monospecific Anti-aC1s Antibodies

| | Surface Plasmon Resonance | | | |
|---|---|---|---|---|
| Variant* | kₐ (1/Ms) | k_{d} (1/s) | K_{D} (nM) | Y36 Sulfation |
| D32 (WT) | 1.3E+06 | 3.3E−03 | 2.5 | Yes |
| D32E | 1.3E+06 | 3.2E−03 | 2.5 | Yes |
| D32S | 8.9E+05 | 6.5E−03 | 7.3 | No |
| D32Y | 1.0E+06 | 2.5E−03 | 2.4 | No |

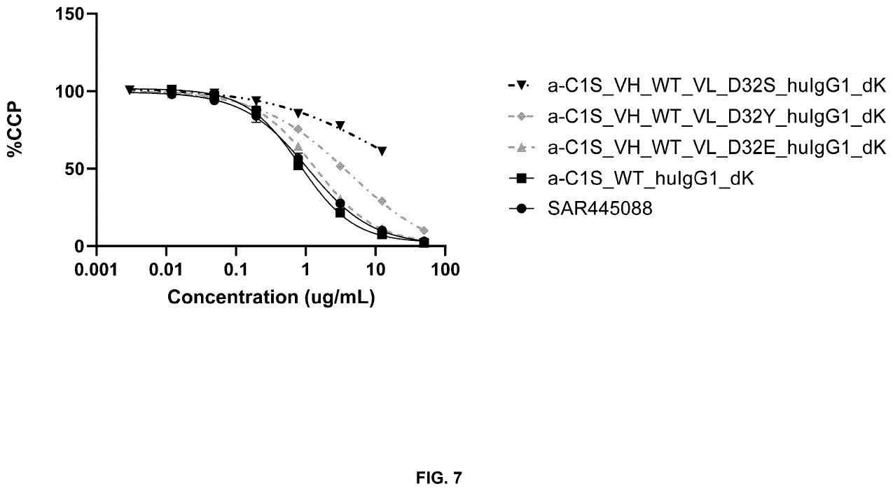

To further characterize the aC1s D32 variants, the bivalent monospecific anti-aC1s antibodies were characterized by the Wieslab Complement Pathway Assay (Table 6, FIG. 7). The D32E and D32Y variants both exhibited complement inhibition while removing one or both binding site liabilities. The D32S variant exhibited low potency.

TABLE 6
IC₅₀ Values of Bivalent Anti-aC1s Monospecific Antibodies

| Variants | IC₅₀ (μg/mL) |
|---|---|
| SAR445088 (BIVV020) | 1.07 |
| aC1s_088 IgG1-dK | 0.84 |
| aC1s_D32E IgG1-dK | 1.36 |
| aC1s_D32S IgG1-dK | 10.74 |
| aC1s_D32Y IgG1-dK | 4.18 |

As a result of these studies, six variants of the D32E anti-aC1s variant were designed, and their binding affinity to aC1s assessed in Fab format (Table 7).

TABLE 7
Anti-aC1s Lead Variants

| | | | | aC1s Binding | |
|---|---|---|---|---|---|
| VL | SEQ | VH | SEQ | Domain | aC1s kD (nM) |
| D32E | 21 + D32E | WT | 10 | | 9.93E−10 |
| D32Ev2 | 18 | WT | 10 | L2 | 8.54E−10 |
| D32Ev4 | 14 | WT | 10 | L4 | 6.54E−10 |
| D32E | 21 + D32E | v5 | 12 | | 4.15E−10 |
| D32Ev2 | 18 | v5 | 12 | L2H5 | 4.67E−10 |
| D32Ev4 | 14 | v5 | 12 | L4H5 | 2.27E−10 |

SEQ: SEQ ID NO:
21 + D32E: SEQ ID NO: 21 wherein residue D32 is substituted with E

These anti-aC1s binding domains were then combined with the 531v25.v6 anti-TfR shuttle arm. The resulting antibodies were purified from CHO cells and tested for their affinity for both human and cynomolgus aC1s and TfR (Table 8). All variants exhibited similar affinities for TfR and aC1s.

TABLE 8
Binding Affinity (kD, nM) of Bispecific
Anti-aC1s/Anti-TfR Variants

| Molecule | Human TfR | Cyno TfR | Human aC1s | Cyno aC1s |
|---|---|---|---|---|
| L4H5 | 39.3 | 72.5 | 2.26 | 0.252 |
| L4 | 28.6 | 43.7 | 3.31 | 0.248 |
| L2H5 | 34.3 | 48.9 | 2.64 | 0.315 |
| L2 | 29.9 | 52.8 | 3.19 | 0.243 |

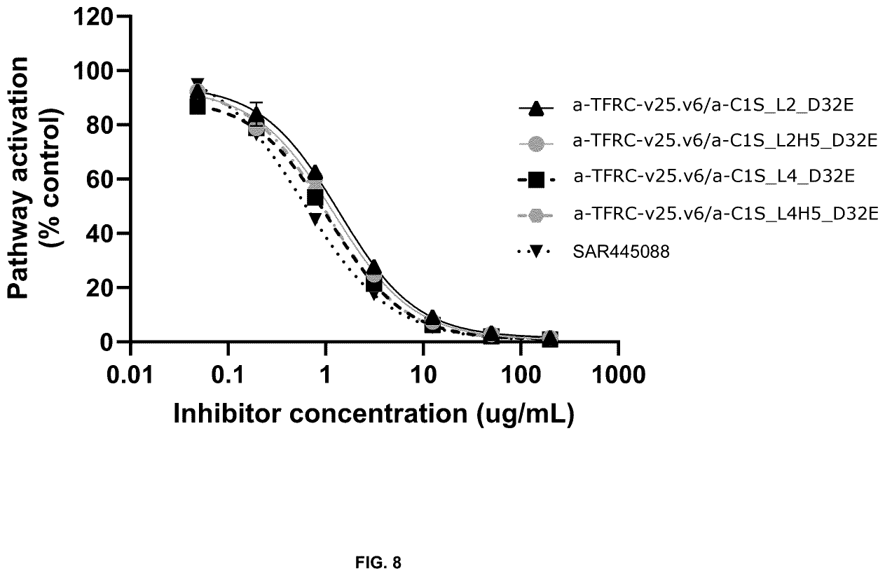

To test the potency of the re-engineered variants, a Wieslab Complement Pathway Assay was conducted on the bispecific anti-aC1s/anti-TfR variants in comparison to the anti-aC1s bivalent antibody SAR445088 (Table 9, FIG. 8). The variants demonstrated enhanced potency that was comparable to the SAR445088 monospecific antibody, despite loss of valency.

TABLE 9
IC₅₀ and IC₉₀ Values of Bispecific Anti-aC1s Antibodies

| Molecule | aC1s valency | IC₅₀ (μg/mL) | IC₉₀ (μg/mL) |
|---|---|---|---|
| SAR445088 | bivalent | 0.45 | 5.53 |
| (batch 1) | | | |
| SAR445088 | bivalent | 0.63 | 7.77 |
| (batch 2) | | | |
| L4H5 | monovalent | 1.04 | 7.55 |
| L4 | monovalent | 1.08 | 7.60 |
| L2H5 | monovalent | 1.23 | 9.70 |
| L2 | monovalent | 1.37 | 10.18 |

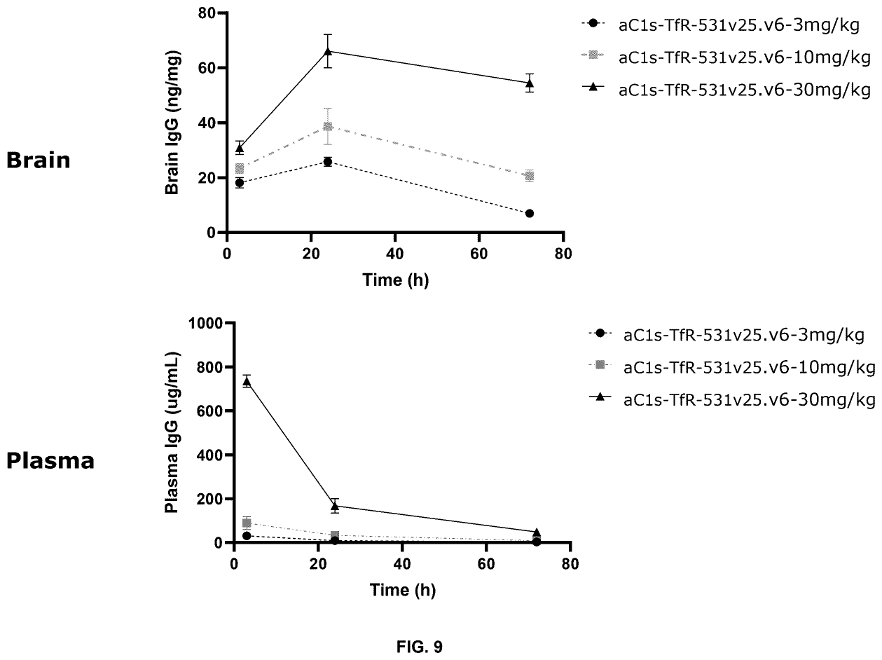

To assess the brain exposure of the bispecific anti-aC1s (L4)/anti-TfR (531v25.v6) IgG4 antibody, human TfR knock-in mice were intravenously injected with various doses of the antibody (3 mg/kg, 10 mg/kg, and 30 mg/kg) and tissues collected at several timepoints post-injection: 3, 24, and 72 hours. At each timepoint, the concentrations of antibody in the brain and plasma of the mice were measured (FIG. 9). The bispecific antibody exhibited enhanced brain exposure at all dosages, and low levels in plasma.

### Example 5: Tissue Exposure and iPSC Brain Triculture for Bispecific Anti-aC1s/Anti-TfR Antibody

### Materials and Methods

### Dosing and Material Collection in Monkeys

Male cynomolgus monkey (Mauritius origin) aged 3-8 years and with a weight range of ˜3 to 6 kg were housed and handled in accordance with UL Lafayette-NIRC SOPs and in accordance with the regulations outlined in the USDA Animal Welfare Act (9 CFR, Parts 1, 2 and 3) and the conditions specified in The Guide for Care and Use of Laboratory Animals (ILAR publication, 1996, National Academy Press). Intravenous (IV) dosing of test articles at dose levels of 10 and 30 mg/kg and dose volume of ˜5 mL/kg were administered at an infusion rate of 2.5 mL/min followed by 0.5 mL of PBS to flush the dose from the IV catheter. Blood samples were collected from an appropriate peripheral vein and spun down under refrigeration to obtain plasma. Serum blood samples were collected from each animal into SST Vacutainer® tubes and allowed to clot at room temperature for no more than 45 minutes following collection and were processed to serum (2,000×g at 2° C. to 8° C. for 15 minutes). CSF samples were obtained from the cisterna magna or lumbar puncture of the anesthetized animals.

For brain tissue collection, animals were sedated with ketamine (10 mg/kg IM) and transferred to the necropsy suite. The hair was clipped from the animal's thoracic and cranial region, and an indwelling catheter was placed in a peripheral vein. A lethal dose of sodium pentobarbital was administered at ˜0.5 mL/kg to effect until the animal became a reflexive. The thoracic cavity and then the pericardial sac were opened to expose the heart. The outer wall of the right atrium was incised with small scissors. The tip of the infusion needle was immediately inserted through the inferior wall of the left ventricle into the ventricular cavity, and the flow of saline (cold PBS containing heparin, 1000 U/L) was turned on. The needle was clamped in place with a small hemostat being sure that the needle tip was placed in the cavity and not in the heart muscle. The animal body was flushed until the solution draining from the atrium was considered clear by visual inspection. The brain was then removed and sectioned, and then frozen for further processing.

### aC1s Capture Assay

Anti-aC1s (L4)/anti-TfR (531v25.v6) IgG4 antibody concentration in monkey plasma, CSF and tissue (brain, spinal cord, sciatic nerve, and gastrocnemius muscle) was quantified using a standard 96-well plate Mesoscale Discovery (MSD) assay. Briefly, the plates were coated overnight at 4° C. using recombinant active C1s (Complement Tech, #A104) at 1 μg/mL in 1× DPBS (Corning, #21-031-CV). Following plate coating, plate blocking with 5% MSD Blocker A and wash steps, test samples (standards, QCs, and unknowns) were added to the assay plate and allowed to incubate for 2 hours. Assay diluent was 1% MSD Blocker A. Following the sample incubation and subsequent wash step, a Goat anti-human Kappa, Monkey ads-BIOT antibody (Southern Biotech, #2064-08) at 100 ng/mL was added and incubated for 1 hour. Following another wash, Streptavidin SULFO-Tag conjugate (MSD, Product No. R32AD-1) at 100 ng/mL was incubated for 40 minutes. After a final wash, 2× MSD Read Buffer T (MSD, #R92TC-1) was added and the plate was read to generate light. All test samples were pre-diluted at the assay minimum-required-dilution (MRD) of 1:100 for plasma and 1:20 for CSF/tissue prior to analysis. The assay standard curves were fitted with a 5 PL (MARQUARDT) fit with weighting factor of 1/y2 for use in calculating concentrations of unknown samples.

### Complement Tri-Culture Deposition Assay/IC₅₀ for Bispecific TfR-aC1s Antibody

Human iPSC motor neurons, microglia, and astrocytes from Fujifilm cellular dynamics were plated sequentially as described in Ryan et al., Nature Neuroscience (2023) 26:12-26. The cultures were then treated with 3% complement-preserved serum alone (positive control, Quidel, A113) or 3% serum plus TfR-aC1s antibody (concentration range: 0.1-400 μg/mL) for 2 h. Cells were then fixed with 4% PFA and stained with primary antibodies against: Beta III tubulin (TUJ1, Novus Biologicals, NB100-1612; 1:500), C3d (Dako/Agilent Rabbit anti C3d, A0063; 1:250) and DAPI (Life Technologies, D3571; 1/5000) overnight at 4° C. Secondary antibodies: Alexa Fluor Goat 488 (C3d), and 647 (TUJ1) (Thermo Fisher; 1/500). Representative images from control, 3% serum, and 3% serum+TfR-aC1s conditions (n=9).

### Results

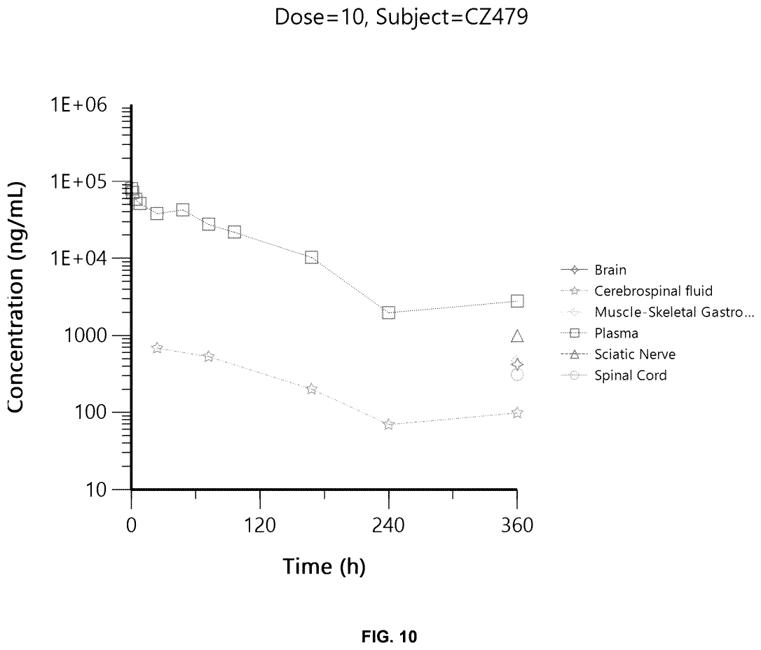

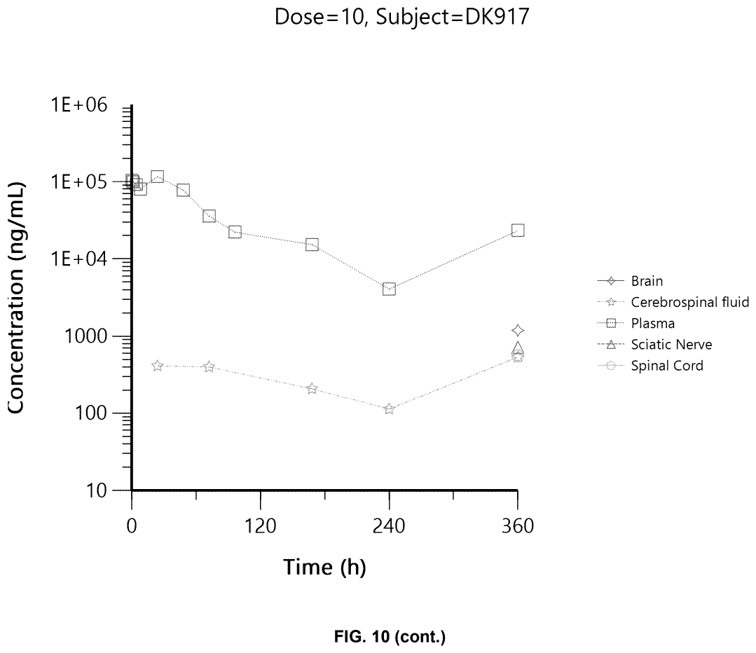

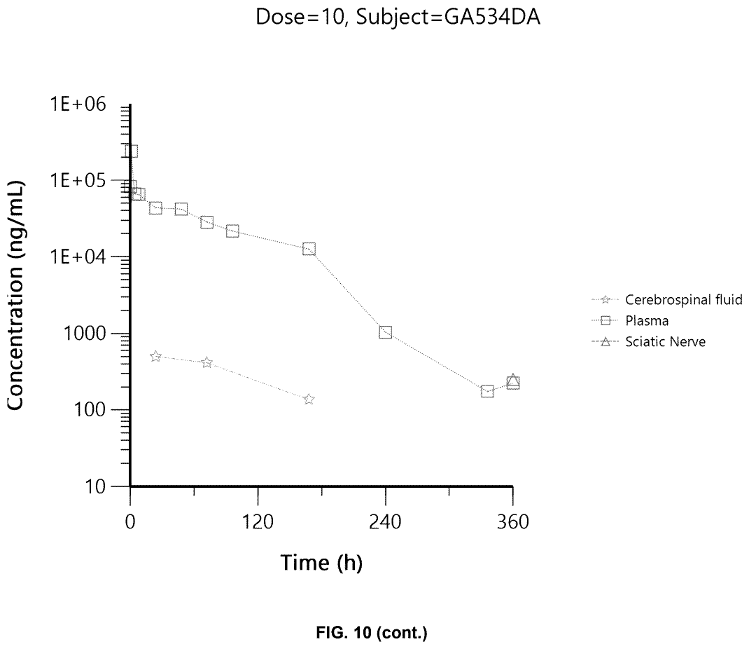

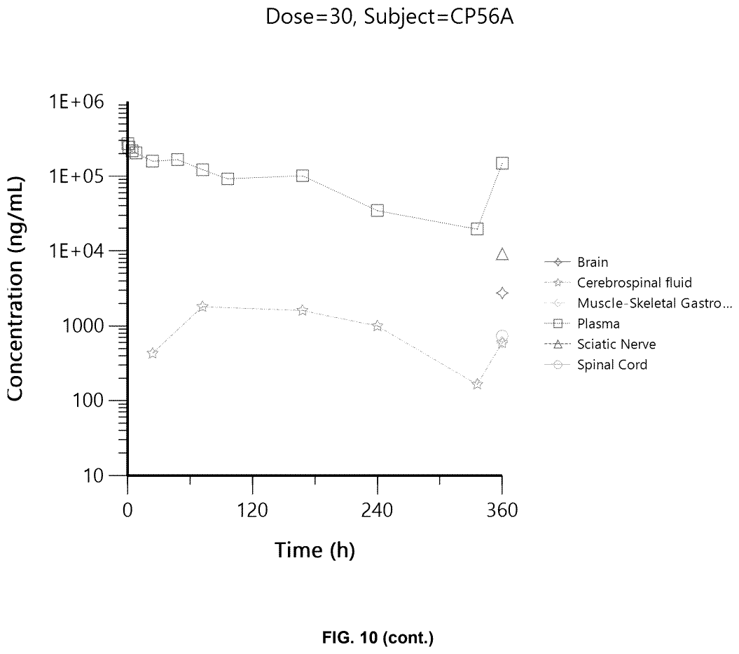

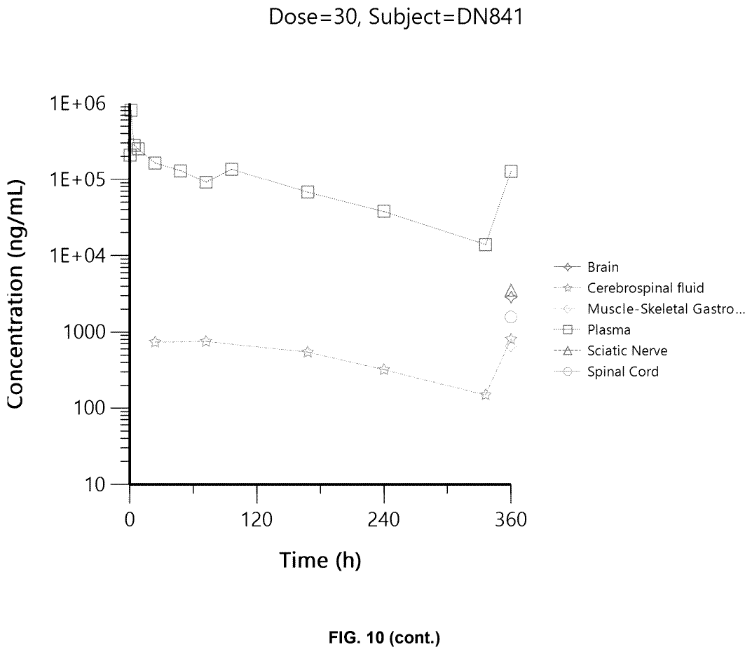

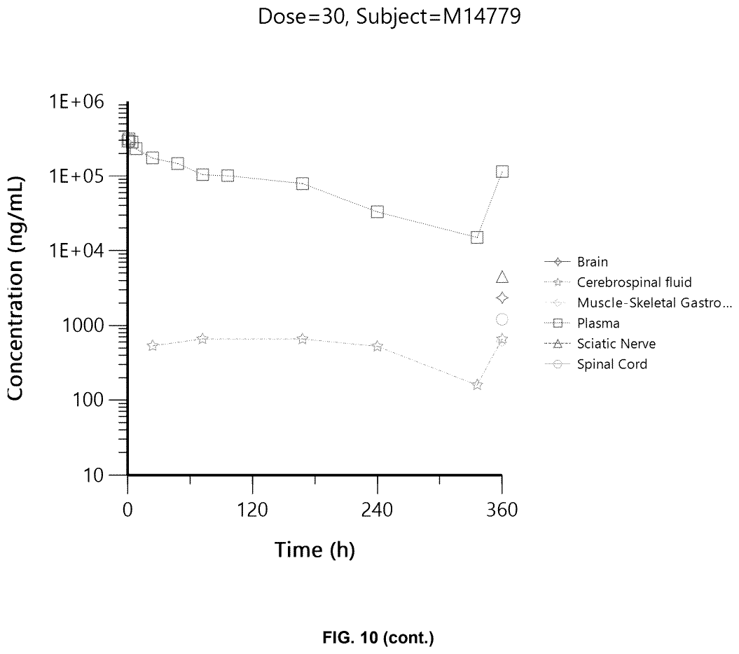

The bispecific anti-aC1s (L4)/anti-TfR (531v25.v6) IgG4 antibody was administered in monkeys at two dosages: 10 mg/kg and 30 mg/kg. Tissue was harvested at several timepoints following antibody administration. Plasma and cerebrospinal fluid exposure were measured after both the first and second administration of the bispecific antibody. Terminal tissue exposure (i.e., brain, sciatic nerve, spinal cord, and gastro muscle) were measured at 360 hours, about 24 hours following the second dose of antibody (FIG. 10, Table 10). The concentration of antibody in the various tissues and the tissue to plasma ratio of each tissue were measured and compared to a reference antibody (Shah and Betts, MAbs (2013) 5(2): 297-305). The lower limit of quantification (LLOQ) was 39.1 ng/mL for CSF, brain, sciatic nerve, spinal cord, and gastro muscle. The LLOQ was 156 ng/mL for plasma.

TABLE 10
Antibody Concentration and Tissue to Plasma Ratio

| | | Tissue to | |
|---|---|---|---|
| | Mean Conc. | Plasma Ratio | Tissue to Plasma |
| | (ng/mL or ng/g), | for Bispecific | Ratio for Reference |
| T = 360 hrs | n = 3 NHPs | Antibody | mAb |
| Plasma | 131000 ± 17691 | | |
| CSF | 688 ± 109 | 0.005 | |
| Brain | 2653 ± 268 | 0.020 | 0.003 |
| Sciatic | 5737 ± 3012 | 0.043 | NA |
| Nerve | | | |
| Spinal | 1175 ± 424 | 0.009 | NA |
| Cord | | | |
| Gastro | 628 ± 31 | 0.004 | 0.04 |
| Muscle | | | |

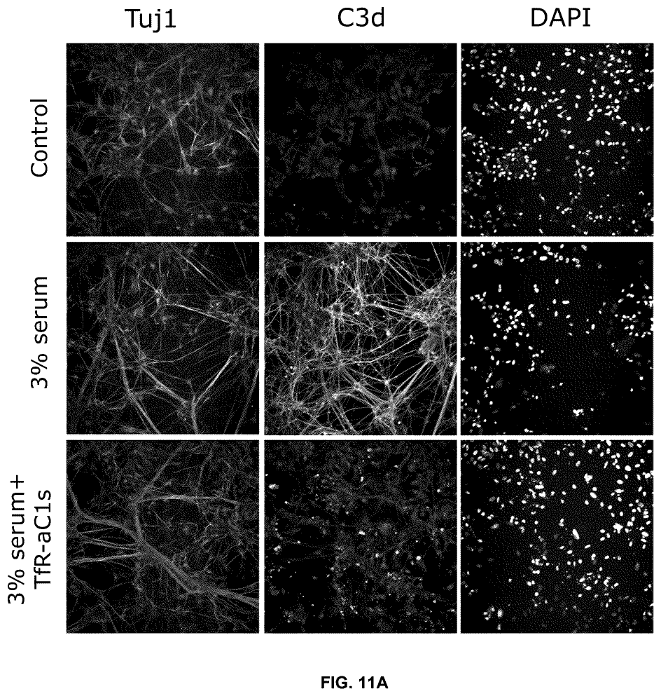

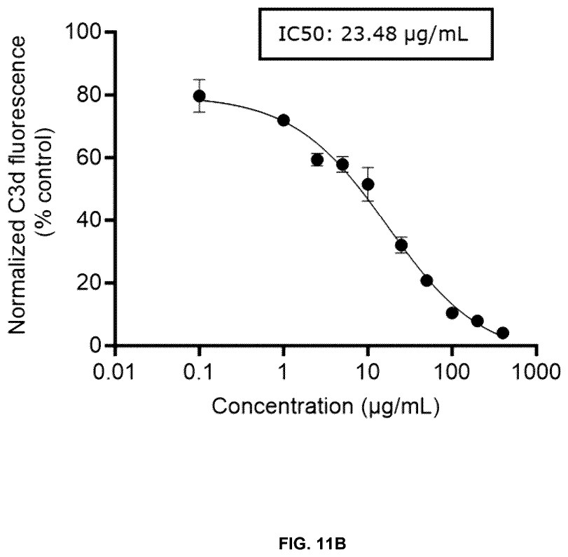

To further assess the potency of the bispecific anti-aC1s/anti-TfR antibody in a brain relevant human system, induced pluripotent stem cell brain tri-cultures (neurons, microglia, and astrocytes) were treated with either 3% serum or 3% serum and the bispecific antibody. The cells were imaged (FIG. 11A) and the C3d fluorescence measured at various concentrations of the bispecific antibody (FIG. 11B). The bispecific anti-aC1s (L4)/anti-TfR (531v25.v6) IgG4 antibody showed a dose-dependent inhibition of C3d deposition on the neurons in the tri-culture following complement activation (IC₅₀: 23.48 μg/mL).

### Sequences

Amino acid sequences provided in the present disclosure are listed below (SEQ: SEQ ID NO).

| | | |
|---|---|---|
| SEQ | Description | Sequence |

“088” aC1s-binding protein sequences

| | | |
|---|---|---|
| 1 | H-CDR1 | GFNIKDDY |
| | aC1s-D32E | |
| | aC1s-D32Y | |
| | aC1s-h088 | |
| | aC1s-088 | |
| | H5 | |
| | (IMGT) | |
| 2 | H-CDR2 | IDPADGHT |
| | aC1s-D32E | |
| | aC1s-D32Y | |
| | aC1s-h088 | |
| | aC1s-088 | |
| | H5 | |
| | (IMGT) | |
| 3 | H-CDR3 | ARYGYGREVF DY |
| | aC1s-D32E | |
| | aC1s-D32Y | |
| | aC1s-h088 | |
| | aC1s-088 | |
| | H5 | |
| | (IMGT) | |
| 4 | L-CDR1 | QSVDYEGDSY |
| | aC1s-D32E | |
| | L2 | |
| | (IMGT) | |
| 5 | L-CDR1 | QSVDYYGDSY |
| | aC1s-D32Y | |
| | (IMGT) | |
| 6 | L-CDR1 | QSVDYDGDSY |
| | aC1s-h088 | |
| | aC1s-088 | |
| | (IMGT) | |
| 7 | L-CDR2 | DAS |
| | aC1s-D32E | |
| | aC1s-D32Y | |
| | aC1s-h088 | |
| | aC1s-088 | |
| | L2 | |
| | (IMGT) | |
| 8 | L-CDR3 | QQSNEDPWT |
| | aC1s-D32E | |
| | aC1s-D32Y | |
| | aC1s-h088 | |
| | aC1s-088 | |
| | L2 | |
| | (IMGT/Kabat/Chothia) | |
| 9 | VH* (“HC”) | QVQLVQSGAE VKKPGASVKL SCTASGFNIK DDYIHWVKEA |
| | aC1s-D32E | PGQGLEWIGR IDPADGHTKY APKFQVKVTI TADTSTSTAY |
| | aC1s-D32Y | LELSSLRSED TAVYYCARYG YGREVFDYWG QGTTVTVSS |
| | aC1s-h088 | |
| | aC1s-088 | |
| 10 | VH | QVQLVQSGAE VKKPGASVKL SCTASGFNIK DDYIHWVKQA |
| | aC1s-D32E | PGQGLEWIGR IDPADGHTKY APKFQVKVTI TADTSTSTAY |
| | aC1s-D32Y | LELSSLRSED TAVYYCARYG YGREVFDYWG QGTTVTVSS |
| | aC1s-h088 | |
| | aC1s-088 | |
| 11 | VH* | QVQLVQSGAE VKKPGASVKL SCTASGFNIK DDYIHWVKEA |
| | H5 | PGQGLEWIGR IDPADGHTKY APKFQVKVTI TAATSTSTAY |
| | | LQLSSLRSED TAVYYCARYG YGREVFDYWG QGTTVTVSS |
| 12 | VH | QVQLVQSGAE VKKPGASVKL SCTASGFNIK DDYIHWVKQA |
| | H5 | PGQGLEWIGR IDPADGHTKY APKFQVKVTI TAATSTSTAY |
| | | LQLSSLRSED TAVYYCARYG YGREVFDYWG QGTTVTVSS |
| 13 | VL* | DIVMTQSPDS LAVSLGERAT ISCKSSQSVD YEGDSYLNWY |
| | aC1s-D32E | QKKPGQPPKL LIYDASNLES GVPDRFSGSG SGTDFTLTIS |
| | | SLQAEDVAVY YCQQSNEDPW TFGQGTKVEI K |
| 14 | VL | DIVMTQSPDS LAVSLGERAT ISCKSSQSVD YEGDSYLNWY |
| | aC1s-D32E | QQKPGQPPKL LIYDASNLES GVPDRFSGSG SGTDFTLTIS |
| | | SLQAEDVAVY YCQQSNEDPW TFGQGTKVEI K |
| 15 | VL* | DIVMTQSPDS LAVSLGERAT ISCKSSQSVD YYGDSYLNWY |
| | aC1s-D32Y | QKKPGQPPKL LIYDASNLES GVPDRFSGSG SGTDFTLTIS |
| | | SLQAEDVAVY YCQQSNEDPW TFGQGTKVEI K |
| 16 | VL | DIVMTQSPDS LAVSLGERAT ISCKSSQSVD YYGDSYLNWY |
| | aC1s-D32Y | QQKPGQPPKL LIYDASNLES GVPDRFSGSG SGTDFTLTIS |
| | | SLQAEDVAVY YCQQSNEDPW TFGQGTKVEI K |
| 17 | VL* | DIVMTQSPDS LAVSLGERAT ISCKSSQSVD YEGDSYLNWY |
| | L2 | QKKPGQPPKL LIYDASNLES GVPDRFSGSG SGTAFTLTIS |
| | | SLQAEDVAVY YCQQSNEDPW TFGQGTKVEI K |
| 18 | VL | DIVMTQSPDS LAVSLGERAT ISCKSSQSVD YEGDSYLNWY |
| | L2 | QQKPGQPPKL LIYDASNLES GVPDRFSGSG SGTAFTLTIS |
| | | SLQAEDVAVY YCQQSNEDPW TFGQGTKVEI K |
| 19 | VL* | DIVMTQSPDS LAVSLGERAT ISCKSSQSVD YDGDSYLNWY |
| | aC1s-h088 | QKKPGQPPKL LIYDASNLES GVPDRFSGSG SGTDFTLTIS |
| | | SLQAEDVAVY YCQQSNEDPW TFGQGTKVEI K |
| 20 | VL* | DIVLTQSPDS LAVSLGERAT ISCKASQSVD YDGDSYMNWY |
| | aC1s-088 | QKKPGQPPKI LIYDASNLES GIPARFSGSG SGTDFTLTIS |
| | | SLEPEDFAIY YCQQSNEDPW TFGGGTKVEI K |
| 21 | VL | DIVLTQSPDS LAVSLGERAT ISCKASQSVD YDGDSYMNWY |
| | aC1s-088 | QQKPGQPPKI LIYDASNLES GIPARFSGSG SGTDFTLTIS |
| | | SLEPEDFAIY YCQQSNEDPW TFGGGTKVEI K |

TfR-binding protein sequences

| | | |
|---|---|---|
| 22 | HCDR1 | GYTFTRYY |
| | 531v25.v6 | |
| | 531v23 | |
| | 531v25 | |
| | (IMGT) | |
| 23 | HCDR2 | IDPSVSET |
| | 531v25.v6 | |
| | 531v23 | |
| | 531v25 | |
| | (IMGT) | |
| 24 | HCDR3 | SQIRLPYYYAMDS |
| | 531v25.v6 | |
| | 531v23 | |
| | 531v25 | |
| | (IMGT) | |
| 25 | LCDR1 | QDIESF |
| | 531v25.v6 | |
| | (IMGT) | |
| 26 | LCDR1 | QDISSF |
| | 531v23 | |
| | 531v25 | |
| | (IMGT) | |
| 27 | LCDR2 | YTS |
| | 531v25.v6 | |
| | 531v23 | |
| | 531v25 | |
| | (IMGT) | |
| 28 | LCDR3 | QQGNTLPRT |
| | 531v25.v6 | |
| | 531v23 | |
| | 531v25 | |
| | (IMGT) | |
| 29 | VH* | QVQLVQSGAE VKKPGASVKV SCKASGYTFT RYYLNWVRKA |
| | 531v25.v6 | PGQGLEWIGM IDPSVSETHY AQKFQGRATL TVDKSTSTAY |
| | 531v23 | MELSSLRSED TAVYYCSQIR LPYYYAMDSW GQGTTVTVSS |
| | 531v25 | |
| 30 | VH | QVQLVQSGAE VKKPGASVKV SCKASGYTFT RYYLNWVRQA |
| | 531v25.v6 | PGQGLEWIGM IDPSVSETHY AQKFQGRATL TVDKSTSTAY |
| | 531v23 | MELSSLRSED TAVYYCSQIR LPYYYAMDSW GQGTTVTVSS |
| | 531v25 | |
| 31 | VL* | DIQMTQSPSS LSASVGDRVT ITCRASQDIE SFLNWYQEKP |
| | 531v25.v6 | GKAPKLLIYY TSRLQSGVPS RFSGSGSGTD YTLTISSLQP |
| | | EDFATYYCQQ GNTLPRTFGG GTKVEIK |
| 32 | VL | DIQMTQSPSS LSASVGDRVT ITCRASQDIE SFLNWYQQKP |
| | 531v25.v6 | GKAPKLLIYY TSRLQSGVPS RFSGSGSGTD YTLTISSLQP |
| | | EDFATYYCQQ GNTLPRTFGG GTKVEIK |
| 33 | VL* | DIQMTQSPSS LSASVGDRVT ITCRASQDIS SFLNWYQEKP |
| | 531v23 | GKAPKLLIYY TSRLHSGVPS RFSGSGSGTD YTLTISSLQP |
| | | EDFATYYCQQ GNTLPRTFGG GTKVEIK |
| 34 | VL | DIQMTQSPSS LSASVGDRVT ITCRASQDIS SFLNWYQQKP |
| | 531v23 | GKAPKLLIYY TSRLHSGVPS RFSGSGSGTD YTLTISSLQP |
| | | EDFATYYCQQ GNTLPRTFGG GTKVEIK |
| 35 | VL* | DIQMTQSPSS LSASVGDRVT ITCRASQDIS SFLNWYQEKP |
| | 531v25 | GKAPKLLIYY TSRLQSGVPS RFSGSGSGTD YTLTISSLQP |
| | | EDFATYYCQQ GNTLPRTFGG GTKVEIK |
| 36 | VL | DIQMTQSPSS LSASVGDRVT ITCRASQDIS SFLNWYQQKP |
| | 531v25 | GKAPKLLIYY TSRLQSGVPS RFSGSGSGTD YTLTISSLQP |
| | | EDFATYYCQQ GNTLPRTFGG GTKVEIK |

IgG1 and IgG4 heavy chain constant regions

| | | |
|---|---|---|
| 37 | IgG1 (wild-type) | ASTKGPSVFP LAPSSKSTSG GTAALGCLVK DYFPEPVTVS |
| | | WNSGALTSGV HTFPAVLQSS GLYSLSSVVT VPSSSLGTQT |
| | | YICNVNHKPS NTKVDKKVEP KSCDKTHTCP PCPAPELLGG |
| | | PSVFLFPPKP KDTLMISRTP EVTCVVVDVS HEDPEVKFNW |
| | | YVDGVEVHNA KTKPREEQYN STYRVVSVLT VLHQDWLNGK |
| | | EYKCKVSNKA LPAPIEKTIS KAKGQPREPQ VYTLPPSRDE |
| | | LTKNQVSLTC LVKGFYPSDI AVEWESNGQP ENNYKTTPPV |
| | | LDSDGSFFLY SKLTVDKSRW QQGNVFSCSV MHEALHNHYT |
| | | QKSLSLSPGK |
| 38 | IgG1-LALA-GASS | ASTKGPSVFP LAPSSKSTSG GTAALGCLVK DYFPEPVTVS |
| | | WNSGALTSGV HTFPAVLQSS GLYSLSSVVT VPSSSLGTQT |
| | | YICNVNHKPS NTKVDKKVEP KSCDKTHTCP PCPAPEAAGG |
| | | PSVFLFPPKP KDTLMISRTP EVTCVVVDVS HEDPEVKFNW |
| | | YVDGVEVHNA KTKPREEQYN STYRVVSVLT VLHQDWLNGK |
| | | EYKCKVSNKG LASSIEKTIS KAKGQPREPQ VYTLPPSRDE |
| | | LTKNQVSLTC LVKGFYPSDI AVEWESNGQP ENNYKTTPPV |
| | | LDSDGSFFLY SKLTVDKSRW QQGNVFSCSV MHEALHNHYT |
| | | QKSLSLSPG |
| 39 | IgG1-LALA-GASS-LS | ASTKGPSVFP LAPSSKSTSG GTAALGCLVK DYFPEPVTVS |
| | | WNSGALTSGV HTFPAVLQSS GLYSLSSVVT VPSSSLGTQT |
| | | YICNVNHKPS NTKVDKKVEP KSCDKTHTCP PCPAPEAAGG |
| | | PSVFLFPPKP KDTLMISRTP EVTCVVVDVS HEDPEVKFNW |
| | | YVDGVEVHNA KTKPREEQYN STYRVVSVLT VLHQDWLNGK |
| | | EYKCKVSNKG LASSIEKTIS KAKGQPREPQ VYTLPPSRDE |
| | | LTKNQVSLTC LVKGFYPSDI AVEWESNGQP ENNYKTTPPV |
| | | LDSDGSFFLY SKLTVDKSRW QQGNVFSCSV LHEALHSHYT |
| | | QKSLSLSPG |
| 40 | IgG4 (wild-type) | ASTKGPSVFP LAPCSRSTSE STAALGCLVK DYFPEPVTVS |
| | | WNSGALTSGV HTFPAVLQSS GLYSLSSVVT VPSSSLGTKT |
| | | YTCNVDHKPS NTKVDKRVES KYGPPCPSCP APEFLGGPSV |
| | | FLFPPKPKDT LMISRTPEVT CVVVDVSQED PEVQFNWYVD |
| | | GVEVHNAKTK PREEQFNSTY RVVSVLTVLH QDWLNGKEYK |
| | | CKVSNKGLPS SIEKTISKAK GQPREPQVYT LPPSQEEMTK |
| | | NQVSLTCLVK GFYPSDIAVE WESNGQPENN YKTTPPVLDS |
| | | DGSFFLYSRL TVDKSRWQEG NVFSCSVMHE ALHNHYTQKS |
| | | LSLSLGK |
| 41 | IgG1-knob | ASTKGPSVFP LAPSSKSTSG GTAALGCLVK DYFPEPVTVS |
| | | WNSGALTSGV HTFPAVLQSS GLYSLSSVVT VPSSSLGTQT |
| | | YICNVNHKPS NTKVDKKVEP KSCDKTHTCP PCPAPEAAGG |
| | | PSVFLFPPKP KDTLMISRTP EVTCVVVDVS HEDPEVKFNW |
| | | YVDGVEVHNA KTKPREEQYN STYRVVSVLT VLHQDWLNGK |
| | | EYKCKVSNKG LASSIEKTIS KAKGQPREPQ VYTLPPCRDE |
| | | LTKNQVSLWC LVKGFYPSDI AVEWESNGQP ENNYKTTPPV |
| | | LDSDGSFFLY SKLTVDKSRW QQGNVFSCSV LHEALHSHYT |
| | | QKSLSLSPG |
| 42 | IgG1-hole | ASTKGPSVFP LAPSSKSTSG GTAALGCLVK DYFPEPVTVS |
| | | WNSGALTSGV HTFPAVLQSS GLYSLSSVVE VPSSSLGTQT |
| | | YICNVNHKPS NTKVDEKVEP DSCDKTHTCP PCPAPEAAGG |
| | | PSVFLFPPKP KDTLMISRTP EVTCVVVDVS HEDPEVKFNW |
| | | YVDGVEVHNA KTKPREEQYN STYRVVSVLT VLHQDWLNGK |
| | | EYKCKVSNKG LASSIEKTIS KAKGQPREPQ VCTLPPSRDE |
| | | LTKNQVSLSC AVKGFYPSDI AVEWESNGQP ENNYKTTPPV |
| | | LDSDGSFFLV SKLTVDKSRW QQGNVFSCSV LHEALHSRFT |
| | | QKSLSLSPG |
| 43 | IgG4-knob | ASTKGPSVFP LAPCSRSTSE STAALGCLVK DYFPEPVTVS |
| | | WNSGALTSGV HTFPAVLQSS GLYSLSSVVT VPSSSLGTKT |
| | | YTCNVDHKPS NTKVDKRVES KYGPPCPPCP APEFEGGPSV |
| | | FLFPPKPKDT LMISRTPEVT CVVVDVSQED PEVQFNWYVD |
| | | GVEVHNAKTK PREEQFNSTY RVVSVLTVLH QDWLNGKEYK |
| | | CKVSNKGLPS SIEKTISKAK GQPREPQVYT LPPCQEEMTK |
| | | NQVSLWCLVK GFYPSDIAVE WESNGQPENN YKTTPPVLDS |
| | | DGSFFLYSRL TVDKSRWQEG NVFSCSVLHE ALHSHYTQKS |
| | | LSLSLG |
| 44 | IgG4-hole | ASTKGPSVFP LAPCSRSTSE STAALGCLVK DYFPEPVTVS |
| | | WNSGALTSGV HTFPAVLQSS GLYSLSSVVE VPSSSLGTKT |
| | | YTCNVDHKPS NTKVDERVES DYGPPCPPCP APEFEGGPSV |
| | | FLFPPKPKDT LMISRTPEVT CVVVDVSQED PEVQFNWYVD |
| | | GVEVHNAKTK PREEQFNSTY RVVSVLTVLH QDWLNGKEYK |
| | | CKVSNKGLPS SIEKTISKAK GQPREPQVCT LPPSQEEMTK |
| | | NQVSLSCAVK GFYPSDIAVE WESNGQPENN YKTTPPVLDS |
| | | DGSFFLVSRL TVDKSRWQEG NVFSCSVLHE ALHSRFTQKS |
| | | LSLSLG |
| 45 | Ig LC kappa | RTVAAPSVFIFPPSDEQLKSGTASVVCLLNNFYPREAKVQWKVD |
| | | NALQSGNSQESVTEQDSKDSTYSLSSTLTLSKADYEKHKVYACE |
| | | VTHQGLSSPVTKSFNRGEC |
| 46 | Ig LC kappa (with | RTVAAPAVFIFPPSKKQLKSGTASVVCLLKNFYPREAKVQWKVD |
| | charge-pair mutations) | NALQSGNSQESVTEQDSKDSTYSLSSTLTLSKADYEKHKVYACE |
| | | VTHQGLSSPVTKSFNRGEC |

Bispecific anti-aC1s/anti-TfR binding proteins

| | | |
|---|---|---|
| 47 | HC (aC1s) | QVQLVQSGAEVKKPGASVKLSCTASGFNIKDDYIHWVKEAPGQG |
| | HC | LEWIGRIDPADGHTKYAPKFQVKVTITADTSTSTAYLELSSLRS |
| | | EDTAVYYCARYGYGREVFDYWGQGTTVTVSSASTKGPSVFPLAP |
| | | CSRSTSESTAALGCLVKDYFPEPVTVSWNSGALTSGVHTFPAVL |
| | | QSSGLYSLSSVVEVPSSSLGTKTYTCNVDHKPSNTKVDERVESD |
| | | YGPPCPPCPAPEFEGGPSVFLFPPKPKDTLMISRTPEVTCVVVD |
| | | VSQEDPEVQFNWYVDGVEVHNAKTKPREEQFNSTYRVVSVLTVL |
| | | HQDWLNGKEYKCKVSNKGLPSSIEKTISKAKGQPREPQVCTLPP |
| | | SQEEMTKNQVSLSCAVKGFYPSDIAVEWESNGQPENNYKTTPPV |
| | | LDSDGSFFLVSRLTVDKSRWQEGNVFSCSVLHEALHSRFTQKSL |
| | | SLSLG |
| 48 | HC (aC1s) | QVQLVQSGAEVKKPGASVKLSCTASGFNIKDDYIHWVKEAPGQG |
| | H5 | LEWIGRIDPADGHTKYAPKFQVKVTITAATSTSTAYLQLSSLRS |
| | | EDTAVYYCARYGYGREVFDYWGQGTTVTVSSASTKGPSVFPLAP |
| | | CSRSTSESTAALGCLVKDYFPEPVTVSWNSGALTSGVHTFPAVL |
| | | QSSGLYSLSSVVEVPSSSLGTKTYTCNVDHKPSNTKVDERVESD |
| | | YGPPCPPCPAPEFEGGPSVELFPPKPKDTLMISRTPEVTCVVVD |
| | | VSQEDPEVQFNWYVDGVEVHNAKTKPREEQFNSTYRVVSVLTVL |
| | | HQDWLNGKEYKCKVSNKGLPSSIEKTISKAKGQPREPQVCTLPP |
| | | SQEEMTKNQVSLSCAVKGFYPSDIAVEWESNGQPENNYKTTPPV |
| | | LDSDGSFFLVSRLTVDKSRWQEGNVFSCSVLHEALHSRFTQKSL |
| | | SLSLG |
| 49 | LC (aC1s) | DIVMTQSPDSLAVSLGERATISCKSSQSVDYEGDSYLNWYQKKP |
| | L4 | GQPPKLLIYDASNLESGVPDRFSGSGSGTDFTLTISSLQAEDVA |
| | | VYYCQQSNEDPWTFGQGTKVEIKRTVAAPAVFIFPPSKKQLKSG |
| | | TASVVCLLKNFYPREAKVQWKVDNALQSGNSQESVTEQDSKDST |
| | | YSLSSTLTLSKADYEKHKVYACEVTHQGLSSPVTKSFNRGEC |
| 50 | LC (aC1s) | DIVMTQSPDSLAVSLGERATISCKSSQSVDYEGDSYLNWYQKKP |
| | L2 | GQPPKLLIYDASNLESGVPDRFSGSGSGTAFTLTISSLQAEDVA |
| | | VYYCQQSNEDPWTFGQGTKVEIKRTVAAPAVFIFPPSKKQLKSG |
| | | TASVVCLLKNFYPREAKVQWKVDNALQSGNSQESVTEQDSKDST |
| | | YSLSSTLTLSKADYEKHKVYACEVTHQGLSSPVTKSFNRGEC |
| 51 | HC (TfR) | QVQLVQSGAEVKKPGASVKVSCKASGYTFTRYYLNWVRKAPGQG |
| | 531v25.v6 | LEWIGMIDPSVSETHYAQKFQGRATLTVDKSTSTAYMELSSLRS |
| | | EDTAVYYCSQIRLPYYYAMDSWGQGTTVTVSSASTKGPSVFPLA |
| | | PCSRSTSESTAALGCLVKDYFPEPVTVSWNSGALTSGVHTFPAV |
| | | LQSSGLYSLSSVVTVPSSSLGTKTYTCNVDHKPSNTKVDKRVES |
| | | KYGPPCPPCPAPEFEGGPSVFLFPPKPKDTLMISRTPEVTCVVV |
| | | DVSQEDPEVQFNWYVDGVEVHNAKTKPREEQFNSTYRVVSVLTV |
| | | LHQDWLNGKEYKCKVSNKGLPSSIEKTISKAKGQPREPQVYTLP |
| | | PCQEEMTKNQVSLWCLVKGFYPSDIAVEWESNGQPENNYKTTPP |
| | | VLDSDGSFFLYSRLTVDKSRWQEGNVFSCSVLHEALHSHYTQKS |
| | | LSLSLG |
| 52 | LC (TfR) | DIQMTQSPSSLSASVGDRVTITCRASQDIESFLNWYQEKPGKAP |
| | 531v25.v6 | KLLIYYTSRLQSGVPSRFSGSGSGTDYTLTISSLQPEDFATYYC |
| | | QQGNTLPRTFGGGTKVEIKRTVAAPSVFIFPPSDEQLKSGTASV |
| | | VCLLNNFYPREAKVQWKVDNALQSGNSQESVTEQDSKDSTYSLS |
| | | STLTLSKADYEKHKVYACEVTHQGLSSPVTKSFNRGEC |

Target Sequences

| | | |
|---|---|---|
| 53 | human C1s | MWCIVLFSLLAWVYAEPTMYGEILSPNYPQAYPSEVEKSWDIEV |
| | | PEGYGIHLYFTHLDIELSENCAYDSVQIISGDTEEGRLCGQRSS |
| | | NNPHSPIVEEFQVPYNKLQVIFKSDFSNEERFTGFAAYYVATDI |
| | | NECTDFVDVPCSHFCNNFIGGYFCSCPPEYFLHDDMKNCGVNCS |
| | | GDVFTALIGEIASPNYPKPYPENSRCEYQIRLEKGFQVVVTLRR |
| | | EDFDVEAADSAGNCLDSLVFVAGDRQFGPYCGHGFPGPLNIETK |
| | | SNALDIIFQTDLTGQKKGWKLRYHGDPMPCPKEDTPNSVWEPAK |
| | | AKYVFRDVVQITCLDGFEVVEGRVGATSFYSTCQSNGKWSNSKL |
| | | KCQPVDCGIPESIENGKVEDPESTLFGSVIRYTCEEPYYYMENG |
| | | GGGEYHCAGNGSWVNEVLGPELPKCVPVCGVPREPFEEKQRIIG |
| | | GSDADIKNFPWQVFFDNPWAGGALINEYWVLTAAHVVEGNREPT |
| | | MYVGSTSVQTSRLAKSKMLTPEHVFIHPGWKLLEVPEGRTNFDN |
| | | DIALVRLKDPVKMGPTVSPICLPGTSSDYNLMDGDLGLISGWGR |
| | | TEKRDRAVRLKAARLPVAPLRKCKEVKVEKPTADAEAYVFTPNM |
| | | ICAGGEKGMDSCKGDSGGAFAVQDPNDKTKFYAAGLVSWGPQCG |
| | | TYGLYTRVKNYVDWIMKTMQENSTPRED |
| 54 | human TfR | MMDQARSAFSNLFGGEPLSYTRFSLARQVDGDNSHVEMKLAVDE |
| | | EENADNNTKANVTKPKRCSGSICYGTIAVIVFFLIGFMIGYLGY |
| | | CKGVEPKTECERLAGTESPVREEPGEDFPAARRLYWDDLKRKLS |
| | | EKLDSTDFTGTIKLLNENSYVPREAGSQKDENLALYVENQFREF |
| | | KLSKVWRDQHFVKIQVKDSAQNSVIIVDKNGRLVYLVENPGGYV |
| | | AYSKAATVTGKLVHANFGTKKDFEDLYTPVNGSIVIVRAGKITF |
| | | AEKVANAESLNAIGVLIYMDQTKFPIVNAELSFFGHAHLGTGDP |
| | | YTPGFPSFNHTQFPPSRSSGLPNIPVQTISRAAAEKLFGNMEGD |
| | | CPSDWKTDSTCRMVTSESKNVKLTVSNVLKEIKILNIFGVIKGF |
| | | VEPDHYVVVGAQRDAWGPGAAKSGVGTALLLKLAQMFSDMVLKD |
| | | GFQPSRSIIFASWSAGDFGSVGATEWLEGYLSSLHLKAFTYINL |
| | | DKAVLGTSNFKVSASPLLYTLIEKTMQNVKHPVTGQFLYQDSNW |
| | | ASKVEKLTLDNAAFPFLAYSGIPAVSFCFCEDTDYPYLGTTMDT |
| | | YKELIERIPELNKVARAAAEVAGQFVIKLTHDVELNLDYERYNS |
| | | QLLSFVRDLNQYRADIKEMGLSLQWLYSARGDFFRATSRLTTDF |
| | | GNAEKTDRFVMKKLNDRVMRVEYHFLSPYVSPKESPFRHVFWGS |
| | | GSHTLPALLENLKLRKQNNGAFNETLFRNQLALATWTIQGAANA |
| | | LSGDVWDIDNEF |

Alternative CDR definitions for aC1s-D32E

| | | |
|---|---|---|
| 55 | HCDR1 (Kabat) | DDYIH |
| 56 | HCDR2 (Kabat) | RIDPADGHTKYAPKFQV |
| 57 | HCDR3 | YGYGREVFDY |
| | (Kabat/Chothia) | |
| 58 | LCDR1 | KSSQSVDYEGDSYLN |
| | (Kabat/Chothia) | |
| 59 | LCDR2 | DASNLES |
| | (Kabat/Chothia) | |
| 60 | HCDR1 (Chothia) | GFNIKDD |
| 61 | HCDR2 (Chothia) | DPADGH |
| 62 | HCDR1 (Contact) | KDDYIH |
| 63 | HCDR2 (Contact) | WIGRIDPADGHTK |
| 64 | HCDR3 (Contact) | ARYGYGREVFD |
| 65 | LCDRI (Contact) | DYEGDSYLNWY |
| 66 | LCDR2 (Contact) | LLIYDASNLE |
| 67 | LCDR3 (Contact) | QQSNEDPW |

Alternative CDR definitions for 531v25.v6

| | | |
|---|---|---|
| 68 | HCDR1 (Kabat) | RYYLN |
| 69 | HCDR2 (Kabat) | MIDPSVSETHYAQKFQG |
| 70 | HCDR3 | IRLPYYYAMDS |
| | (Kabat/Chothia) | |
| 71 | LCDR1 | RASQDIESFLN |
| | (Kabat/Chothia) | |
| 72 | LCDR2 | YTSRLQS |
| | (Kabat/Chothia) | |
| 73 | HCDR1 (Chothia) | GYTFTRY |
| 74 | HCDR2 (Chothia) | DPSVSE |
| 75 | HCDR1 (Contact) | TRYYLN |
| 76 | HCDR2 (Contact) | WIGMIDPSVSETH |
| 77 | HCDR3 (Contact) | SQIRLPYYYAMD |
| 78 | LCDR1 (Contact) | ESFLNWY |
| 79 | LCDR2 (Contact) | LLIYYTSRLQ |
| 80 | LCDR3 (Contact) | QQGNTLPR |

“DAb” aC1s-binding protein sequences
DAb1

| | | |
|---|---|---|
| 81 | HCDR1 (IMGT) | GDTLTELS |
| 82 | HCDR2 (IMGT) | FDPEEGET |
| 83 | HCDR3 (IMGT) | VTEGLAGRPFDS |
| 84 | LCDR1 (IMGT) | QSISSW |
| 85 | LCDR2 (IMGT) | KAS |
| 86 | LCDR3 | QQYNSYSWT |
| | (IMGT/Kabat/Chothia) | |
| 87 | HCDR1 (Kabat) | ELSMH |
| 88 | HCDR2 (Kabat) | TEDPEEGETIYAQKFQG |
| 89 | HCDR3 | EGLAGRPFDS |
| | (Kabat/Chothia) | |
| 90 | LCDR1 | RASQSISSWLA |
| | (Kabat/Chothia) | |
| 91 | LCDR2 | KASSLES |
| | (Kabat/Chothia) | |
| 92 | HCDR1 (Chothia) | GDTLTEL |
| 93 | HCDR2 (Chothia) | DPEEGE |
| 94 | HCDR1 (Contact) | TELSMH |
| 95 | HCDR2 (Contact) | WMGTFDPEEGETI |
| 96 | HCDR3 (Contact) | VTEGLAGRPFD |
| 97 | LCDR1 (Contact) | SSWLAWY |
| 98 | LCDR2 (Contact) | LLIYKASSLE |
| 99 | LCDR3 (Contact) | QQYNSYSW |
| 100 | VH | QVQLVQSGAEVKKPGASVKVSCKVSGDTLTELSMHWVRQAPGKG |
| | | LEWMGTFDPEEGETIYAQKFQGRVTMTEDTSTDTAYMELSSLRS |
| | | EDTAVYYCVTEGLAGRPFDSWGQGTLVTVSS |
| 101 | VL | DIQMTQSPSTLSASVGDRVTITCRASQSISSWLAWYQQKPGKAP |
| | | KLLIYKASSLESGVPSRFSGSGSGTEFTLTISSLQPDDFATYYC |
| | | QQYNSYSWTFGQGTKVEIK |

DAb2

| | | |
|---|---|---|
| 102 | HCDR1 (IMGT) | GFTFSDYY |
| 103 | HCDR2 (IMGT) | ISRSGSTK |
| 104 | HCDR3 (IMGT) | ARDETDYALDY |
| 105 | LCDR1 (IMGT) | QDISNY |
| 106 | LCDR2 (IMGT) | DAS |
| 107 | LCDR3 | QQYEDLPLT |
| | (IMGT/Kabat/Chothia) | |
| 108 | HCDR1 (Kabat) | DYYMS |
| 109 | HCDR2 (Kabat) | YISRSGSTKYYADSVKG |
| 110 | HCDR3 | DETDYALDY |
| | (Kabat/Chothia) | |
| 111 | LCDR1 | QASQDISNYLN |
| | (Kabat/Chothia) | |
| 112 | LCDR2 | DASNLET |
| | (Kabat/Chothia) | |
| 113 | HCDR1 (Chothia) | GFTFSDY |
| 114 | HCDR2 (Chothia) | SRSGST |
| 115 | HCDR1 (Contact) | SDYYMS |
| 116 | HCDR2 (Contact) | WVSYISRSGSTKY |
| 117 | HCDR3 (Contact) | ARDETDYALD |
| 118 | LCDR1 (Contact) | SNYLNWY |
| 119 | LCDR2 (Contact) | LLIYDASNLE |
| 120 | LCDR3 (Contact) | QQYEDLPL |
| 121 | VH | QVQLVESGGGLVKPGGSLRLSCAASGFTFSDYYMSWIRQAPGKG |
| | | LEWVSYISRSGSTKYYADSVKGRFTISRDNAKNSLYLQMNSLRA |
| | | EDTAVYYCARDETDYALDYWGQGTLVTVSS |
| 122 | VL | DIQMTQSPSSLSASVGDRVTITCQASQDISNYLNWYQQKPGKAP |
| | | KLLIYDASNLETGVPSRFSGSGSGTDFTFTISSLQPEDIATYYC |
| | | QQYEDLPLTFGGGTKVEIK |

DAb3

| | | |
|---|---|---|
| 123 | HCDR1 (IMGT) | GFTFDDYG |
| 124 | HCDR2 (IMGT) | INWEGGST |
| 125 | HCDR3 (IMGT) | ARDEQLGGNYYYYYYMDV |
| 126 | LCDR1 (IMGT) | QGIRND |
| 127 | LCDR2 (IMGT) | TAS |
| 128 | LCDR3 | LQYNSYPLT |
| | (IMGT/Kabat/Chothia) | |
| 129 | HCDR1 (Kabat) | DYGMS |
| 130 | HCDR2 (Kabat) | GINWEGGSTGYADSVKG |
| 131 | HCDR3 | DEQLGGNYYYYYYMDV |
| | (Kabat/Chothia) | |
| 132 | LCDR1 | RASQGIRNDLG |
| | (Kabat/Chothia) | |
| 133 | LCDR2 | TASNLQS |
| | (Kabat/Chothia) | |
| 134 | HCDRI (Chothia) | GFTFDDY |
| 135 | HCDR2 (Chothia) | NWEGGS |
| 136 | HCDR1 (Contact) | DDYGMS |
| 137 | HCDR2 (Contact) | WVSGINWEGGSTG |
| 138 | HCDR3 (Contact) | ARDEQLGGNYYYYYYMD |
| 139 | LCDR1 (Contact) | RNDLGWY |
| 140 | LCDR2 (Contact) | RLIYTASNLQ |
| 141 | LCDR3 (Contact) | LQYNSYPL |
| 142 | VH | EVQLVESGGGVVRPGGSLRLSCAASGFTFDDYGMSWVRQAPGKG |
| | | LEWVSGINWEGGSTGYADSVKGRFTISRDNAKNSLYLQMNSLRA |
| | | EDTALYYCARDEQLGGNYYYYYYMDVWGKGTTVTVSS |
| 143 | VL | DIQMTQSPSSLSASVGDRVTITCRASQGIRNDLGWYQQKPGKAP |
| | | KRLIYTASNLQSGVPSRFSGSGSGTEFTLTISSLQPEDFATYYC |
| | | LQYNSYPLTFGGGTKVEIK |

DAb4

| | | |
|---|---|---|
| 144 | HCDR3 (IMGT) | ARDENYALDW |
| 145 | HCDR3 | DENYALDW |
| | (Kabat/Chothia) | |
| 146 | HCDR3 (Contact) | ARDENYALD |
| 147 | LCDR3 | QHYEDYPLT |
| | (IMGT/Kabat/Chothia) | |
| 148 | LCDR3 (Contact) | QHYEDYPL |
| 149 | VH | QVQLVESGGGLVKPGGSLRLSCAASGFTFSDYYMSWIRQAPGKG |
| | | LEWVSYISRSGSTKYYADSVKGRFTISRDNAKNSLYLQMNSLRA |
| | | EDTAVYYCARDENYALDWWGQGTLVTVSS |
| 150 | VL | DIQMTQSPSSLSASVGDRVTITCQASQDISNYLNWYQQKPGKAP |
| | | KLLIYDASNLETGVPSRFSGSGSGTDFTFTISSLQPEDIATYYC |
| | | QHYEDYPLTFGGGTKVEIK |

DAb5

| | | |
|---|---|---|
| 151 | VH | EVQLVESGGGLVKPGGSLRLSCAASGFTFSDYYMSWIRQAPGKG |
| | | LEWVSYISRSGSTKYYADSVKGRFTISRDNAKNSLYLQMNSLRA |
| | | EDTAVYYCARDENYALDWWGQGTLVTVSS |

SEQ: SEQ ID NO:
*: with charge mutation

1. An aC1s-binding protein comprising an anti-aC1s binding domain that comprises:
a) a heavy chain variable region (VH) comprising heavy chain complementarity-determining regions (HCDR) 1-3 set forth in SEQ ID NOs: 1, 2, and 3, respectively; and
a light chain variable region (VL) comprising light chain CDR (LCDR) 1-3 set forth in SEQ ID NOs: 4, 7 and 8, respectively; or
b) a VH comprising HCDR1-3 set forth in SEQ ID NOs: 1, 2, and 3, respectively; and a VL comprising LCDR1-3 set forth in SEQ ID NOs: 5, 7 and 8, respectively.

2. The aC1s-binding protein of claim 1, wherein
the VH comprises any one of SEQ ID NOs: 9-12; and
the VL comprises any one of SEQ ID NOs: 13-18.

3. The aC1s-binding protein of claim 1, wherein the VH and the VL comprise:
SEQ ID NOs: 9 and 13, respectively;
SEQ ID NOs: 9 and 15, respectively;
SEQ ID NOs: 9 and 17, respectively;
SEQ ID NOs: 10 and 14, respectively;
SEQ ID NOs: 10 and 16, respectively;
SEQ ID NOs: 10 and 18, respectively;
SEQ ID NOs: 11 and 13, respectively;
SEQ ID NOs: 11 and 15, respectively;
SEQ ID NOs: 11 and 17, respectively;
SEQ ID NOs: 12 and 14, respectively;
SEQ ID NOs: 12 and 16, respectively; or
SEQ ID NOs: 12 and 18, respectively.

4-8. (canceled)

9. The aC1s-binding protein of claim 1, wherein the aC1s-binding protein comprises an Fc region with one or both chains modified to bind a CNS target.

10-11. (canceled)

12. A TfR-binding protein comprising an anti-TfR binding domain that comprises:
a VH comprising HCDR1-3 set forth in SEQ ID NOs: 22, 23, and 24, respectively; and
a VL comprising LCDR1-3 set forth in SEQ ID NOs: 25, 27, and 28, respectively.

13. The TfR-binding protein of claim 12, wherein
the VH comprises SEQ ID NO: 29 or 30; and
the VL comprises any one of SEQ ID NOs: 31-34.

14. The TfR-binding protein of claim 12, wherein the VH and the VL comprise:
SEQ ID NOs: 29 and 31, respectively;
SEQ ID NOs: 29 and 33, respectively;
SEQ ID NOs: 30 and 32, respectively; or
SEQ ID NOs: 30 and 34, respectively.

15-18. (canceled)

19. The aC1s-binding protein of claim 1, wherein the aC1s-binding protein is an antibody of human isotype subclass IgG1, IgG2, IgG3, or IgG4.

20. The aC1s-binding protein of claim 19, wherein the antibody comprises
a) a human IgG1 or IgG4 constant region;
b) a human kappa light chain constant region; or
c) both a) and b).

21. The aC1s-binding protein of claim 19, comprising a human IgG4 constant region that comprises mutations selected from
i) S228P,
ii) L235E,
iii) M428L and N434S,
iv) H435R and Y436F, or
v) any combination of i)-iv),
wherein the mutation positions are according to Eu numbering.

22. (canceled)

23. The aC1s-binding protein of claim 19, comprising a human IgG4 heavy chain constant region that comprises SEQ ID NO: 40, optionally without the C-terminal lysine.

24. The aC1s-binding protein of claim 19, comprising a human IgG1 constant region that comprises mutations selected from
i) L234A and L235A,
ii) A237G, P329A, A330S, and P331S,
iii) M428L and N434S,
iv) H435R and Y436F, and
v) any combination of i)-iv),
wherein the mutation positions are according to Eu numbering.

25. (canceled)

26. The aC1s-binding protein of claim 19, comprising a human IgG1 heavy chain constant region that comprises any one of SEQ ID NOs: 37-39, optionally without the C-terminal lysine if present.

27. A bispecific binding protein comprising
a) an anti-aC1s binding domain, and
b) a binding domain specific for a CNS target.

28. (canceled)

29. The bispecific binding protein of claim 27, wherein the anti-aC1s binding domain comprises HCDR1-3 and LCDR1-3 set forth in
SEQ ID NOs: 1, 2, 3, 4, 7, and 8, respectively;
SEQ ID NOs: 1, 2, 3, 5, 7, and 8, respectively;
SEQ ID NOs: 1, 2, 3, 6, 7, and 8, respectively;
SEQ ID NOs: 81, 82, 83, 84, 85, and 86, respectively;
SEQ ID NOs: 102, 103, 104, 105, 106, and 107, respectively;
SEQ ID NOs: 123, 124, 125, 126, 127, and 128, respectively;
SEQ ID NOs: 102, 103, 144, 105, 106, and 147, respectively; or
SEQ ID NOs: 102, 103, 144, 105, 106, and 148, respectively.

30. The bispecific binding protein of claim 29, wherein the anti-aC1s binding domain comprises VH and VL that are at least 90% identical to
SEQ ID NOs: 9 and 13, respectively;
SEQ ID NOs: 9 and 15, respectively;
SEQ ID NOs: 9 and 17, respectively;
SEQ ID NOs: 9 and 19, respectively;
SEQ ID NOs: 9 and 20, respectively;
SEQ ID NOs: 10 and 14, respectively;
SEQ ID NOs: 10 and 16, respectively;
SEQ ID NOs: 10 and 18, respectively;
SEQ ID NOs: 10 and 21, respectively;
SEQ ID NOs: 11 and 13, respectively;
SEQ ID NOs: 11 and 15, respectively;
SEQ ID NOs: 11 and 17, respectively;
SEQ ID NOs: 11 and 19, respectively;
SEQ ID NOs: 11 and 20, respectively;
SEQ ID NOs: 12 and 14, respectively;
SEQ ID NOs: 12 and 16, respectively;
SEQ ID NOs: 12 and 18, respectively;
SEQ ID NOs: 12 and 21, respectively;
SEQ ID NOs: 100 and 101, respectively;
SEQ ID NOs: 121 and 122, respectively;
SEQ ID NOs: 142 and 143, respectively;
SEQ ID NOs: 149 and 150, respectively; or
SEQ ID NOs: 151 and 150, respectively.

31. The bispecific binding protein of claim 27, wherein the aC1s-binding domain comprises VH and VL set forth in
SEQ ID NOs: 9 and 13, respectively;
SEQ ID NOs: 9 and 15, respectively;
SEQ ID NOs: 9 and 17, respectively;
SEQ ID NOs: 9 and 19, respectively;
SEQ ID NOs: 9 and 20, respectively;
SEQ ID NOs: 10 and 14, respectively;
SEQ ID NOs: 10 and 16, respectively;
SEQ ID NOs: 10 and 18, respectively;
SEQ ID NOs: 10 and 21, respectively;
SEQ ID NOs: 11 and 13, respectively;
SEQ ID NOs: 11 and 15, respectively;
SEQ ID NOs: 11 and 17, respectively;
SEQ ID NOs: 11 and 19, respectively;
SEQ ID NOs: 11 and 20, respectively;
SEQ ID NOs: 12 and 14, respectively;
SEQ ID NOs: 12 and 16, respectively;
SEQ ID NOs: 12 and 18, respectively;
SEQ ID NOs: 12 and 21, respectively;
SEQ ID NOs: 100 and 101, respectively;
SEQ ID NOs: 121 and 122, respectively;
SEQ ID NOs: 142 and 143, respectively;
SEQ ID NOs: 149 and 150, respectively; or
SEQ ID NOs: 151 and 150, respectively.

32. The bispecific binding protein of claim 27, wherein the CNS target of the binding domain of b) is an endothelial cell receptor (ECR) of the blood brain barrier, optionally wherein the ECR is a transferrin receptor, insulin receptor, insulin-like growth factor receptor, low-density lipoprotein receptor, or folate receptor.

33. (canceled)

34. The bispecific binding protein of claim 32, wherein the ECR is transferrin receptor 1 (TfR), and the binding domain of b) is an anti-TfR binding domain.

35-36. (canceled)

37. The bispecific binding protein of claim 34, wherein the anti-TfR binding domain comprises HCDR1-3 and LCDR1-3 set forth in
SEQ ID NOs: 22, 23, 24, 25, 27, and 28, respectively; or
SEQ ID NOs: 22, 23, 24, 26, 27, and 28, respectively.

38. The bispecific binding protein of claim 37, wherein the anti-TfR binding domain comprises VH and VL at least 90% identical to
SEQ ID NOs: 29 and 31, respectively;
SEQ ID NOs: 29 and 33, respectively;
SEQ ID NOs: 29 and 35, respectively;
SEQ ID NOs: 30 and 32, respectively;
SEQ ID NOs: 30 and 34, respectively; or
SEQ ID NOs: 30 and 36, respectively.

39. The bispecific binding protein of claim 34, wherein the anti-TfR binding domain comprises VH and VL set forth in
SEQ ID NOs: 29 and 31, respectively;
SEQ ID NOs: 29 and 33, respectively;
SEQ ID NOs: 29 and 35, respectively;
SEQ ID NOs: 30 and 32, respectively;
SEQ ID NOs: 30 and 34, respectively; or
SEQ ID NOs: 30 and 36, respectively.

40. A bispecific binding protein that binds to aC1s and TfR, comprising
an anti-aC1s binding domain comprising HCDR1-3 and LCDR1-3 set forth in SEQ ID NOs: 1, 2, 3, 4, 7, and 8, respectively, and an anti-TfR binding domain comprising HCDR1-3 and LCDR1-3 set forth in SEQ ID NOs: 22, 23, 24, 25, 27, and 28, respectively;
an anti-aC1s binding domain comprising HCDR1-3 and LCDR1-3 set forth in SEQ ID NOs: 1, 2, 3, 4, 7, and 8, respectively, and an anti-TfR binding domain comprising HCDR1-3 and LCDR1-3 set forth in SEQ ID NOs: 22, 23, 24, 26, 27, and 28, respectively;
an anti-aC1s binding domain comprising HCDR1-3 and LCDR1-3 set forth in SEQ ID NOs: 1, 2, 3, 5, 7, and 8, respectively, and an anti-TfR binding domain comprising HCDR1-3 and LCDR1-3 set forth in SEQ ID NOs: 22, 23, 24, 25, 27, and 28, respectively;
an anti-aC1s binding domain comprising HCDR1-3 and LCDR1-3 set forth in SEQ ID NOs: 1, 2, 3, 5, 7, and 8, respectively, and an anti-TfR binding domain comprising HCDR1-3 and LCDR1-3 set forth in SEQ ID NOs: 22, 23, 24, 26, 27, and 28, respectively;
an anti-aC1s binding domain comprising HCDR1-3 and LCDR1-3 set forth in SEQ ID NOs: 1, 2, 3, 6, 7, and 8, respectively, and an anti-TfR binding domain comprising HCDR1-3 and LCDR1-3 set forth in SEQ ID NOs: 22, 23, 24, 25, 27, and 28, respectively;
an anti-aC1s binding domain comprising HCDR1-3 and LCDR1-3 set forth in SEQ ID NOs: 1, 2, 3, 6, 7, and 8, respectively, and an anti-TfR binding domain comprising HCDR1-3 and LCDR1-3 set forth in SEQ ID NOs: 22, 23, 24, 26, 27, and 28, respectively;
an anti-aC1s binding domain comprising HCDR1-3 and LCDR1-3 set forth in SEQ ID NOs: 81, 82, 83, 84, 85, and 86, respectively, and an anti-TfR binding domain comprising HCDR1-3 and LCDR1-3 set forth in SEQ ID NOs: 22, 23, 24, 25, 27, and 28, respectively;
an anti-aC1s binding domain comprising HCDR1-3 and LCDR1-3 set forth in SEQ ID NOs: 81, 82, 83, 84, 85, and 86, respectively, and an anti-TfR binding domain comprising HCDR1-3 and LCDR1-3 set forth in SEQ ID NOs: 22, 23, 24, 26, 27, and 28, respectively;
an anti-aC1s binding domain comprising HCDR1-3 and LCDR1-3 set forth in SEQ ID NOs: 102, 103, 104, 105, 106, and 107, respectively, and an anti-TfR binding domain comprising HCDR1-3 and LCDR1-3 set forth in SEQ ID NOs: 22, 23, 24, 25, 27, and 28, respectively;
an anti-aC1s binding domain comprising HCDR1-3 and LCDR1-3 set forth in SEQ ID NOs: 102, 103, 104, 105, 106, and 107, respectively, and an anti-TfR binding domain comprising HCDR1-3 and LCDR1-3 set forth in SEQ ID NOs: 22, 23, 24, 26, 27, and 28, respectively;
an anti-aC1s binding domain comprising HCDR1-3 and LCDR1-3 set forth in SEQ ID NOs: 123, 124, 125, 126, 127, and 128, respectively, and an anti-TfR binding domain comprising HCDR1-3 and LCDR1-3 set forth in SEQ ID NOs: 22, 23, 24, 25, 27, and 28, respectively;
an anti-aC1s binding domain comprising HCDR1-3 and LCDR1-3 set forth in SEQ ID NOs: 123, 124, 125, 126, 127, and 128, respectively, and an anti-TfR binding domain comprising HCDR1-3 and LCDR1-3 set forth in SEQ ID NOs: 22, 23, 24, 26, 27, and 28, respectively;
an anti-aC1s binding domain comprising HCDR1-3 and LCDR1-3 set forth in SEQ ID NOs: 102, 103, 144, 105, 106, and 147, respectively, and an anti-TfR binding domain comprising HCDR1-3 and LCDR1-3 set forth in SEQ ID NOs: 22, 23, 24, 25, 27, and 28, respectively;
an anti-aC1s binding domain comprising HCDR1-3 and LCDR1-3 set forth in SEQ ID NOs: 102, 103, 144, 105, 106, and 147, respectively, and an anti-TfR binding domain comprising HCDR1-3 and LCDR1-3 set forth in SEQ ID NOs: 22, 23, 24, 26, 27, and 28, respectively;
an anti-aC1s binding domain comprising HCDR1-3 and LCDR1-3 set forth in SEQ ID NOs: 102, 103, 144, 105, 106, and 148, respectively, and an anti-TfR binding domain comprising HCDR1-3 and LCDR1-3 set forth in SEQ ID NOs: 22, 23, 24, 25, 27, and 28, respectively; or
an anti-aC1s binding domain comprising HCDR1-3 and LCDR1-3 set forth in SEQ ID NOs: 102, 103, 144, 105, 106, and 148, respectively, and an anti-TfR binding domain comprising HCDR1-3 and LCDR1-3 set forth in SEQ ID NOs: 22, 23, 24, 26, 27, and 28, respectively.

41. The bispecific binding protein of claim 40, comprising
an anti-aC1s binding domain comprising VH and VL set forth in SEQ ID NOs: 9 and 13, respectively, and an anti-TfR binding domain comprising VH and VL set forth in SEQ ID NOs: 29 and 31, respectively;
an anti-aC1s binding domain comprising VH and VL set forth in SEQ ID NOs: 11 and 13, respectively, and an anti-TfR binding domain comprising VH and VL set forth in SEQ ID NOs: 29 and 31, respectively;
an anti-aC1s binding domain comprising VH and VL set forth in SEQ ID NOs: 9 and 17, respectively, and an anti-TfR binding domain comprising VH and VL set forth in SEQ ID NOs: 29 and 31, respectively;
an anti-aC1s binding domain comprising VH and VL set forth in SEQ ID NOs: 11 and 17, respectively, and an anti-TfR binding domain comprising VH and VL set forth in SEQ ID NOs: 29 and 31, respectively;
an anti-aC1s binding domain comprising VH and VL set forth in SEQ ID NOs: 100 and 101, respectively, and an anti-TfR binding domain comprising VH and VL set forth in SEQ ID NOs: 29 and 31, respectively;
an anti-aC1s binding domain comprising VH and VL set forth in SEQ ID NOs: 121 and 122, respectively, and an anti-TfR binding domain comprising VH and VL set forth in SEQ ID NOs: 29 and 31, respectively;
an anti-aC1s binding domain comprising VH and VL set forth in SEQ ID NOs: 142 and 143, respectively, and an anti-TfR binding domain comprising VH and VL set forth in SEQ ID NOs: 29 and 31, respectively;
an anti-aC1s binding domain comprising VH and VL set forth in SEQ ID NOs: 149 and 150, respectively, and an anti-TfR binding domain comprising VH and VL set forth in SEQ ID NOs: 29 and 31, respectively; or
an anti-aC1s binding domain comprising VH and VL set forth in SEQ ID NOs: 151 and 150, respectively, and an anti-TfR binding domain comprising VH and VL set forth in SEQ ID NOs: 29 and 31, respectively.

42-45. (canceled)

46. A bispecific binding protein comprising
a) a TfR-binding protein of claim 12 or an anti-TfR binding domain thereof, and
b) a binding domain specific for another, distinct target protein.

47. The bispecific binding protein of claim 46, wherein the distinct target protein is a protein of the complement system.

48. (canceled)

49. A bispecific binding protein comprising
a) an aC1s-binding protein of claim 1 or an anti-aC1s binding domain thereof, and
b) a binding domain specific for another, distinct target protein.

50. The bispecific binding protein of claim 49, wherein the distinct target protein is a CNS target protein, optionally wherein the CNS target protein is an endothelial cell receptor (ECR) of the blood brain barrier, optionally wherein the ECR is a transferrin receptor, insulin receptor, insulin-like growth factor receptor, low-density lipoprotein receptor, or folate receptor, optionally wherein the transferrin receptor is TfR.

51-65. (canceled)

66. The bispecific binding protein of claim 27, comprising
a first heavy chain that comprises SEQ ID NO: 47, a second heavy chain that comprises SEQ ID NO: 51, a first light chain that comprises SEQ ID NO: 49, and a second light chain that comprises SEQ ID NO: 52;
a first heavy chain that comprises SEQ ID NO: 47, a second heavy chain that comprises SEQ ID NO: 51, a first light chain that comprises SEQ ID NO: 50, and a second light chain that comprises SEQ ID NO: 52;
a first heavy chain that comprises SEQ ID NO: 48, a second heavy chain that comprises SEQ ID NO: 51, a first light chain that comprises SEQ ID NO: 49, and a second light chain that comprises SEQ ID NO: 52; or
a first heavy chain that comprises SEQ ID NO: 48, a second heavy chain that comprises SEQ ID NO: 51, a first light chain that comprises SEQ ID NO: 50, and a second light chain that comprises SEQ ID NO: 52.

67-69. (canceled)

70. A pharmaceutical composition comprising the aC1s-binding protein of claim 1 and a pharmaceutically acceptable excipient.

71. An isolated nucleic acid molecule(s) encoding the aC1s-binding protein of claim 1.

72. (canceled)

73. A host cell comprising the isolated nucleic acid molecule(s) of claim 71, optionally wherein the host cell is a mammalian cell.

74. A method of producing an aC1s-binding protein, a TfR-binding protein, or a bispecific binding protein, comprising:
culturing the host cell of claim 73 under conditions that allow expression of the binding protein, and
isolating the binding protein from the cell culture.

75. A method of treating a complement-mediated neurological disorder in a human subject in need thereof, comprising administering a therapeutically effective amount of the aC1s-binding protein of claim 1 to the subject.

76-77. (canceled)

78. The method of claim 75, wherein the complement-mediated neurological disorder is amyotrophic lateral sclerosis (ALS), Alzheimer's disease (AD), Huntington's disease (HD), an autoimmune peripheral neuropathy, a neurodegenerative eye disease, or dementia, optionally wherein the dementia is frontotemporal dementia (FTD).

79. (canceled)
